(19) United States
(12) Patent Application Publication
(10) Pub. No.: US 2024/0390902 A1
(43) Pub. Date: Nov. 28, 2024

(54) METHODS AND SYSTEMS FOR SORTING BIOLOGICAL PARTICLES

(71) Applicant: Newomics Inc., Berkeley, CA (US)

(72) Inventors: Daojing WANG, Moraga, CA (US); Yuchao CHEN, Rodeo, CA (US); Pan MAO, Milpitas, CA (US); Po Ying YEH, Pleasanton, CA (US)

(21) Appl. No.: 18/668,062

(22) Filed: May 17, 2024

Related U.S. Application Data
(63) Continuation-in-part of application No. 15/929,373, filed on Apr. 29, 2020, now abandoned.
Application No. 15/929,373 is a continuation of application No. PCT/US2018/059879, filed on Nov. 8, 2018.
(63) Continuation-in-part of application No. 17/946,892, filed on Sep. 16, 2022.
(60) Provisional application No. 62/583,949, filed on Nov. 9, 2017.
(60) Provisional application No. 63/280,950, filed on Nov. 18, 2021.

(51) Int. Cl. B01L 3/00

(52) U.S. Cl. CPC B01L 3/502746; B01L 2200/026; B01L 2400/086

(57) ABSTRACT

The present disclosure provides methods and systems for separating one or more target analytes from a fluid sample. The systems may comprise a microfluidic device. The microfluidic device may comprise a fluidic channel having an array of obstacles disposed therein. The array of obstacles may be oriented at an angle greater than 0° relative to a direction of a fluid flow in the fluidic channel. The array of obstacles may be configured to separate the target analytes from the fluid upon flow of the fluid through the fluidic channel. The methods of the present disclosure may comprise separating target analytes from a fluid using a microfluidic device comprising obstacles disposed in a fluidic channel of the device. The target analytes may be separated with a high efficiency, sensitivity and/or specificity. Also disclosed herein are devices, methods, and systems for separating one or more biological particles from a fluid sample. The devices may comprise a substrate with a fluidic channel disposed therein. The fluidic channel has disposed therein an array of obstacles with a vertical spacing. The vertical spacing may be configured to separate one or more particles from a fluid stream when the stream flows through the fluidic channel. The devices, methods, and systems may be able to separate various types of biological particles at a high efficiency, sensitivity, and/or specificity.

### CROSS-REFERENCE

This application is a continuation in part of U.S. patent application Ser. No. 15/929,373, filed Apr. 29, 2020, which is a continuation of International Patent Application No. PCT/US2018/059879, filed Nov. 8, 2018, which claims the benefit of U.S. Provisional Patent Application No. 62/583,949, filed Nov. 9, 2017, and U.S. patent application Ser. No. 17/946,892, filed Sep. 16, 2022, which claims the benefit of U.S. Provisional Patent Application No. 63/280,950, filed Nov. 18, 2021, all of which are incorporated herein by reference in their entirety.

### STATEMENT AS TO FEDERALLY SPONSORED RESEARCH

This invention was made with government support under grant numbers ES022360,ES023529, GM109682, AT008297, AG046025, and AI106100 and contract number HHSN261201300033C awarded by the National Institutes of Health. The government has certain rights in the invention.

### BACKGROUND

Separation and sorting of biological particles may be important for a variety of biomedical applications, including diagnostics, therapeutics, or fundamental cell biology. For example, understanding the causes underlying diseases may require separation of specific biological molecules or particles from complex samples, such as biofluids. Microfluidic-based methods and systems may be used for separating, capturing, detecting or analyzing biological molecules or particles.

### SUMMARY

Separation and sorting of biological particles may be important for a variety of biomedical applications, including diagnostics, therapeutics, or fundamental cell biology. Biological particles may include particles of biological origin. Non-limiting examples of biological particles may include cells or components thereof (e.g., nuclei), viruses, bacteria, proteins, carbohydrates, nucleic acid molecules (such as deoxyribonucleic acid (DNA), ribonucleic acid (RNA)), lipid, or combinations thereof. In some cases, cells may be senescent cells. In some cases, cells may be necrotic cells. In some cases, cell may comprise one or more of live cells, senescent cells, and necrotic cells.

Cellular senescence is a state of permanent cell cycle arrest due to genotoxic stresses and may be involved in organismal aging and tumorigenesis. Thus, senescent cell may be an important biomarker for aging as well as genotoxic stresses such as ionizing radiation. However, the small number of senescent cells in biofluids such as whole blood may limit their quick and sensitive detection.

Cellular senescence may play an important role in organismal aging and age-related diseases. However, it may be challenging to isolate low numbers of senescent cells from small volumes of biofluids for downstream analysis.

Recent animal studies have shown the potential of therapeutic targeting of senescent cells for anti-aging and age-related diseases. Because pathways up-or down-regulated in senescent cells, such as those involving p16, p21, and p53, may also function at various degrees in their healthy counterparts throughout the tissues and organs, conventional methods that target these pathways with small molecules and protein drugs may result in side effects in humans.

Different microfluidic techniques may be utilized for cell separation based on their physical properties (size, deformability, density, etc.), including filtration, deterministic lateral displacement (DLD), inertial flow, and acoustofluidics. Filtration may be used to process undiluted whole blood for rare cell separation and easily scaled up for a higher throughput. However, several challenges may exist. In dead-end flow filtration which may have the flow direction perpendicular to the filter surface, common issues may comprise the clogging and saturation of the filter, resulting in a lower separation efficiency, sample purity, and device robustness. A periodic reversed flow or fluidic oscillation may be adopted to address clogging. However, it may reduce the separation throughput and operation simplicity. Additionally, cell integrity may be decreased as they squeeze through the filtration pores, which may result in the changes of cell cytoskeleton. Crossflow filtration in microfluidics with a flow direction parallel to the filter surface may be used to decrease cell damage and clogging issue, and a shear force may be generated to bring the bigger particles to the downstream instead of entering the filtration pores. However, to ensure effective cell separation in a parallel-flow configuration, the crossflow filtration may require a much longer channel with a throughput lower than 1 milliliter/hour (mL/h). Despite the low throughput for microfluidic devices, a higher throughput (e.g. >1 mL/min) may be highly desired in order to process a large volume of complex samples such as whole blood samples. High throughput may be particularly challenging for a continuous flow due to difficulties in system integration and fluidic control for multiplexing on a microfluidic chip. Accordingly, recognized herein is a need for a platform that can achieve specificity, sensitivity, and throughput for isolation and removal of diverse “toxic” cells from biofluids.

The present disclosure provides effective isolation approaches for senescent-cell-based point-of-care diagnostics, such as radiation biodosimetry. The present disclosure provides methods and systems that can selectively remove senescent cells in a high-throughput manner.

The present disclosure provides methods and systems for separating, capturing, detecting and/or analyzing biological particles, molecules or components. The methods and systems may be used for radiation biodosimetry, radiological/nuclear medical countermeasures, anti-aging and anti-cancer therapies. The methods and systems may comprise the use of microfluidic-based platform. The microfluidic-based platform may comprise microfluidic devices. The microfluidic devices may comprise one or more microfluidic channels which may comprise one or more obstacles. The one or more obstacles may comprise an array of obstacles. The array of obstacles may be a three-dimensional (3D) array. The microfluidic devices may comprise an integrated microfluidic chip. The microfluidic devices may be monolithic. The microfluidic chip may be used for on-chip and online biological particle separation. The microfluidic devices may be used for single-cell analysis. The microfluidic devices may be used for biological particle separation in small volumes of fluid samples. In some cases, the microfluidic devices may be used for ultrahigh-throughput size-based isolation and removal of diverse cells in biofluids. The microfluidic devices of the present disclosure can be used for processing samples of various volumes and/or quantities. For example, the microfluidic devices can be used for processing both small-volume and large-volume samples.

Additionally or alternatively, the microfluidic-based platform may comprise multi-unit, large-dimension microfluidic chips. Such microfluidic chips may be used for ultrahigh-throughput parallel particle isolation and removal. The samples from which the particles are removed may have a large volume.

An aspect of the present disclosure provides a microfluidic device comprising: a fluidic channel; and an array of obstacles disposed in the fluidic channel, wherein the array of obstacles is oriented at an angle greater than 0° relative to a direction of a fluid flow in the fluidic channel; wherein the array of obstacles is configured to separate one or more target analytes from a fluid flowing through the fluidic channel.

In some embodiments, the angle is between about 1° and 85°. In some embodiments, the angle is between about 5° and 30°. In some embodiments, a distance between the array of obstacles and a side wall of the fluidic channel increases along the direction of the fluid flow. In some embodiments, at least one obstacle of the array of obstacles is adjacent to a side wall of the fluidic channel. In some embodiments, individual obstacles of the array of obstacles have a quadrilateral cross-section. In some embodiments, the quadrilateral cross-section is a parallelogram cross-section. In some embodiments, the array of obstacles is slanted in vertical or various angular directions. In some embodiments, an average spacing size between obstacles of the array is between about 100 nanometers and 100 micrometers (μm). In some embodiments, the average spacing size is between about 1 μm and 100 μm. In some embodiments, individual obstacles of the array have spaces configured to separate the one or more target analytes from the fluid. In some embodiments, the array of obstacles has a height less than or equal to a height of the fluidic channel. In some embodiments, the array of obstacles is configured to direct the one or more target analytes to flow at a direction different from the direction of the fluid flow. In some embodiments, the array of obstacles is configured to separate the one or more target analytes from the fluid based at least partially on a size of the one or more target analytes. In some embodiments, the size of the one or more target analytes is greater than or equal to a threshold value. In some embodiments, the one or more target analytes comprise biological particles. In some embodiments, the one or more target analytes comprise cells. In some embodiments, the cells comprise senescent cells. In some embodiments, the fluid comprises a biofluid. In some embodiments, the biofluid comprises whole blood. In some embodiments, the whole blood is undiluted or diluted. In some embodiments, the array of obstacles comprises microstructures. In some embodiments, the microstructures comprise micropillars. In some embodiments, the microstructures are three-dimensional (3D) microstructures. In some embodiments, obstacles of the array of obstacles have an average size between about 1 μm and about 100 μm. In some embodiments, at least a subset of the array of obstacles deforms when a flow rate of the fluid is greater than a threshold value. In some embodiments, obstacles of the array of obstacles are non-porous. In some embodiments, the microfluidic device further comprises one or more fluid inlets in fluidic communication with the fluidic channel. In some embodiments, the microfluidic device further comprises at least a first fluid outlet and a second fluid outlet in fluidic communication with the fluidic channel. In some embodiments, the first fluid outlet is configured to receive the one or more target analytes and the second fluid outlet is configured to receive the fluid absent the one or more target analytes. In some embodiments, the microfluidic device further comprises a fluidic component in fluidic communication with the fluidic channel. In some embodiments, the fluidic component is configured to receive and remove the one or more target analytes from the fluidic channel. In some embodiments, the fluidic component is a tubing. In some embodiments, the microfluidic device further comprises an additional fluidic channel in fluidic communication with the fluidic channel. In some embodiments, the additional fluidic channel is configured to receive and retain the one or more target analytes. In some embodiments, the microfluidic device further comprises an additional array of obstacles disposed therein. In some embodiments, the additional array of obstacles is configured to capture the one or more target analytes. In some embodiments, each of the additional array of obstacles has an opening. In some embodiments, the opening has a dimension greater than or equal to a size of the one or more target analytes. In some embodiments, the microfluidic device comprises a plurality of microfluidic channels each comprising a different array of obstacles. In some embodiments, each of the plurality of microfluidic channels is configured to separate a given type of target analytes from a fluid flowing therethrough. In some embodiments, the microfluidic device further comprises a body structure comprising a substrate. In some embodiments, the fluidic channel is disposed within the substrate. In some embodiments, the microfluidic device further comprises one or more fluidic pumps configured to transport fluids within the microfluidic device. In some embodiments, the one or more fluidic pumps comprise a plurality of valves.

Another aspect of the present disclosure provides a microfluidic device comprising: a fluidic channel; and an array of obstacles disposed in the fluidic channel; wherein the array of obstacles is configured to separate one or more particles from a fluid flowing through the fluidic channel with an efficiency of greater than about 70% at a flow rate of greater than or equal to about 250 milliliters/hour (mL/hr).

In some embodiments, the fluid is whole blood. In some embodiments, the one or more particles comprise senescent cells. In some embodiments, the one or more particles have an average size greater than or equal to about 25 micrometers. In some embodiments, the fluid has a volume more than or equal to about 30 milliliters. In some embodiments, the efficiency is greater than about 85%. In some embodiments, the flow rate is greater than or equal to about 300 ml/hr.

Another aspect of the present disclosure provides a microfluidic device comprising: a fluidic channel; and an array of obstacles disposed in the fluidic channel; wherein the array of obstacles is configured to separate one or more senescent cells from a fluid having a volume less than or equal to about 1 milliliter (mL) at an efficiency greater than about 70% upon flow of the fluid through the fluidic channel.

In some embodiments, the efficiency is greater than about 85%. In some embodiments, less than 25 mol % of non-senescent cells are separated from the fluid. In some embodiments, the fluid comprises biofluids. In some embodiments, the biofluids comprise whole blood or bone marrow. In some embodiments, the whole blood is undiluted. In some embodiments, the volume is less than or equal to about 50 microliters (μL). In some embodiments, the volume is less than or equal to about 25 μL. In some embodiments, the volume is less than or equal to about 5 μL. In some embodiments, the array of obstacles comprises microstructures. In some embodiments, the microstructures comprise micropillars. In some embodiments, the microstructures are three-dimensional (3D) microstructures. In some embodiments, obstacles of the array of obstacles are nonporous. In some embodiments, an average spacing size between obstacles of the array is between about 100 nanometers and 100 micrometers (μm). In some embodiments, the average spacing size is between about 1 μm and 100 μm. In some embodiments, the one or more senescent cells comprise senescent T cells, different kinds of white blood cells, microphages, lung, breast, colon, prostate, gastric, hepatic, ovarian, esophageal, or bronchial epithelial or stromal cells, senescent skin epithelial or stromal cells, senescent glial cells, senescent vascular endothelial or stromal cells, or combinations thereof.

Another aspect of the present disclosure provides a method comprising: (a) directing a fluid comprising one or more target analytes into a microfluidic device, the microfluidic device comprising: a fluidic channel; and an array of obstacles disposed in the fluidic channel, wherein the array of obstacles is oriented at an angle greater than 0° relative to a direction of a fluid flow in the fluidic channel; (b) directing the fluid to flow through the fluidic channel; and (c) separating at least a portion of the one or more target analytes from the fluid using the array of obstacles upon flow of the fluid through the fluidic channel.

In some embodiments, the angle is between about 1° and 85°. In some embodiments, the angle is between about 5° and 30°. In some embodiments, a distance between the array of obstacles and a side wall of the fluidic channel increases along the direction of the fluid flow. In some embodiments, at least one obstacle of the array of obstacles is adjacent to a side wall of the fluidic channel. In some embodiments, individual obstacles of the array of obstacles have a quadrilateral cross-section. In some embodiments, the quadrilateral cross-section is a parallelogram cross-section. In some embodiments, the one or more target analytes comprise biological particles. In some embodiments, the one or more target analytes comprise cells. In some embodiments, the cells comprise senescent cells. In some embodiments, the method further comprises directing an additional fluid into the microfluidic device. In some embodiments, the additional fluid is a sheath fluid. In some embodiments, the sheath fluid comprises a buffer. In some embodiments, the method further comprises capturing the at least the portion of the one or more target analytes. In some embodiments, the method further comprises subjecting the at least the portion of the one or more target analytes to further analyses. In some embodiments, the method further comprises removing the at least the portion of the one or more target analytes from the microfluidic device. In some embodiments, the at least the portion of the one or more target analytes is separated at a sensitivity of at least about 70%. In some embodiments, the at least the portion of the one or more target analytes is separated at a specificity of at least about 70%. In some embodiments, the method further comprises detecting the at least the portion of the one or more target analytes.

Another aspect of the present disclosure provides a method comprising: (a) directing a fluid comprising one or more particles into a microfluidic device, the microfluidic device comprising: a fluidic channel; and an array of obstacles disposed in the fluidic channel; (b) directing the fluid to flow through the fluidic channel; and (c) upon flow of the fluid through the fluidic channel, using the array of obstacles to separate the one or more particles from the fluid with an efficiency of greater than about 70% at a flow rate of greater than or equal to about 250 milliliters/hour (ml/hr).

In some embodiments, the fluid is whole blood. In some embodiments, the one or more particles comprise senescent cells. In some embodiments, the one or more particles have an average size greater than or equal to about 25 micrometers. In some embodiments, the fluid has a volume greater than or equal to about 30 milliliters. In some embodiments, the efficiency is greater than about 85%. In some embodiments, the flow rate is greater than or equal to about 300 ml/hr.

Another aspect of the present disclosure provides a method comprising: (a) directing a fluid having a volume less than or equal to about 1 milliliter (mL) into a microfluidic device, wherein the fluid comprise one or more senescent cells and wherein the microfluidic device comprises: a fluidic channel; and an array of obstacles disposed in the fluidic channel; (b) directing the fluid to flow through the fluidic channel; and (c) using the array of obstacles to separate at least a portion of the one or more senescent cells from the fluid at an efficiency greater than about 70% upon flow of the fluid through the fluidic channel.

In some embodiments, the efficiency is greater than about 85%. In some embodiments, less than 25 mol % of non-senescent cells are separated from the fluid. In some embodiments, the fluid comprises biofluids. In some embodiments, the biofluids comprise whole blood or bone marrow. In some embodiments, the whole blood is undiluted. In some embodiments, the volume is less than or equal to about 50 microliters (μL). In some embodiments, the volume is less than or equal to about 25 μL. In some embodiments, the volume is less than or equal to about 5 μL. In some embodiments, the method further comprises directing an additional fluid into the microfluidic device. In some embodiments, the additional fluid is a sheath fluid. In some embodiments, (c) is conducted based at least partially on a size of the one or more senescent cells. In some embodiments, the array of obstacles comprises microstructures. In some embodiments, the microstructures comprise micropillars. In some embodiments, the microstructures are three-dimensional (3D) microstructures. In some embodiments, obstacles of the array of obstacles are nonporous. In some embodiments, an average spacing size between obstacles of the array is between about 100 nanometers and 100 micrometers (μm). In some embodiments, the average spacing size is between about 1 μm and 100 μm. In some embodiments, (c) comprises using the array of obstacles to cause the at least the portion of the one or more senescent cells to flow at a direction that is different from a direction of the fluid in the fluidic channel. In some embodiments, the method further comprises capturing the at least the portion of the one or more senescent cells. In some embodiments, the method further comprises subjecting the at least the portion of the one or more senescent cells to further analyses. In some embodiments, the method further comprises removing the at least the portion of the one or more senescent cells from the microfluidic device. In some embodiments, the at least the portion of the one or more senescent cells is separated at a sensitivity of at least about 70%. In some embodiments, the at least the portion of the one or more senescent cells is separated at a specificity of at least about 70%. In some embodiments, the method further comprises detecting the at least the portion of the one or more senescent cells. In some embodiments, the detecting is at a single-cell resolution. In some embodiments, the one or more senescent cells comprise senescent T cells, different kinds of white blood cells, microphages, lung, breast, colon, prostate, gastric, hepatic, ovarian, esophageal, or bronchial epithelial or stromal cells, senescent skin epithelial or stromal cells, senescent glial cells, senescent vascular endothelial or stromal cells, or combinations thereof.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Live cells acquire different fates including apoptosis, necrosis, and senescence in response to stress and stimuli. Rapid and label-free enrichment of live cells from a mixture of cells adopting various cell fates remains a challenge. For example, purification of viable cells may serve as the first step for cancer drug screening and cell therapy. At least 85% cell viability for live cells may be required for reproducible cancer drug screenings. Current standard cell sorting techniques, such as flow cytometry and magnetic-activated cell sorting devices, may require labeling with single or multiple “tags” or “labels” to identify cells of interest. Further limitations in these cell sorting platforms for clinical sample processing can include (a) requirement of a large number of cells; (b) bulky instrumentation that occupies large bench footprints; (c) high operating pressures that could result in a loss of cell function or cell viability; and (d) increased risks of sample contamination in the lengthy process. Label-free cell sorting methods that utilize the physical characteristics of cells can overcome the aforementioned limitations. Depending on the existence of the external force fields, label-free cell sorting methods can be classified into two main categories: passive and active methods. Active cell sorting may be by mechanisms including acoustophoresis, dielectrophoresis, magnetophoresis, and centrifugation. On the other hand, passive cell sorting methods include, for example, filtration, geometries of microstructure, deterministic lateral displacement (DLD), initial microfluidics including spiral microchannel, contraction-expansion structure, and serpentine microchannel.

Passive cell sorting methods may not require the use of chemical reagents and may have other advantages including short sample preparation time, no external force field operation, relatively high processing throughput, and high cell viability. For example, microfluidics using the deterministic lateral displacement (DLD) mechanism may process at a high flow rate with high accuracy and offer the capacity for sorting cells of a similar size. However, DLD devices can require complex structures and long channels, which can lead to excessive fluid resistance and the requirement of high driving pressures. Furthermore, a single DLD device has difficulty in processing whole blood samples due to clogging and cell-cell collisions. Inertial microfluidics is another type of passive method. It can achieve relatively high throughput and may be easy to be integrated with other cell sorting methods. However, its cell sorting performance may be affected by cell size and flow velocity. In addition, cell separation solely based on the size difference may be insufficient for achieving high-accuracy separation owing to the heterogeneity of cells. Furthermore, the purity of target cells may be impaired when they are sorted from undiluted blood due to the cell-cell collisions. Filtration, on the other hand, has the advantages of a relatively simple structure amenable for mass production. However, for one-dimensional filters, clogging may occur while processing high-density cell samples. Crossflow filtration, another type of filtration, offers potential for sorting similar-sized cells. However, the cell attachment to micropillar or weir can still block the channel and the deformed target cells might pass through the pores or weirs, which can result in reduced sorting efficiency. Among reported passive cell sorting methods, it remains very challenging to enrich viable cells from a complex cellular mixture containing healthy, senescent, and necrotic cell populations of similar size through a single microfluidic chip.

Accordingly, the present disclosure describes methods, systems, and devices for complex cell separation which may be achieved by taking advantage of the increased cell size characteristic of senescence, as well as decreased cell size observed in the later stages of necrosis. Disclosed herein are devices, systems, and methods for size-based cell separation which may increase separation resolution and efficiency. Methods, systems, and devices as described herein may demonstrate reduced cell clogging when loading cells at a high concentration (>10⁴/mL) compared to conventional methods, systems, and devices. Devices as described herein may include a tunable vertical spacing (or “z-gap”) to allow small cells to flow through without experiencing the high pressure that might cause cell damage. Devices, systems, and methods as described herein may comprise a peristaltic pump to reduce the risk of contamination during cell sorting. Devices as described herein may operate at a flow rate of, e.g., 50 μL/min or more for label-free separation and enrichment of viable cells from a complex mixture of healthy, senescent, and necrotic mesenchymal stem cells. Methods and systems of the present disclosure may be able to separate live, senescent, and necrotic cells. Systems of the present disclosure may sort cells in a continuous flow format.

Non-limiting examples of features of devices of the present disclosure include a slanted and tunable 3D filter array in the vertical direction (z-gap) for rapid and continuous cell sieving. The shape of the 3D filter array may be optimized for target cells to prevent clogging during continuous separation. Such devices may demonstrate enrichment of live cells, as well as the removal of senescent and necrotic cells, achieving a high enrichment efficiency with high continuous flow. The devices may be used, for example, for cell-based drug screening for anti-cancer and anti-aging cell therapies.

An aspect of the present disclosure provides for a fluidic device comprising: a substrate; a fluidic channel disposed in or along the substrate, wherein the fluidic channel comprises: a top surface defining a ceiling of the fluidic channel opposing a surface of the substrate; a main channel comprising: an inlet; an outlet; a length defining an x-axis direction; a width defining a y-axis direction; a height defining a z-axis direction; a first side channel connected to the outlet of the main channel; a second side channel connected to the outlet of the main channel; and an array of first obstacles disposed in the main channel, extending from the ceiling and toward the surface of the substrate substantially along the z-axis, wherein each first obstacle comprises a first length along the z-axis, and wherein the first length is shorter than the height of the main channel, thereby separating the each first obstacle from the surface of the substrate by a first vertical spacing.

In some cases, the array of the first obstacles comprises at least a first line of the first obstacles and a second line of the first obstacles, wherein the first line of the first obstacles is separated from the second line of the first obstacles by at least a first distance along the y-axis. In some cases, the first distance along the y-axis is at least about 10 nanometers (nm), 20 nm, 40 nm, 50 nm, 60 nm, 70 nm, 80 nm, 90 nm, 100 nm, 200 nm, 300 nm, 500 nm, 1 micrometer (μm), 2 μm, 5 μm, 10 μm, 30 μm, 50 μm, or 100 μm. In some cases, the first line of the first obstacles is at an angle of θ₁ relative to the x-axis on a plane defined by the x-axis and the y-axis. In some cases, at least a subset of the first line of the first obstacles comprises a substantially similar parallelogram cross section on the plane, wherein the parallelogram comprises an acute angle of θ₂, and wherein θ₂ is larger than θ₁. In some cases, the angle θ₁ is from about 1° to about 85° relative to the direction of the fluid flow. In some cases, the angle θ₁ is from about 3° to about 30° relative to the direction of the fluid flow. In some cases, the angle θ₂ is from about 2° to about 90°. In some cases, the angle θ₂ is from about 5° to about 50°. In some cases, a first obstacle of the first line of the first obstacles and a second obstacle of the first line of the first obstacles are separated by an inter-obstacle distance in a plane defined by the x-axis and the y-axis. In some cases, the inter-obstacle distance is based at least in part on a dimension of a target analyte. In some cases, the inter-obstacle distance is at least about 1, 2, 3, 4, 5, 6, 7, 8, 9, 10, 11, 12, 13, 14, 15, 16, 17, 18, 19, or 20 micrometers (μm). In some cases, the first vertical spacing is greater than or less than a threshold value based on a dimension of a first target analyte. In some cases, the first vertical spacing is greater than about 8 micrometers (μm). In some cases, the first vertical spacing is less than about 6 μm. In some cases, the first vertical spacing is from about 5 μm to about 12 μm. In some cases, the fluidic device further comprises a second array of obstacles disposed in the second side channel, wherein the second side channel comprises: another inlet fluidically connected to the outlet of the main channel; and another outlet; a second length defining a second x-axis direction; a second width defining a second y-axis direction a second height defining a second z-axis direction; wherein the second array of obstacles is disposed in the second side channel, extending from the ceiling and toward the surface of the substate substantially along the second z-axis, wherein each second obstacle comprises a second length along the z-axis, and wherein the second length is shorter than the second height of the side channel, thereby separating each second obstacles from the surface of the substrate by a second vertical spacing. The fluidic device of claim 17, wherein the second vertical spacing is based at least in part on a dimension of a second target analyte. In some cases, the second vertical spacing is less than the first vertical spacing. In some cases, the second vertical spacing is greater than the first vertical spacing. In some cases, the second vertical spacing is substantially the same as the first vertical spacing. In some cases, the fluidic device further comprises a fluidic component in fluidic communication with an outlet of the first side channel or an outlet of the second side channel and the inlet of the main channel.

In some aspects, the present disclosure provides for a system for separating a plurality of biological particles comprising: a substrate; a fluidic channel disposed in or along the substrate, wherein the fluidic channel comprises: a top surface defining a ceiling of the fluidic channel opposing a surface of the substrate; a main channel comprising: an inlet; an outlet; a length defining an x-axis direction; a width defining a y-axis direction; a height defining a z-axis direction; a first side channel connected to the outlet of the main channel; a second side channel connected to the outlet of the main channel; an array of first obstacles disposed in the main channel, extending from the ceiling and toward the surface of the substrate substantially along the z-axis, wherein each first obstacle comprises a first length along the z-axis, and wherein the first length is shorter than the height of the main channel, thereby separating the each first obstacle from the surface of the substrate by a first vertical spacing; and a fluid flow module configured to: direct a fluid stream through the fluidic channel to separate one or more particles from the fluid stream using the array of first obstacles.

In some cases, the array of the first obstacles comprises at least a first line of the first obstacles and a second line of the first obstacles, wherein the first line of the first obstacles is separated from the second line of the first obstacles by at least a first distance along the y-axis. In some cases, the first distance along the y-axis is at least about 10 nanometers (nm), 20 nm, 40 nm, 50 nm, 60 nm, 70 nm, 80 nm, 90 nm, 100 nm, 200 nm, 300 nm, 500 nm, 1 micrometer (μm), 2 μm, 5 μm, 10 μm, 30 μm, 50 μm, or 100 μm. In some cases, the array of the first line of the first obstacles is at an angle of θ₁ relative to the x-axis on a plane defined by the x-axis and the y-axis. In some cases, the first line of the first obstacles comprises a substantially similar parallelogram cross-section on the plane, wherein the parallelogram comprises an acute angle of θ₂, and wherein θ₂ is larger than θ₁. In some cases, the angle θ₁ is from about 1° to about 85° relative to the direction of the fluid flow. In some cases, the angle θ₁ is from about 3° to about 30° relative to the direction of the fluid flow. In some cases, the angle θ₂ is from about 2° to about 90°. In some cases, the angle θ₂ is from about 5° to about 50°. In some cases, a first obstacle of the first line of the first obstacles and a second obstacle of the first line of the first obstacles are separated by an inter-obstacle distance in a plane defined by the x-axis and the y-axis. In some cases, the inter-obstacle distance is based at least in part on a dimension of a target analyte. In some cases, the inter-obstacle distance is at least about 1, 2, 3, 4, 5, 6, 7, 8, 9, 0, 11, 12, 13, 14, 15, 16, 17, 18, 19, or 20 micrometers (μm). In some cases, the first vertical spacing is greater than or less than a threshold value based on a dimension of a first target analyte. In some cases, the first vertical spacing is greater than about 8 micrometers (μm). In some cases, the first vertical spacing is less than about 6 μm. In some cases, the first vertical spacing is from about 5 μm to about 12 μm. In some cases, the system further comprises a second array of obstacles disposed in the second side channel, wherein the second side channel comprises: another inlet fluidically connected to the outlet of the main channel; and another outlet; a second length defining a second x-axis direction; a second width defining a second y-axis direction a second height defining a second z-axis direction; wherein the second array of obstacles is disposed in the second side channel, extending from the ceiling and toward the surface of the substate substantially along the second z-axis, wherein each second obstacle comprises a second length along the z-axis, and wherein the second length is shorter than the second height of the side channel, thereby separating each second obstacles from the surface of the substrate by a second vertical spacing. In some cases, the second vertical spacing is based at least in part on a dimension of a second target analyte. In some cases, the second vertical spacing is less than the first vertical spacing. In some cases, the second vertical spacing is greater than the first vertical spacing. In some cases, the second vertical spacing is substantially the same as the first vertical spacing. In some cases, the particles comprise cells. In some cases, the particles comprise senescent cells. In some cases, the particles comprise necrotic cells. In some cases, the particles comprise viable cells, senescent cells, and necrotic cells. In some cases, the fluid flow module comprises a pump. In some cases, the pump comprises a peristaltic pump. In some cases, the system further comprises a fluidic connection between an outlet of the first side channel or the second side channel and the inlet of the main channel. In some cases, the fluid flow module is further configured to recycle at least a portion of the fluid stream to the fluidic channel.

In some aspects, the present disclosure provides for a method, comprising: directing a fluid comprising a plurality of particles into a microfluidic device, the microfluidic device comprising: a substrate; a fluidic channel disposed in or along the substrate, wherein the fluidic channel comprises: a top surface defining a ceiling of the fluidic channel opposing a surface of the substrate; a main channel comprising: an inlet; an outlet; a length defining an x-axis direction; a width defining a y-axis direction; a height defining a z-axis direction; a first side channel connected to the outlet of the main channel; a second side channel connected to the outlet of the main channel; and an array of first obstacles disposed in the main channel, extending from the ceiling and toward the surface of the substrate substantially along the z-axis, wherein each first obstacle comprises a first length along the z-axis, and wherein the first length is shorter than the height of the main channel, thereby separating the each first obstacle from the surface of the substrate by a first vertical spacing; directing the fluid through the fluidic channel; and separating a first portion of the plurality of particles from the fluid using the array of first obstacles upon flow of the fluid through the array of first obstacles. In some cases, the array of the first obstacles comprises at least a first line of the first obstacles and a second line of the first obstacles, wherein the first line of the first obstacles is separated from the second line of the first obstacles by at least a first distance along the y-axis. In some cases, the first distance along the y-axis is at least about 10 nanometers (nm), 20 nm, 40 nm, 50 nm, 60 nm, 70 nm, 80 nm, 90 nm, 100 nm, 200 nm, 300 nm, 500 nm, 1 micrometer (μm), 2 μm, 5 μm, 10 μm, 30 μm, 50 μm, or 100 μm. In some cases, the array of the first line of the first obstacles is at an angle of θ₁ relative to the x-axis on a plane defined by the x-axis and the y-axis. In some cases, the first line of the first obstacles comprises a substantially similar parallelogram cross-section on the plane, wherein the parallelogram comprises an acute angle of θ₂, and wherein θ₂ is larger than θ₁. In some cases, the angle θ₁ is from about 1° to about 85° relative to the direction of the fluid flow. In some cases, the angle θ₁ is from about 3° to about 30° relative to the direction of the fluid flow. In some cases, the angle θ₂ is from about 2° to about 90°. In some cases, the angle θ₂ is from about 5° to about 50°. In some cases, a first obstacle of the first line of the first obstacles and a second obstacle of the first line of the first obstacles are separated by an inter-obstacle distance in a plane defined by the x-axis and the y-axis. In some cases, the inter-obstacle distance is based at least in part on a dimension of a target analyte. In some cases, the inter-obstacle distance is at least about 1, 2, 3, 4, 5, 6, 7, 8, 9, 10, 11, 12, 13, 14, 15, 16, 17, 18, 19, or 20 micrometers (μm). In some cases, the first vertical spacing is greater than about 8 micrometers (μm). In some cases, the first vertical spacing is less than about 6 μm. In some cases, the first vertical spacing is from about 5 μm to about 12 μm. In some cases, the method further comprises recycling at least a portion of the fluid stream to repeat (a)-(c) one or more times. In some cases, the at least the portion of the fluid stream comprises a reduced amount of a particle of the plurality of particles. In some cases, the second side channel comprises: another inlet fluidically connected to the outlet of the main channel; and another outlet; a second length defining a second x-axis direction; a second width defining a second y-axis direction a second height defining a second z-axis direction; wherein the second array of obstacles is disposed in the second side channel, extending from the ceiling and toward the surface of the substate substantially along the second z-axis, wherein each second obstacle comprises a second length along the z-axis, and wherein the second length is shorter than the second height of the side channel, thereby separating each second obstacles from the surface of the substrate by a second vertical spacing. In some cases, the fluid comprises a biofluid. In some cases, the biofluid comprises whole blood, plasma, or serum. In some cases, the whole blood is undiluted or diluted. In some cases, the plurality of particles comprises cells. In some cases, the cells comprise senescent cells. In some cases, the cells comprise necrotic cells. In some cases, the cells comprise viable cells, senescent cells, and necrotic cells. In some cases, the first vertical spacing is based at least in part on a dimension of a particle of the plurality of particles. In some cases, the first vertical spacing is less than or equal to a threshold value based on the dimension of the particle. In some cases, the second vertical spacing is based at least in part on a dimension of a second particle of the plurality of particles. In some cases, the second vertical spacing is less than or equal to a threshold value based on the dimension of the particle. In some cases, wherein the second vertical spacing is less than the first vertical spacing. In some cases, the second vertical spacing is greater than the first vertical spacing. In some cases, the second vertical spacing is substantially the same as the first vertical spacing.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1C illustrate sample chips and uses thereof (e.g., for processing of senescent cells). FIG. 1A shows two types of sample chips: (i) a chip with a 3D filter array and a cell trap array for capture and single cell analysis of senescent cells in blood; and (ii) a chip with a 3D filter array connected with a tubing at the outlet for removal of senescent cells from blood. Zoom-in regions show schematic separation and trapping of red blood cell (RBC), while blood cell (WBC), and senescent cells, along with two types of pillar shapes A and B. FIG. 1B shows mechanism of size-based cell separation with a 3D filter array: (i) the top view of the filters with force analysis on the x and y directions; (ii) the side view of the filters on the x and z directions; and (iii) the perspective view of the filters on the x, y, and z directions. FIG. 1C shows images of the experimental setup and operation: (i) an actual-size image of a chip relative to a US dime; (ii) the experimental setup showing tubing connections and pumps; and (iii) a chip in operation of processing whole blood samples. Scale bar represents 5 mm in (iii);

FIGS. 2A-2C illustrate operation of sample chips. FIG. 2A shows time-lapse images of (i) a bead and (ii) a cell, roll down on a 3D filter array. FIG. 2B shows (i) images of undiluted whole blood passing through a 3D-filter array without clogging (ii) stacking images showing complete separation of 18 μm beads from 10 μm beads (left), and separation of MSCs from undiluted whole blood (right). FIG. 2C shows images of cell trap array located at (i) blood outlet and (ii) senescent cell outlet, after separation of MSCs from whole blood. Cells with blue color are senescent cells (SA-β-gal positive); (iii) phase-contrast and fluorescence imaging of CD45 labeling for identification of senescent MSCs and WBCs. Scale bars represent 50 μm in FIG. 2A and FIG. 2C, 150 μm in (FIG. 2B-i), and 100 μm in (FIG. 2B-ii), respectively. FIGS. 2A-2C are shown as the corresponding zoom-in regions on a sample schematic chip for presentation clarity;

FIGS. 3A-3D show validation of sample chips for size-based separation. FIG. 3A shows schematic of a sample chip for characterization with beads or cells. FIG. 3B shows recovery of beads from outlet (iii), for four sizes of beads (6 μm, 10 μm, 15 μm, and 18 μm) mixed to characterize three types of chips (z-direction only filter, 4 μm 3D filter, and 13 μm 3D filter) at three flow rates (1 mL/h, 3 mL/h, and 5 mL/h). FIG. 3C shows recovery of WBCs isolated from whole blood from outlet (iii), with three types of chips at three flow rates as in FIG. 3B. FIG. 3D shows recovery of basal MSCs from undiluted whole blood at outlet (iv), with three types of chips at a flow rate of 3 mL/h;

FIGS. 4A-4C show application of sample chips for analysis of senescent cells in biofluids. FIG. 4A shows senescence-associated beta-galactosidase (SA-β-gal) staining of MSCs cultured on a 12-well plate. The MSCs are treated with different doses of hydrogen peroxide (H₂O₂, 0, 100, 200 μM) and X-ray (0, 1, 4 Gy), and analyzed 3 days and 6 days after the treatments. Cells stained blue are SA-β-gal positive. FIG. 4B shows quantitation of SA-β-gal staining of MSCs on culture dish (i) and MSCs isolated from human whole blood on the chip (ii). The percentage of SA-β-gal positive is calculated for the stained blue-stained MSCs among the total MSCs. FIG. 4C shows isolation and analysis of senescent cells from mouse bone marrow after total body irradiation (TBI) of 0, 1 Gy, 4 Gy, and 6 Gy X-ray radiation (n=3), respectively. Scale bar represents 100 μm in FIG. 4A;

FIGS. 5A-5D show application of sample chips for removal of senescent cells from whole blood. FIG. 5A shows schematic of removal of senescent cells from whole blood, using a 13 μm 3D filter. FIG. 5B shows cell size distribution of basal MSCs and senescent MSCs, 3 and 6 days after treatment with different doses of hydrogen peroxide and X-ray. FIG. 5C illustrates enrichment of senescent cells at outlet (iv) using a 13 μm 3D filter chip. In comparison, original MSCs without separation, MSCs directly captured on a cell trap array without a 3D filter, and MSCs processed on a chip with a 4 μm 3D filter are also studied. FIG. 5D shows removal of senescent cells from undiluted whole blood using a 13 μm 3D filter chip via outlet (iv);

FIGS. 6A-6E show sample ultrahigh-throughput chips for removal of senescent cells from human whole blood. FIG. 6A shows schematic of the high-throughput chip. Five large-dimension channels are stacked and integrated for parallel processing. FIG. 6B shows image of a sample high-throughput chip compared to a regular-size single-unit device. FIG. 6C shows cross-section view of the multi-layer and multi-channel chip showing integration of five channels in five vertical layers. FIG. 6D shows microscope images showing MSCs before separation, spiked in blood, and after separation. FIG. 6E shows quantification of the numbers of basal MSCs and senescent MSCs spiked into whole blood, before and after removal of senescent MSCs using our chip. Scale bars represent 10 mm in FIG. 6B and 50 μm in FIG. 6D;

FIGS. 7A-7D show flow simulation inside a microfluidic channel. FIG. 7A shows simulation of flow velocity in a 2D filter array. FIG. 7B shows simulation of flow velocity in a 3D filter array. FIG. 7C shows plot of flow velocity along the dash line in FIG. 7A. FIG. 7D shows plot of flow velocity along the dash line in FIG. 7B; and

FIGS. 8A-8D illustrate sample chips, sample fabrication, and sample performance thereof (e.g., for processing of a mixture of live, senescent, and necrotic cells). FIG. 8A shows a sample device for separating a mixture of analytes. Zoom-in regions show schematic obstacle shapes and arrangements. FIG. 8B shows schematics of example devices of the present disclosure with one or two obstacle arrays for separating heterogenous mixtures of analytes. FIG. 8C depicts cross-sections of the device showing filter features and chip channel heights of the example chips. The z-gap and the channel height for each device are listed in the table. FIG. 8D summarizes performance outcomes of the example devices;

FIG. 9 illustrates a representative fabrication process for devices as described herein;

FIG. 10 shows cell diameter measures of mouse mesenchymal stem cells (mMSCs) at outlets of an example device described herein;

FIG. 11 shows a plot of cellular senescence ratio of human mesenchymal stem cells at 1(circles), 3 (diamonds), and 7 (triangles) days after treatment with 100, 200, or 300 μM hydrogen peroxide;

FIG. 12 summarizes measures cellular senescence in mMSCs. Panel (a) depicts the senescence ratio of mMSCs treated with hydrogen peroxide (H₂O₂) at 150 μM (diamonds) or control (circles) for three hours at 1, 2, 5 and 8 days after treatment. Panel (b) depicts the senescence ratio of mMSCs exposed to 0 (basal), 1, 4 and 6 Gy X-rays at 1, 4, 7 and 11 days after exposure;

FIG. 13 illustrates necrosis induction in hMSCs;

FIG. 14A shows a diagram of a cyclic fluidic system in an example of systems of the present disclosure. FIG. 14B shows pressure and temperature readings of sensors of the system depicted in FIG. 14A over an 800-second continuous flow cycle;

FIG. 15 shows representative images of mouse MSCs stained with Hoechst, anti-scal, anti-CD140a, and anti-CD-117;

FIGS. 16A and 16B show representative images of Hoechst, anti-sca1, anti-CD140a channels, and combined channel images of MSCs from sham irradiated mice (FIG. 16A) and MSCs from 6.5 Gy irradiated mice (FIG. 16B);

FIGS. 17A and 17B show representative images of Hoechst, anti-sca1, anti-CD140a channels, and combined channel images of HSCs from sham irradiated mice (FIG. 17A) and HSCs from 6.5 Gy irradiated mice (FIG. 17B);

FIG. 18A illustrates an example device of the present disclosure with two arrays of obstacles for separating target analytes (top), time laps images showing movement of analytes through the device (bottom), and representative images of hMSCs acquire from outlet (i) (bottom right). FIG. 18B shows the percentage of necrotic hMSCs in the sample input and each outlet of the example device depicted in FIG. 18A. The input hMSCs were treated with 300 μM H₂O₂ for 3 hours. FIG. 18C shows the percentage of necrotic, senescent, and viable hMSCs in the sample input and each outlet of the example device of FIG. 18A. The input hMSCs were a mixture of basal and H₂O₂ treated hMSCs;

FIG. 19A illustrates an example system of the present disclosure for cycling a fluid through a device multiple times. the separation sequence of hMSCs using the 2-stage device, where the 2^{nd}and 3^{rd} sample inputs were the solutions collected from the outlet (ii) in the preceding cycle, respectively. FIG. 19B illustrates the cell input into the system; a mixture of basal (38%) and H₂O₂ (62%) hMSCs. The left panel shows the size distribution of basal or H₂O₂ (300 μM, 3 hours) treated hMSCs. The right panel shows the necrotic, senescent, and viable cell percentage in basal or H₂O₂ hMSCs. FIG. 19C illustrates the cell size distribution in the initial cell input, in outlets (i)-(iii) after the 1^{st} run of separation, in outlets (i)-(iii) after the 2^{nd} run of separation, and in outlets (i)-(iii) after the 3^{rd} run of separation. FIG. 19D shows the necrotic (top), senescent (middle), and viable hMSC percentage (bottom) in the sample input, outlet (i)-(iii) following the 1^{st}, 2^{nd}, and 3^{rd} run, respectively;

FIGS. 20A-20E summarize the separation performance of the system of FIG. 20A at separating mMSCs. FIG. 20A illustrates the size distribution of H₂O₂ (150 μM, left) treated or X-ray (6 Gy, right) radiated mMSCs from the sample input and each outlet. FIG. 20B shows the purity of mMSCs>15 μm at input, outlet (i), outlet (ii), and outlet (iii). mMSCs were either treated with H₂O₂ (circle) or exposed to X-ray radiation (diamond). FIG. 20C illustrates the morphologies of basal mMSCs collected in the sample input, outlet (i), and outlet (ii) and post-cultured for 7 days. The left panel of FIG. 20D shows the percentage of basal mMSCs in the sample input, outlet (i), and outlet (ii). The right panel of FIG. 20D shows the doubling time of basal mMSCs in the sample input, outlet (i), and outlet (ii). The top panel of FIG. 20E shows an illustration of a separation sequence of mMSCs through an example cyclic fluidic system of the present disclosure, where outlet (i) connects with buffer inlet while outlet (ii) & (iii) connect with sample input. the 2^{nd} and 3^{rd} cell inputs are the solutions collected from the outlet (ii) of the 1^{st} and 2^{nd} cycle, respectively. The bottom left panel of FIG. 20E shows the percentage mMSCs in the outlet (i), outlet (ii)+(iii), and the recovery rate of mMSCs after 990 seconds of cycling operation. The bottom right panel of FIG. 20E show the size distribution of H₂O₂ (150 μM,) treated mMSCs from the sample input (left box plot), outlet (i) (middle box plot), outlet (ii)+(iii) (right box plot). The mMSC in the sample input is the mixture of basal and H₂O₂ (150 μM) treated mMSCs;

FIGS. 21A-21F summarize separation performance of mouse bone marrow samples through an example device of the present disclosure. FIG. 21A depicts an image showing the staining of bone marrow-derived MSCs and HSCs from sham and 6.5 Gy irradiated mice 6 days after radiation exposure. The scale bar is 50 μm. FIG. 21B shows the size distribution of bone marrow-derived MSCs and HSCs from sham and 6.5 Gy irradiated mice. FIG. 21C shows the purity of cells >10 μm in input, outlet (i), outlet (ii), and outlet (iii) for sham (circle) and X-ray irradiated mice (diamond). FIG. 21D shows the size distribution of bone marrow-derived cells collected from the input (leftmost box plot), outlet (i) (second box plot from left), outlet (ii) (second box plot from right), and outlet (iii) (rightmost box plot) from sham and 6.5 Gy irradiated mice. FIG. 21E shows the SA-β-gal⁺ cell fraction (representative cells indicated with an arrow) in the bone marrow of sham or 6.5 Gy irradiated mice (left). The scale bar is 20 μm. The average size of senescent cells found in the bone marrow of sham or 6.5 Gy irradiated mice (right). FIG. 21F shows the senescence cell ratio in the input, outlet (i), outlet (ii), and outlet (iii) for sham (circle) and X-ray irradiated mice (diamond); and

FIG. 22 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “obstacle,” as used herein, generally refers to any structure that is capable of obstructing a flow of a fluid, impeding the flow of the fluid, and/or diverting the flow of the fluid. In some examples, the obstacle is pillar. The pillar may have dimensions on the order of nanometers (i.e., nanopillar) or micrometers (i.e., micropillar). The obstacle may be distributed in an array of a plurality of obstacles (or array of obstacles). The obstacle may have various shapes. The obstacle may have a cross-section that is circular, triangular, quadrilateral, pentagonal, hexagonal, or any combination of shapes or partial-shapes thereof. In some cases, the obstacles may have a parallelogram cross-section. In some cases, the parallelogram may have a certain interior angle. In some cases, the interior angel may range from about 1° to about 90°, such as about 5° to 50°.

An array of obstacles may comprise a plurality of obstacles that have regular or substantially regular shapes and/or sizes. In some examples, the plurality of obstacles have a coefficient of variation of less than or equal to about 30%, 25%, 20%, 15%, 10%, 5%, 4%, 3%, 2%, 1%, or less. As an alternative, the plurality of obstacles may have irregular or substantially irregular shapes and/or sizes.

In some examples, the plurality of obstacles is generally distributed in an array that is angled with respect to the general direction of flow into the array. Such array may not include other obstacles. For example, the plurality of obstacles are oriented at an angle greater than about 0°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85° or more with respect to the general direction of flow into the array (e.g., a vector parallel to one or more axes directed through one or more subsets of the plurality of particles may be oriented at an angle greater than 0° with respect to a vector oriented along the general direction of flow). Such angle may be constant with respect to the general direction of flow. Alternatively, such angle may vary along the general direction of flow (e.g., the angle may increase or decrease along the general direction of flow).

Flow directed in an array of obstacles may be laminar. As an alternative, the flow may be turbulent.

Whenever the term “at least,” “greater than,” or “greater than or equal to” precedes the first numerical value in a series of two or more numerical values, the term “at least” or “greater than” applies to each one of the numerical values in that series of numerical values.

Whenever the term “no more than,” “less than,” or “less than or equal to” precedes the first numerical value in a series of two or more numerical values, the term “no more than” or “less than” applies to each one of the numerical values in that series of numerical values.

Provided herein are methods and systems for separating, isolating, capturing, detecting and/or analyzing target analytes. The target analytes may comprise biological particles. Biological particles may include any particles of biological origin. Non-limiting examples of biological particles may include cells or components thereof, viruses, bacteria, proteins, carbohydrates, nucleic acid molecules (such as deoxyribonucleic acid (DNA), ribonucleic acid (RNA)), lipid, or combinations thereof. Non-limiting examples of cells may include, tumor cells, red blood cells, white blood cells (such as T cells, B cells, and helper T cells), infected cells, trophoblasts, fibroblasts, stem cells, epithelial cells, infectious organisms (e.g., bacteria, protozoa, and fungi), cancer cells, bone marrow cells, fetal cells, progenitor cells, foam cells, mesenchymal cells, immune system cells, endothelial cells, endometrial cells, connective tissue cells, trophoblasts, bacteria, fungi, or pathogens, or combinations thereof. In some cases, cells may comprise senescent cells. Senescent cells may comprise senescent cells of any type of above-mentioned cells. For example, senescent cells may comprise senescent T cells, senescent white blood cells, senescent microphages, senescent lung, breast, colon, prostate, gastric, hepatic, ovarian, esophageal, or bronchial epithelial or stromal cells, senescent skin epithelial or stromal cells, senescent glial cells, senescent vascular endothelial or stromal cells, or combinations thereof.

### Systems

Systems of the present disclosure may comprise microfluidic devices. A microfluidic device, as provided herein, may comprise a body structure. The body structure may be a single layer or multi-layer structure. The body structure may comprise a substrate. The substrate may comprise a fluidic channel disposed therein. The fluidic channel may have an aspect ratio (a ratio of channel length to an average cross-sectional dimension of the channel) that is greater than or equal to about 2, 3, 4, 5, 6, 7, 8, 9, 10, 11, 12, 13, 14, 15, 16, 17, 18, 19, 20, 22, 24, 26, 28, 30, 32, 34, 36, 38, 40, 45, 50, 55, 60, 65, 70, 75, 80, 85, 90, 95, 100, or more. In some cases, the aspect ratio may be less than or equal to about 200, 180, 160, 140, 120, 100, 80, 70, 60, 50, 40, 30, 20 or less. In some cases, the aspect ratio may be between any of the two values described above and elsewhere herein, for example, from about 15 to 30.

The fluidic channel may comprise one or more obstacles disposed therein. The one or more obstacles may be a plurality of obstacles. The obstacles may be any structures that may have an impact or effect on a fluid or components thereof, while the fluid flows through the microfluidic channel. For example, the obstacles may delay, alter, or impede a fluid flow (e.g., flow rate of the fluid flow) in the channel. The obstacles may comprise obstacles associated with or immobilized on a surface (e.g., bottom, top or side walls) of the microfluidic channel. The surface may be a substrate or a side wall of the fluidic channel. The obstacles may be extended partially or fully across the channel. The obstacles may be extended partially or fully along a height of the fluidic channel. The obstacles may have an average height that is less than or equal to an average height (or depth) of the microfluidic channel. The obstacles may have an average height that is greater than or equal to about 1 micrometer (micron, μm), 2 μm, 5 μm, 10 μm, 12 μm, 14 μm, 16 μm, 18 μm, 20 μm, 22 μm, 24 μm, 26 μm, 28 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, or more. The obstacles may have an average height that is less than or equal to about 150 μm, 125 μm, 100 μm, 90 μm, 80 μm, 70 μm, 60 μm, 50 μm, 45 μm, 40 μm, 35 μm, 30 μm, 25 μm, 20 μm, 15 μm, 10 μm, 8 μm, 6 μm, 4 μm, 1 μm, or less. In some cases, the obstacles have an average size that falls between any of the two values described above or elsewhere herein, for example, from about 30 μm to 35 μm.

The obstacles may have a vertical spacing (or z-gap) between a bottom surface of the obstacles and a surface (e.g., bottom surface or substrate) of a fluidic channel. The obstacles may extend from a top surface defining a ceiling of the fluidic channel. The top surface may oppose a surface of the substrate. The vertical spacing may be in a direction which is perpendicular to a plane of a substate in which the fluidic channel is disposed. In an example, the fluidic channel comprises a length defining an x-axis, a width defining a y-axis, and a heigh defining a z-axis. The height (and vertical spacing) is along the z-axis which is perpendicular to a plane defined by the x-axis and the y-axis. The obstacles may have a length along the z-axis, which is shorter than the height of the channel, thereby separating each obstacle from the surface of the substrate by the vertical spacing (z-gap). The vertical spacing may allow for separation of one or more distinct types or kinds (e.g., having a characteristic dimension or characteristic biological state or function) of particles from heterogenous mixtures of particles comprised in the same fluid. In an example, particles having a characteristic dimension no more than a threshold value (e.g., less than about the vertical spacing or less) may pass through the array of obstacles unimpeded while particles having a corresponding dimension larger than the cutoff value may be deflected, thus allowing separation based on the characteristic dimension. The vertical spacing may comprise a distance that is greater than or equal to about 1 micrometer (micron, μm), 2 μm, 5 μm, 10 μm, 12 μm, 14 μm, 16 μm, 18 μm, 20 μm, 22 μm, 24 μm, 26 μm, 28 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, or more. The vertical spacing may comprise an average height that is less than or equal to about 150 μm, 125 μm, 100 μm, 90 μm, 80 μm, 70 μm, 60 μm, 50 μm, 45 μm, 40 μm, 35 μm, 30 μm, 25 μm, 20 μm, 15 μm, 10 μm, 8 μm, 6 μm, 4 μm, 1 μm, or less. In some cases, the obstacles have an average size that falls between any of the two values described above or elsewhere herein, for example, from about 5 μm to 12 μm.

The obstacles may be microstructures, nanostructures, or combinations thereof. The obstacles may be three-dimensional (3D) structures. The 3D obstacles may be obstacles that have openings in x-, y-, and z-directions. The 3D obstacles may deform in x-, y-, and/or z-directions upon application of a pressure. The pressure may be resulted from a fluid flow. The pressure may change with flow rate of the fluid flow. The obstacles may comprise micropillars. The micropillars may be 3D micropillars. The obstacles may have an average size that is greater than or equal to about 1 μm, 2 μm, 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, or more. The obstacles may have an average size that is less than or equal to about 200 μm, 180 μm, 160 μm, 140 μm, 120 μm, 100 μm, 90 μm, 80 μm, 60 μm, 50 μm, 40 μm, 30 μm, 20 μm, 15 μm, 10 μm, 8 μm, 6 μm, 4 μm, 2 μm, 1 μm, or less. In some cases, the obstacles may have an average size that is between any of the two values described above and elsewhere herein, for example, from about 1 μm to 100 μm.

The obstacles may be porous or nonporous. The obstacles may be solid, or semi-solid. Materials suitable for forming the obstacles may include polymers, metals, ceramics, carbons, or combinations thereof.

The dimensions and geometry of the obstacles may vary. The obstacles may have regular, or irregular cross sections. In some cases, the obstacles comprise one or more subsets of the obstacles. The one or more subsets of the obstacles may comprise obstacles having cross sections that are the same as or different from one another. In some cases, the obstacles have quadrilateral cross sections such as parallelogram cross sections. In some cases, the parallel cross sections comprise an acute angle. The acute angle may be any angle less than about 90°, such as, for example, about 5° to about 50°.

In some cases, at least a subset of the obstacles may be slanted. The subset of the obstacles may be slanted in vertical direction. The subset of the obstacles may be slanted in vertical direction that is perpendicular to a plane of a substrate within which a microfluidic channel is disposed. The subset of the obstacles may be slanted in various angular directions. The various angular directions may be any directions that are angled with respect to, e.g., a plane of a substrate within which a microfluidic channel is disposed. The angle may be from about 0° to 90°. For example, the angle may be from about 1° and about 85° or about 5° and about 50°. In some cases, the subset of obstacles may be slanted at an angle smaller than an angle of a parallelogram cross-section of the subset of obstacles. In an example, the angle of the parallelogram cross-section may be about 30° and the angle of the array of obstacles may be about 4°.

The one or more obstacles may comprise an array of obstacles. The array of obstacles may comprise any number of obstacles (e.g., greater than or equal to about 5, 10, 15, 20, 25, 30, 35, 40, 45, 50, 55, 60, 65, 70, 75, 80, 85, 90, 95, 100, 1000, or more obstacles). The array of obstacles may be angled relative to a direction of a fluid flow in the microfluidic channel. The array of obstacles may be aligned or oriented to a direction that is angled relative to the direction of the fluid flow. There may be an angle between the direction along which the array of obstacles is aligned and the direction of the fluid flow. The angle may be an oblique angle. The angle may be from about 0° to about 90°. In some cases, the angle may be greater than about 0°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85° or more. In some cases, the angle may less than about 90°, 85°, 80°, 75°, 70°, 65°, 60°, 55°, 50°, 45°, 40°, 35°, 30°, 25°, 20°, 15°, 10°, 9°, 7°, 5°, 3°, 1°, or less. In some cases, the angle may be between any of the values described above or elsewhere herein, for example, from about 20° to 30°. In some cases, all of the obstacles are angled relative to the direction of the fluid flow. In some cases, the obstacles are angled at an angle less than an angle of a parallelogram cross section of one or more of the obstacles.

The obstacles may be spaced from one another. An average spacing size of the obstacles (e.g., an average space between adjacent obstacles) may vary. The average spacing size may be adjusted depending upon a variety of factors, including such as dimension of the microfluidic channel, number of obstacles disposed in the microfluidic channel, sample volume, sizes, dimensions, geometries of target analytes, fluid flow rate, or combinations thereof. In some cases, the obstacles may have an average spacing size greater than or equal to about 10 nanometers (nm), 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 5 μm, 10 μm, 20 μm, 30 μm, 40 μm, 50 μm, 60 μm, 70 μm, 80 μm, 90 μm, 100 μm, or more. In some cases, the average spacing size may be less than or equal to about 200 μm, 180 μm, 160 μm, 140 μm, 120 μm, 100 μm, 85 μm, 75 μm, 65 μm, 55 μm, 45 μm, 35 μm, 25 μm, 15 μm, 5 μm, 1 μm, 850 nm, 750 nm, 650 nm, 550 nm, 450 nm, 350 nm, 250 nm, 150 nm, 100 nm, or less. In some cases, the average spacing size may be any of the values described above or elsewhere herein, for example, from about 100 nm to 100 μm.

The array of obstacles may comprise a line of obstacles. A first obstacle of the array of obstacles and a second obstacle of the line of obstacles may be separated by an inter-obstacle distance. The inter-obstacle distance may comprise a longest or shortest distance between opposing faces of each obstacle. The inter-obstacle distance may be at least about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, or more. The inter-obstacle distance may be at most about 20 μm, 19 μm, 18 μm, 17 μm, 16 μm, 15 μm, 14 μm, 13 μm, 12 μm, 11 μm, 10 μm, 9 μm, 8 μm, 7 μm, 6 μm, 5 μm, 4 μm, 3 μm, 2 μm, 1 μm, or less. The inter-obstacle distance may vary between successive obstacles of the line of obstacles. Alternatively, the inter-obstacle distance may be the same between each pair of adjacent obstacles in the line of obstacles. The array may comprise a plurality of lines of obstacles. The lines of obstacles may be separated by a distance (e.g., a distance in a direction of the y-axis or x-axis of the channel). The distance may be the same between each successive line of obstacles. Alternatively, the distance may vary between each successive line of obstacles. In some cases, the distance between the lines of obstacles may have an average distance equal to about 10 nanometers (nm), 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 5 μm, 10 μm, 20 μm, 30 μm, 40 μm, 50 μm, 60 μm, 70 μm, 80 μm, 90 μm, 100 μm, or more. In some cases, the average distance may be less than or equal to about 200 μm, 180 μm, 160 μm, 140 μm, 120 μm, 100 μm, 85 μm, 75 μm, 65 μm, 55 μm, 45 μm, 35 μm, 25 μm, 15 μm, 5 μm, 1 μm, 850 nm, 750 nm, 650 nm, 550 nm, 450 nm, 350 nm, 250 nm, 150 nm, 100 nm, or less. In some cases, the average distance may be any of the values described above or elsewhere herein, for example, from about 100 nm to 100 μm.

The inter-obstacle distance may be configured to sort or isolate one or more particles based on the size of the inter-obstacle distance. Particles with a dimension (e.g., diameter) greater than a threshold length may roll down an array of obstacles with a corresponding inter-obstacle distance while those particles with a dimension (e.g., diameter) smaller than the threshold length may pass through the inter-obstacle spaces. Accordingly, particles greater than or equal to the threshold distance may be deflected to a separate portion of the device or system (e.g., a first or second side channel in fluidic communication with a main channel comprising the array of obstacles characterized by the inter-obstacle spacing) or isolation.

The one or more obstacles may comprise a plurality of arrays of obstacles. In some cases, the microfluidic channel may have a uniform cross sectional dimension, and the plurality of the obstacle arrays may be disposed within the microfluidic channel. In some cases, the microfluidic channel may comprise one or more sections along a length of the channel. The one or more sections may have the same or different cross sectional dimensions. At least one obstacle array may be disposed within each section of the microfluidic channels. The obstacle array disposed in different sections of the microfluidic channel may be the same or may be different. The obstacle array disposed in different sections of the microfluidic channel may comprise obstacles that are of the same or different sizes, shapes, geometries, and/or cross-sections. For example, a microfluidic channel may comprise at least two sections each comprising an array of obstacles. The obstacle arrays disposed in different sections may comprise different number of obstacles. Each array may have a distinctive size and geometry (i.e., obstacles comprised in one array may have a size and/or cross section different from those comprised in the other array). The two sections may each be configured to separate or isolate a specific type of target analyte comprised in a fluid while the fluid is flowing through the microfluidic channel. In another example, a main fluidic channel may be in fluidic configuration with a first side channel and a second side channel. The first side channel or the second side channel may have an array of obstacles disposed within it.

In some cases, the microfluidic channel may comprise a plurality of sections (e.g., a main fluidic channel and one or more side channels) each comprising an array of obstacles with a different vertical spacing or inter-obstacle distance. Each vertical spacing or inter-obstacle distance may be configured to sort a particular kind or type of target analytes (e.g., having a particular characteristic dimension or biological classification). Analytes having a dimension less than a cutoff value based on the vertical spacing or inter-obstacle distance may pass through the obstacles while those having a dimension greater than or equal to the cutoff value may be deflected by the obstacles, allowing for separation. In some cases, the vertical spacing or inter-obstacle distance of each array of obstacles may be the same or substantially the same.

In some cases, the microfluidic device comprises a plurality of fluidic channels. For at least a subset of the plurality of the fluidic channels, each individual fluidic channel may comprise a different array of obstacles disposed therein. The different obstacles arrays may differ from one another in number of obstacles, size of the obstacles, cross sections of the obstacles, dimension of the obstacles, configuration of the array, vertical spacing of the obstacles, and/or direction along which the array is oriented. The plurality of the fluidic channels may be configured to separate different target analytes from a given sample. The plurality of the fluidic channels may be configured to separate a given target analyte from different fluid samples. The plurality of the fluidic channels may be configured to process a plurality of fluid samples simultaneously. The plurality of the fluidic channels may be configured to process at least simultaneously or substantially simultaneously about 2, 3, 4, 5, 6, 7, 8, 9, 10, 12, 14, 16, 18, 20, 25, 30, 35, 40, 45, 50, 60, 70, 80, 90, 100 or more samples.

There may exist a distance between the array of obstacles and a side wall of the microfluidic channel. In some cases, at least one obstacle disposed in the microfluidic channel is adjacent to a side wall of the microfluidic channel. For example, a distance between at least one obstacle disposed in the microfluidic channel and a side wall of the channel may be less than or equal to about 1 μm, 0.5 μm, 0.4 μm, 0.3 μm, 0.2 μm, 0.1 μm, 0.05 μm, or less. The distance between the array of obstacles and a side wall of the microfluidic channel may increase along a direction of fluid flow.

The obstacles (e.g., the array of obstacles) may be configured to separate or isolate one or more target analytes from a fluid flowing through the microfluidic channel. The target analytes may comprise biological particles. The biological particles may be any biological particles described above or elsewhere herein. The target analytes may comprise cells, including any types of cells described above or elsewhere herein. In some cases, the cells comprise senescent cells. In some cases, the cells comprise necrotic cells. The fluid comprising the target analytes may comprise biofluids. The biofluids may be any types of biofluids which may be obtained from a subject. A subject may be any living being comprised of at least one cell. A subject can be a single cell organism or a multi-cellular organism, such as a mammal, a non-mammal (e.g., a bird), or a plant (e.g., a tree). A subject may be a mammal, such as, for example, a human or an animal such as a primate (e.g., a monkey, chimpanzee, etc.), a domesticated animal (e.g., a dog, cat, etc.), farm animal (e.g., goat, sheep, pig, cattle, horse, etc.), or laboratory animal (e.g., mouse, rat, etc.). A subject may be a patient. A subject may be an individual that has or is suspected of having a disease. Examples of subjects may include, but not limited to, humans, mammals, non-human mammals, rodents, amphibians, reptiles, canines, felines, bovines, equines, goats, ovines, hens, avines, mice, rabbits, insects, slugs, microbes, bacteria, parasites, or fish. In some cases, the subject may be a patient who is having, suspected of having, or at a risk of developing a disease or disorder, or encountering an environmental contamination. The biofluids may comprise naturally occurring fluids (e.g., blood, sweat, tears, ear flow, sputum, lymph, bone marrow suspension, urine, saliva, semen, vaginal flow, cerebrospinal fluid, cervical lavage, brain fluid, ascites, milk, secretions of the respiratory, intestinal, or genitourinary tract, amniotic fluid, and water samples), fluids into which cells have been introduced (e.g., culture media and liquefied tissue samples), or combinations thereof. In some cases, the biofluids comprise whole blood. The whole blood may be diluted or undiluted.

The obstacles may be configured to separate or isolate the target analytes using the spaces between the obstacles. The separation or isolation of the target analytes may be based at least partially on sizes or dimensions of the target analytes. It may be desirable that the target analytes have a size or dimension that is greater than or equal to a pre-determined threshold value. The pre-determined threshold value may be identified using reference particles. The reference particles may be directed to flow through a microfluidic channel having obstacles disposed therein. The obstacles may have a known spacing size. Upon flow of the reference particles through the microfluidic channel, a threshold value may be identified. As the separation occurs, the obstacles may be configured to direct the target analytes to flow along a direction that is different from the direction of the fluid flow.

Alternatively, or additionally, the obstacles may be configured to separate or isolate the target analytes using the vertical spacing between the obstacles and a portion or surface of the fluidic channel (e.g., substrate). The vertical spacing for separation or isolation of the target analytes may be based at least partially on sizes or dimensions of the target analytes. The target analytes may have a size or dimension that is greater than or equal to a pre-determined threshold value. Alternatively, the target analytes may have a size or dimension that is less than or equal to a pre-determined threshold value. The pre-determined threshold value may be identified using reference particles. The reference particles may be directed to flow through a microfluidic channel having obstacles disposed therein. The obstacles may have a known vertical gap. Upon flow of the reference particles through the microfluidic channel, a threshold value may be identified. As the separation occurs, the obstacles may be configured to direct the target analytes to flow along a direction that is different from the direction of the fluid flow.

In some cases, at least a subset of the obstacles may have certain flexibility. The obstacles may function as cantilevers, which only have one end fixed. For example, the obstacles may be immobilized on a surface (e.g., channel bottom surface or top surface) of the microfluidic channel and may have a height that is less than or equal to a height (or depth) of the microfluidic channel. Such flexibility of the obstacles may allow for deformation of the obstacles under certain situations, for example, when a flow rate of the fluid comprising the target analytes is greater than a threshold value. In some cases, at least a subset of the obstacles may deform when experiencing a fluidic pressure, which may create shutters in the vertical direction responsive to the fluidic pressure. The shutters may help to release backpressure, thus reducing clogging in the microfluidic channel.

As provided herein, the microfluidic devices may comprise one or more additional components. For example, the microfluidic devices may comprise one or more fluid inlets. The fluid inlets may be in fluidic communication with the fluidic channel. The fluid inlets may be configured to receive fluids and direct the fluids into the microfluidic channel. The fluid inlets may comprise at least a first fluid channel and a second fluid channel. The first fluid channel and the second fluid channel may or may not be in fluidic communication with each other. The first fluid channel may receive a sample fluid comprising one or more target analytes. The second fluid channel may receive an additional fluid from a source. The additional fluid may comprise a sheath fluid. The fluid inlets may each be oriented along a direction that is angled to a length of the fluidic channel with which they are in fluidic communication. The fluid inlets may have a cross sectional dimension that is the same as or different from the fluidic channel.

The microfluidic devices may comprise one or more fluid outlets. The fluid outlets may be in fluidic communication with the fluidic channel. The fluid outlets may each be oriented along a direction that is angled to a length of the fluidic channel with which they are in fluidic communication. The fluid outlets may have a cross sectional dimension that is the same as or different from the fluidic channel. The fluid outlets may comprise a first fluid outlet and a second fluid outlet. The first fluid outlet and the second fluid outlet may or may not be in fluidic communication with each other. The first fluid outlet may receive the target analytes separated from the fluid. The second fluid outlet may receive the remaining fluid (e.g., fluid absent at least a portion of the target analytes). The remaining fluid may flow in the microfluidic channel along the same direction as the original fluid (i.e., the fluid prior to separation). The microfluidic devices may comprise additional fluid outlets (e.g., 3, 4, 5, 6, 7, 8, 9, 10, or more fluid outlets). The additional fluid outlets may receive one or more additional particles (e.g., separated from the fluid) of a particular type or kind (e.g., sharing a certain size or dimension or a certain biological classification or function).

The microfluidic device may comprise a main fluidic channel with one or more inlets and one or more outlets. The one or more outlets may be in fluidic communication with one or more side channels. The one or more side channels may receive a portion of the fluid (e.g., enriched or depleted for a target analyte). In an example, a first side channel may receive a portion of fluid which has passed through an obstacle array as disclosed herein. The portion of fluid is depleted for one or more target analytes which were sorted (e.g., deflected) by the array of obstacles. The second side channel may receive another portion of fluid which is enriched for the one or more target analytes sorted by the array of obstacles. In a further example, the second side channel is itself fluidically connected to two outlets which are in turn connected to further side channels. The second channel has disposed in it a second array of obstacles. The second array of obstacles may be substantially the same as the array of obstacles disposed in the main fluidic channel, or it may differ from the array of obstacles in the main fluidic channel in one or more of vertical spacing, inter-obstacle distance, distance between one or more lines of obstacles, or cross section.

Alternatively or additionally, the microfluidic devices may comprise an additional fluidic component in fluidic communication with the fluidic channel. The additional fluidic component may be configured to receive at least a portion of (e.g., greater than or equal to about 10%, 15%, 20%, 25%, 30%, 35%, 40%, 45%, 50%, 55%, 60%, 65%, 70%, 75%, 80%, 85%, 90%, 95% (mol %), or more) the target analytes separated from the fluid. The additional fluidic component may be used to remove the target analytes from the microfluidic devices. In some cases, the additional fluidic component is a tubing. The tubing may be a microtubing. The additional fluidic component may further be in communication with one or more sample inlets of detection, processing and/or analysis devices. The additional fluidic component may be configured to direct at least a portion of received target analytes into the detection, processing and/or analysis devices for detection, processing and/or analysis. In some cases, the additional fluidic component may be configured to remove large volume or quantity of separated analytes from the microfluidic chips. With the aid of the additional fluidic component, a microfluidic device as provided herein may be capable of processing large quantity of fluid samples or fluid samples having a large volume (a fluid sample having a volume that is greater than or equal to about 10 mL, 15 mL, 20 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, or more). In some cases, a microfluidic device comprises a plurality of additional fluidic components in fluidic communication with the fluidic channel (e.g., a plurality of tubings with the same or different sizes).

In some cases, the additional fluidic component may be configured to recycle a portion of the fluidic stream (e.g., that has been depleted or one or more target analytes) back through the device. Such fluidic components may be in fluidic communication with an outlet and an inlet of the fluidic device. Recycling the depleted fluid back through the device may allow the device or system to operate in a continuous flow manner while separating particles at high efficiency (e.g., at an efficiency greater than about 50%, 55%, 60%, 65%, 70%, 72%, 74%, 76%, 78%, 80%, 82%, 84%, 86%, 88%, 90%, 91%, 92%, 93%, 94%, 95%, 96%, 97%, 98%, 99%, or more).

In some cases, an additional fluidic channel may be comprised in the microfluidic device. The additional fluidic channel may be in fluidic communication with the fluidic channel. The additional fluidic channel may be configured to receive and retain at least a portion of the target analytes. The additional fluidic channel may comprise one or more obstacles disposed therein. The one or more obstacles may be an array of one or more obstacles. The one or more obstacles may or may not be oriented at a single direction. In some cases, the one or more obstacles are uniformed distributed within the additional fluidic channel. The one or more obstacles may be configured to capture the target analytes. The one or more obstacles may have a V-shaped or U-shaped configuration. Each of the one or more obstacles may comprise an opening. The opening may have a dimension that is configured to retain the captured target analytes. The opening may have a size that is greater than or equal to a size the target analytes. The one or more obstacles may be utilized to process samples having a small volume (e.g., a volume that is less than or equal to about 2,000 microliters (μL), 1,500 μL, 1,000 μL, 950 μL, 900 μL, 850 μL, 800 μL, 750 μL, 700 μL, 650 μL, 600 μL, 550 μL, 500 μL, 450 μL, 400 μL, 350 μL, 300 μL, 250 μL, 200 μL, 180 μL, 160 μL, 140 μL, 120 μL, 100 μL, 90 μL, 80 μL, 70 μL, 60 μL, 50 μL, 45 μL, 40 μL, 35 L, 30 μL, 25 μL, 20 μL, 15 μL, 10 μL, 8 μL, 6 μL, 5 μL, 4 μL, 3 μL, 2 μL, 1 μL, or less). The one or more obstacles may facilitate separation and capturing of target analytes from a fluid sample which comprises the target analytes at a low concentration (e.g., target analytes has a concentration less than or equal to about 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05% (vol %, wt%, or mol %), or less). The one or more obstacles may facilitate separation and capturing of target analytes from a fluid sample which comprises a small number of the target analytes (e.g., a fluid which comprise less than or equal to about 50,000, 45,000, 40,000, 35,000, 30,000, 25,000, 20,000, 15,000, 10,000, 9,500, 9,000, 8,500, 8,000, 7,500, 7,000, 6,500, 6,000, 5,500, 5,000, 4,500, 4,000, 3,500, 3,000, 2,500, 2,000, 1,500, 1,000, 900, 800, 700, 600, 500 target analytes, or less).

As will be appreciated, the microfluidic devices may further comprise one or more fluidic pumps. The one or more fluidic pumps may be configured to transport fluidics within the microfluidic devices. The one or more fluidic pumps may be in fluidic communication with the fluidic channel, the fluid inlets, the fluid outlets, the additional fluidic channel, and/or any other components of the microfluidic device. The one or more fluidic pumps may comprise a plurality of valves. The one or more fluidic pumps may comprise a peristaltic pump.

In some aspects, a microfluidic device of the present disclosure may comprise a fluidic channel and one or more obstacles disposed therein. The one or more obstacles may be obstacles as described above or elsewhere herein. The one or more obstacles may be uniformly distributed within the fluidic channel. The one or more obstacles may comprise any number of individual obstacles, for example, greater than or equal to about 2, 3, 4, 5, 6, 7, 8, 9, 10, 11, 12, 13, 14, 15, 16, 17, 18, 19, 20, 22, 24, 26, 28, 30, 35, 40, 45, 50, 55, 60, 65, 70, 75, 80, 85, 90, 100, 110, 120, 130, 140, 150, 160, 170, 180, 190, 200 obstacles or more. The one or more obstacles may be an array of obstacles. The array of obstacles may or may not be oriented or aligned along a direction. The array of obstacles may be oriented at an angle relative to a direction of a fluid flow in the fluidic channel. The angle may be greater than 0°. The angle may be less than 90°. The angle may be any value that is greater than 0° and less than 90°, for example, from about 1° to 85°, or from 5° to 30°. The one or more obstacles may be configured to separate one or more target analytes (e.g., particles) from a fluid flowing through the fluidic channel. The fluid may be any fluids as described above or elsewhere herein. For example, the fluid may comprise biofluids including naturally occurring fluids (e.g., blood, sweat, tears, ear flow, sputum, lymph, bone marrow suspension, urine, saliva, semen, vaginal flow, cerebrospinal fluid, cervical lavage, brain fluid, ascites, milk, secretions of the respiratory, intestinal, or genitourinary tract, amniotic fluid, and water samples), fluids into which cells have been introduced (e.g., culture media and liquefied tissue samples), or combinations thereof.

The target analytes may be any analytes that are of interest. The target analytes may be particles, such as biological particles as described above or elsewhere herein. For example, the target analytes may comprise any cells or components thereof, viruses, bacteria, proteins, carbohydrates, nucleic acid molecules (such as deoxyribonucleic acid (DNA), ribonucleic acid (RNA)), lipid, or combinations thereof. Non-limiting examples of cells may include, tumor cells, red blood cells, white blood cells (such as T cells, B cells, and helper T cells), infected cells, trophoblasts, fibroblasts, stem cells, epithelial cells, infectious organisms (e.g., bacteria, protozoa, and fungi), cancer cells, bone marrow cells, fetal cells, progenitor cells, foam cells, mesenchymal cells, immune system cells, endothelial cells, endometrial cells, connective tissue cells, trophoblasts, bacteria, fungi, or pathogens, or combinations thereof.

In some cases, cells may comprise senescent cells. The senescent cells may comprise senescent tumor cells. Senescent tumor cells may comprise tumor cells that are benign or malignant. Non-limiting examples of tumors may include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastrointestinal system carcinomas, colon carcinoma, pancreatic cancer, breast cancer, genitourinary system carcinomas, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, endocrine system carcinomas, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, or combinations thereof. The tumors may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof. The obstacles (e.g., an array of obstacles) may separate one or more senescent cells from a fluid upon flow of the fluid through the microfluidic channel. The fluid may comprise one or more non-senescent cells, in addition to the senescent cells. The obstacles may separate the one or more senescent cells from the non-senescent cells while the fluid flows through the fluidic channel. The obstacles may separate the senescent cells at a high efficiency (e.g., at an efficiency greater than about 50%, 55%, 60%, 65%, 70%, 72%, 74%, 76%, 78%, 80%, 82%, 84%, 86%, 88%, 90%, 91%, 92%, 93%, 94%, 95%, 96%, 97%, 98%, 99%, or more).

In some cases, cells may comprise necrotic cells. The cells may comprise necrotic tumor cells. The cells may comprise necrotic stem cells. The obstacles (e.g., an array of obstacles) may separate one or more senescent cells from a fluid upon flow of the fluid through the microfluidic channel. The fluid may comprise one or more non-necrotic cells, in addition to the necrotic cells. The obstacles may separate the one or more necrotic cells from the non-necrotic cells while the fluid flows through the fluidic channel. The obstacles may separate the necrotic cells at a high efficiency (e.g., at an efficiency greater than about 50%, 55%, 60%, 65%, 70%, 72%, 74%, 76%, 78%, 80%, 82%, 84%, 86%, 88%, 90%, 91%, 92%, 93%, 94%, 95%, 96%, 97%, 98%, 99%, or more).

The target analytes can be of any size, shape, or geometry. The target analytes may have an average size that is greater than or equal to about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, or more. In some cases, the target analytes have an average size that falls between any of the two values described above or elsewhere herein, for example, from about 15 μm to 30 μm.

The one or more obstacles may be configured to separate one or more target analytes with a high throughput. The microfluidic device of the present disclosure may be configured to process a fluid sample having a volume that is greater than or equal to about 10 mL, 15 mL, 20 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, or more. In cases where a large quantity of sample is to be processed or multiplex assaying is desired, the system of the present disclosure may comprise a plurality of microfluidic devices, e.g., greater than or equal to about 2, 3, 4, 5, 6, 7, 8, 9, 10, 11, 12, 13, 14, 15, 16, 17, 18, 19, 20, 25, 30 devices or more. The plurality of devices may or may not be in fluidic communication with one another. The plurality of devices may be in fluidic communication with one or more common fluid inlets and/or outlets. The plurality of devices may be arranged in parallel, in series or in a combined configuration of in series and in parallel. In some examples, individual devices of the plurality of devices may be stacked in vertical direction (or a direction perpendicular to a plane within which the fluidic channel is disposed. Individual devices of the plurality of devices may or may not be the same in terms of size, shape, geometry, sample processing capability, and/or obstacles (e.g., number of obstacles, shape, size, dimension, geometry, arrangement of the obstacles) comprised in the fluidic channel.

In some examples, a single microfluidic device may comprise multiple fluidic channels (e.g., greater than or equal to about 2, 3, 4, 5, 6, 7, 8, 9, 10, 12, 14, 16, 18, 20, 25, 30, 35, 40, 45, 50 fluidic channels or more). The fluidic channels may or may not be in fluidic communication with one another. The fluidic channels may be arranged in parallel, in series or in combined configuration of in parallel and in series. The fluidic channels may each comprise one or more obstacles (e.g., an array of obstacles). Obstacles comprised in different fluidic channels may be the same or may be different. Obstacle arrays may differ from one another in number of obstacles comprised in the array, size, dimension, shape, geometry, cross sections, configuration of obstacles, spacing size between adjacent obstacles of the array, vertical spacing, and/or arrangement of obstacles in the array. The fluidic channels may be configured to process the same sample. The fluidic channels may each be configured to process a different sample. The fluidic channels may each be configured to separate a different type of target analytes from a fluid sample. It should be noted that the disclosure is not limited to the various examples described above and elsewhere herein. For example, in some cases, instead of having multiple microfluidic devices or a single device having multiple channels, various types of target analytes may be separated from a fluid using a microfluidic device comprising a fluidic channel which comprises multiple sections along a direction of fluid flow. Each section of the fluidic channel may comprise a different array of obstacles which is configured to separate, isolate and/or capture a given type of analytes.

The target analytes may be separated with a high efficiency when the fluid is directed to flow through the fluidic channel at a given flow rate. As provided herein, the flow rate may be greater than or equal to about 10 milliliters/hour (mL/hr), 20 mL/hr, 40 mL/hr, 60 mL/hr, 80 mL/hr, 100 mL/hr, 120 mL/hr, 140 mL/hr, 160 mL/hr, 180 mL/hr, 200 mL/hr, 220 mL/hr, 240 mL/hr, 260 mL/hr, 280 mL/hr, 300 mL/hr, 320 mL/hr, 340 mL/hr, 360 mL/hr, 380 mL/hr, 400 mL/hr, 420 mL/hr, 440 mL/hr, 460 mL/hr, 480 mL/hr, 500 mL/hr, 550 mL/hr, 600 mL/hr, 650 mL/hr, 700 mL/hr, 750 mL/hr, 800 mL/hr, 850 mL/hr, 900 mL/hr, 950 mL/hr, 1,000 mL/hr, or more. In some cases, the flow rate is between any of the two values described above and elsewhere herein, for example, about 250 mL/hr.

The separation efficiency may be determined as a percentage (e.g., number or mole percent) of original target analytes comprised in the fluid that is separated from the fluid by the obstacles. For example, upon flow of a fluid comprising 10,000 particles through the fluidic channel, if 5,000 particles are separated or isolated from the fluid, then the efficiency is 50%. In another example, if 70 mol % of the target analytes that are originally comprised in a fluid is separated from the fluid as the fluid flows through the fluidic channel, then the efficiency is 70%. As provided herein, the target analytes may be separated from the fluid with a high efficiency. The efficiency may be greater than or equal to about 50%, 55%, 60%, 65%, 70%, 72%, 74%, 76%, 78%, 80%, 82%, 84%, 86%, 88%, 90%, 91%, 92%, 93%, 94%, 95%, 96%, 97%, 98%, 99%, or more. In some cases, the target analytes are separated from the fluid at an efficiency that falls between any of the two values described above or elsewhere herein, for example, about 75%.

In some aspects, the systems of the present disclosure comprise a microfluidic device which may separate target analytes from small sample volumes. The microfluidic device may comprise a fluidic channel. The fluidic channel may comprise one or more obstacles disposed therein. The obstacles may be any obstacles as described above or elsewhere herein. The obstacles may comprise microstructures, nanostructures, or combinations thereof. At least a subset of the obstacles is nonporous. In some cases, all of the obstacles are nonporous. The obstacles may be 3D structures. The obstacles may have openings in x-, y-and z-directions. The obstacles may deform when experiencing a pressure. An average spacing size between adjacent obstacles may vary. The average spacing size may be adjusted depending upon a variety of factors, including such as dimension of the microfluidic channel, number of obstacles disposed in the microfluidic channel, sample volume, sizes, dimensions, geometries of target analytes, fluid flow rate, or combinations thereof. In some cases, the obstacles may have an average spacing size greater than or equal to about 10 nanometers (nm), 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 5 μm, 10 μm, 20 μm, 30 μm, 40 μm, 50 μm, 60 μm, 70 μm, 80 μm, 90 μm, 100 μm, or more. In some cases, the average spacing size may be less than or equal to about 200 μm, 180 μm, 160 μm, 140 μm, 120 μm, 100 μm, 85 μm, 75 μm, 65 μm, 55 μm, 45 μm, 35 μm, 25 μm, 15 μm, 5 μm, 1 μm, 850 nm, 750 nm, 650 nm, 550 nm, 450 nm, 350 nm, 250 nm, 150 nm, 100 nm, or less. In some cases, the average spacing size may be any of the values described above or elsewhere herein, for example, from about 1 μm to 100 μm.

Surfaces of the obstacles may be modified. For example, the obstacles may be coated with chemical or biological reagents, e.g., a charged moiety, an antibody. The obstacles may be treated with reagents such that they may bind specifically to a given type of target analytes. Non-limiting examples of reagents that may be used for treating, modifying the obstacles include polymers, carbohydrates, a molecule that binds to a cell surface receptor, an oligo-or polypeptide, a viral or bacterial protein, a nucleic acid, or a carbohydrate that binds a population of cells, or combinations thereof.

The one or more obstacles may comprise an array of obstacles. The obstacles may be configured to separate one or more target analytes from a fluid having a small volume upon flow of the fluid through the fluidic channel. The target analytes may comprise any analytes as described above or elsewhere herein, for example, biological particles. In some cases, the target analytes comprise senescent cells.

The fluid may have a volume that is less than or equal to about 2,000 microliters (μL), 1,500 μL, 1,000 μL, 950 μL, 900 μL, 850 μL, 800 μL, 750 μL, 700 μL, 650 μL, 600 μL, 550 μL, 500 μL, 450 μL, 400 μL, 350 μL, 300 μL, 250 μL, 200 μL, 180 μL, 160 μL, 140 μL, 120 μL, 100 μL, 90 μL, 80 μL, 70 μL, 60 μL, 50 μL, 45 μL, 40 μL, 35 μL, 30 μL, 25 μL, 20 μL, 15 μL, 10 μL, 8 μL, 6 μL, 5 μL, 4 μL, 3 μL, 2 μL, 1 μL, or less. In some cases, the fluid has a volume that is between any of the two values described above and elsewhere herein, for example, from about 1 μL to 500 μL.

The fluid may comprise a small number of target analytes. For example, the fluid may comprise less than or equal to about 50,000, 45,000, 40,000, 35,000, 30,000, 25,000, 20,000, 15,000, 10,000, 9,500, 9,000, 8,500, 8,000, 7,500, 7,000, 6,500, 6,000, 5,500, 5,000, 4,500, 4,000, 3,500, 3,000, 2,500, 2,000, 1,500, 1,000, 900, 800, 700, 600, 500 target analytes, or less. In some cases, the number of target analytes comprised in the fluid may be between any of the two values describe above or elsewhere herein, for example, from about 1,000 to about 20,000.

In some cases, the fluid may comprise target analytes at a low concentration. For example, the fluid may comprise the target analytes at a concentration less than or equal to about 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05% (vol %, wt%, or mol %), or less. In some cases, the target analytes have a concentration between any of two values describe above or elsewhere herein, for example, from about 1% to about 10%.

As provided herein, the microfluidic devices may be monolithic, or may be fabricated in one or more components which may be assembled. Various components or layers of the devices may be assembled or bonded together using various methods or tools including, e.g., adhesives, clamps, heat, anodic heating, or reactions.

### Methods

Also provided herein are methods for separating, isolating, detecting, and/or analyzing target analytes such as biological particles. Such methods may be used for processing senescence cells. In some cases, the methods may be used for processing necrotic cells. In some cases, the methods may be used for processing mixtures of live, senescent, and necrotic cells.

In an aspect, a method may comprise directing a fluid comprising one or more target analytes into a microfluidic device. The microfluidic device may be any microfluidic devices described above or elsewhere herein. For example, the microfluidic device may comprise a fluidic channel and one or more obstacles disposed therein. The obstacles may be any obstacles of the present disclosure. The one or more obstacles may be an array of obstacles. The array of obstacles may be oriented or aligned along a certain direction. The array of obstacles may comprise a vertical spacing. The direction along which the obstacle array is aligned may be angled relative to a direction of fluid flow in said fluidic channel. The direction of fluid flow may be a direction along which the fluid comprising the target analytes flows within the fluidic channel. The direction of fluid flow may not change as the fluid flows through the fluidic channel. There may be an angle between the direction along which the array of obstacles is aligned and the direction of the fluid flow. The angle may be an oblique angle. The angle may be from about 0° to about 90°. In some cases, the angle may be greater than about 0°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85° or more. In some cases, the angle may less than about 90°, 85°, 80°, 75°, 70°, 65°, 60°, 55°, 50°, 45°, 40°, 35°, 30°, 25°, 20°, 15°, 10°, 9°, 7°, 5°, 3°, 1°, or less. In some cases, the angle may be between any of the values described above or elsewhere herein, for example, from about 0° to 20°. In some cases, all of the obstacles are angled relative to the direction of the fluid flow.

The microfluidic device may comprise a substrate with a fluidic channel disposed in or along the substrate. The fluidic channel may comprise a top surface defining a ceiling of the fluidic channel and opposing a surface of the substrate. The fluidic channel may comprise a main channel comprising an inlet and an outlet. The main channel may comprise a length defining an x-axis direction, a width defining a y-axis direction, and a height defining a z-axis direction. The fluidic channel may further comprise a first side channel connected to the outlet of the main channel. The fluidic channel may additionally comprise a second side channel connected to the outlet of the main channel. The main channel may comprise an array of obstacles disposed therein. The obstacles may extend from the ceiling of the channel toward the surface of the substate, substantially along the z-axis direction. The obstacles may comprise a length which is shorter than the height of the main channel, thereby separating the obstacles from the surface of the substate by the vertical spacing.

In some cases, the array of obstacles comprises a first line of obstacles and a second line of obstacles. The first line of obstacles may be separated from the second line of obstacles by a distance along the y-axis. The average distance along the y-axis may be about 10 nanometers (nm), 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 5 μm, 10 μm, 20 μm, 30 μm, 40 μm, 50 μm, 60 μm, 70 μm, 80 μm, 90 μm, 100 μm, or more. In some cases, the average distance may be less than or equal to about 200 μm, 180 μm, 160 μm, 140 μm, 120 μm, 100 μm, 85 μm, 75 μm, 65 μm, 55 μm, 45 μm, 35 μm, 25 μm, 15 μm, 5 μm, 1 μm, 850 nm, 750 nm, 650 nm, 550 nm, 450 nm, 350 nm, 250 nm, 150 nm, 100 nm, or less. The line of obstacles may be oriented at an angle relative to the x-axis, such as from 1° to 90°. In some cases, a line of obstacles comprises obstacles comprising a substantially the same parallelogram cross section. The substantially the same parallel cross section may comprise an acute angle. The acute angle may be less than the angle of the line of obstacles. In an example, the angle of orientation of the obstacles comprises from about 1° to about 85° and the angle of the parallelogram cross section comprises from about 2° to about 89°. In another example, the angle of orientation of the obstacles comprises from about 3° to about 30° and the angle of the parallelogram cross section comprises from about 5° to about 50°. The obstacles of the line of obstacles may be separated by an inter-obstacle spacing. The inter-obstacle spacing may be in a plane defined by the x-axis and the y-axis. The inter-obstacle spacing may comprise a shortest or longest distance between two opposing faces of two obstacles in the line. The inter-obstacle spacing may comprise a distance such that analytes (e.g., biological particles) less than a threshold value pass through the inter-obstacle spacing while analytes greater than or equal to the threshold roll along the obstacles. The analytes above the threshold may be deflected by the rolling. In some cases, the inter-obstacle distance is at least about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm.

Next, the fluid comprising the target analytes is directed to flow through the fluid channel. Upon flow of the fluid through the fluidic channel, at least a portion of the target analytes may be separated from the fluid with the aid of the obstacles. The obstacles may be configured to direct some or all of the target analytes that are separated from the fluid to flow along or towards a direction which differs from the direction of fluid flow. As described above or elsewhere herein, the obstacles may separate the target analytes based at least partially on sizes of the target analytes. The obstacles may have an average spacing size (e.g., inter-obstacle distance) which may permit analytes having an average size below a threshold value to pass through while hindering the movement of analytes having an average size equal to or above the threshold value (e.g., causing them to roll along the obstacles). The threshold value may or may not be an average spacing size of the obstacles. The threshold value may be determined using reference analytes (e.g., reference particles having known sizes). In some cases, the average spacing size may be adjusted for separating different types of target analytes. The adjustment may be achieved by removing, adding and/or substituting one or more obstacles disposed in the fluidic channel. For example, one or more obstacles may be removed from the fluidic channel to increase an average spacing size of the obstacles. Similarly, in cases where a smaller average spacing size is desired, one or more obstacles may be added to the fluidic channel. In some cases, the average spacing size may be altered by substituting one or more obstacles with different types of obstacles, e.g., obstacles with different cross sections, dimensions, geometries etc.

Alternatively or additionally, the obstacles may have an average vertical spacing which may permit analytes having an average size below a threshold value to pass through while hindering the movement of analytes having an average size equal to or above the threshold value. The vertical spacing may be adjusted for separating different types of analytes.

As described above and elsewhere herein, a distance may exist between the array of obstacles and a side wall of the microfluidic channel. In some cases, at least one obstacle disposed in the microfluidic channel is adjacent to a side wall of the microfluidic channel. For example, a distance between at least one obstacle disposed in the microfluidic channel and a side wall of the channel may be less than or equal to about 1 μm, 0.5 μm, 0.4 μm, 0.3 μm, 0.2 μm, 0.1 μm, 0.05 μm, or less. The distance between the array of obstacles and a side wall of the microfluidic channel may increase along a direction of fluid flow.

The target analytes may be any types of target analytes as described above or elsewhere herein. For example, the target analytes may be biological particles. For example, the target analytes may comprise any cells or components thereof, viruses, bacteria, proteins, carbohydrates, nucleic acid molecules (such as deoxyribonucleic acid (DNA), ribonucleic acid (RNA)), lipid, or combinations thereof. Non-limiting examples of cells may include, tumor cells, red blood cells, white blood cells (such as T cells, B cells, and helper T cells), infected cells, trophoblasts, fibroblasts, stem cells, epithelial cells, infectious organisms (e.g., bacteria, protozoa, and fungi), cancer cells, bone marrow cells, fetal cells, progenitor cells, foam cells, mesenchymal cells, immune system cells, endothelial cells, endometrial cells, connective tissue cells, trophoblasts, bacteria, fungi, or pathogens, or combinations thereof. In some cases, cells may comprise senescent cells. The senescent cells may comprise senescent tumor cells. The senescent cells may comprise stem cells. In some cases, cells may comprise necrotic cells. The necrotic cells may comprise necrotic stem cells.

In some cases, the method further comprises directing an additional fluid into the microfluidic device. The additional fluid may or may not the same as the fluid that comprises the target analytes. The additional fluid and the fluid may be miscible, partially miscible, or immiscible. The additional may comprise a sheath fluid, e.g., a buffer. The additional fluid may be used to ensure that the fluid is flowing along or towards a certain direction (e.g., the direction of fluid flow).

While the target analytes are separated from the fluid, at least a portion (e.g., greater than or equal to about 20%, 25%, 30%, 35%, 40%, 45%, 50%, 55%, 60%, 65%, 70%, 75%, 80%, 85%, 90%, 95%, or more) of the separated target analytes may be captured. The target analytes may be captured by one or more obstacles disposed in a fluidic component (e.g., an additional fluid channel) comprised in the microfluidic device. The additional fluidic channel may be in fluidic communication with the fluidic channel. The one or more obstacles may or may not be the same as the obstacles disposed in the fluidic channel. The one or more obstacles may be an array of obstacles. The one or more obstacles may be randomly or uniformly distributed in the additional fluidic channel. The one or more obstacles may have a V-shaped pattern. Each of the one or more obstacles may comprise an opening. The opening may have a dimension that is configured to retain the captured target analytes. The opening may have a size that is greater than or equal to a size the target analytes.

In some cases, the separated target analytes may be detected. In some cases, the separated target analytes may be removed without any further analyses. In some cases, the separated target analytes may be directed to one or more detection and/or analysis units for detection and/or analyses.

As provided herein, the methods of the present disclosure may separate or isolate target analytes from a fluid at a high sensitivity. The sensitivity may be determined as a ratio of (i) target analytes separated from the fluid to (ii) a total of target analytes and non-target analytes separated from the fluid. For example, if 50% of the analytes separated from the fluid are target analytes, then the sensitivity is 50%. The methods of the present disclosure may separate or isolate target analytes at a sensitivity greater than or equal to about 60%, 65%, 70%, 75%, 80%, 82%, 84%, 86%, 88%, 90%, 91%, 92%, 93%, 94%, 95%, 96%, 97%, 98%, 99%, or more.

The methods of the present disclosure may separate or isolate target analytes from a fluid at a high specificity. The specificity may be determined as a ratio of (i) non-target analytes remained in (or not separated from) the fluid to (ii) a total of target analytes and non-target analytes remained in (or not separated from) the fluid. As an example, if 50% of the analytes remained in the fluid are non-target analytes, then the specificity is 50%. The methods of the present disclosure may separate or isolate target analytes at a specificity greater than or equal to about 60%, 65%, 70%, 75%, 80%, 82%, 84%, 86%, 88%, 90%, 91%, 92%, 93%, 94%, 95%, 96%, 97%, 98%, 99%, or more.

In some aspects of the present disclosure, the methods may comprise directing a fluid comprising one or more target analytes into a microfluidic device. The microfluidic device may be any microfluidic devices described above or elsewhere herein. For example, the microfluidic device may comprise a fluidic channel and one or more obstacles disposed therein. The one or more obstacles may be obstacles as described above or elsewhere herein. The one or more obstacles may be comprised in an array of obstacles. The array of obstacles may or may not be oriented or aligned along a single direction. The one or more obstacles may be configured to separate one or more target analytes (e.g., particles) from a fluid flowing through the fluidic channel. The one or more obstacles may comprise a vertical spacing as described herein. The vertical spacing may be configured to separate a target analyte of the one or more target analytes from the fluid. The fluid may be any fluids as described above or elsewhere herein. For example, the fluid may comprise biofluids. The biofluid may comprise whole blood, plasma, serum, or a portion or fraction thereof. The whole blood may be diluted or undiluted.

Next, the fluid may be directed to flow through the fluidic channel. Upon flow of the fluid through the fluidic channel, at least a portion of the target analytes may be separated from the fluid using the one or more obstacles. The methods may separate the target analytes by deflecting at least a portion of the one or more particles while allowing another portion of the one or more analytes to pass through the vertical spacing or inter-obstacle spacing of the one or more obstacles. The vertical spacing may be determined such that analytes having a characteristic dimension (e.g., diameter) less than a cutoff value are able to pass through the vertical spacing or inter-obstacle distance while other analytes having a corresponding dimension greater than or equal to the cutoff value are unable to pass through the vertical spacing and are deflected (e.g., induced to roll along a surface of the obstacles). Analytes which are deflected may continue down a different path or portion of the fluidic device form the remainder of the fluid (e.g., for separation or downstream analysis or processing).

In some cases, the fluidic channel may comprise one or more additional arrays of obstacles. The one or more additional arrays of obstacles may each be characterized by another vertical spacing, such that the additional arrays of obstacles are configured to further separate out additional types (e.g., comprising another corresponding dimension greater or less than a cutoff value) of analytes. Additionally or alternatively, the one or more additional arrays of obstacles may be characterized by another inter-obstacle spacing. In other cases, the vertical spacing or inter-obstacle distance may be the same for each array.

In some cases, the methods may further comprising recycling at least a portion of the fluid to be directed through the fluidic channel one or more additional times. The at least a portion of the fluid may comprise fluid which has passed through the fluidic channel at least once and has been depleted of one or more target analytes. Upon subsequent passes through the fluidic channel, additional analytes may be separated, allowing for a high efficiency of separation (e.g., a separation of at least 50%, 60%, 70%, 80%, 81%, 82%, 83%, 84%, 85%, 86%, 78%, 88%, 89%, 90%, 91%, 92%, 93%, 94%, 95%, 96%, 97%, 98%, 99%, or more)

In some aspects of the present disclosure, the methods may comprise directing a fluid comprising one or more target analytes into a microfluidic device. The microfluidic device may be any microfluidic devices described above or elsewhere herein. For example, the microfluidic device may comprise a fluidic channel and one or more obstacles disposed therein. The one or more obstacles may be obstacles as described above or elsewhere herein. The one or more obstacles may be uniformly distributed within the fluidic channel. The one or more obstacles may comprise any number of individual obstacles, for example, greater than or equal to about 2, 3, 4, 5, 6, 7, 8, 9, 10, 11, 12, 13, 14, 15, 16, 17, 18, 19, 20, 22, 24, 26, 28, 30, 35, 40, 45, 50, 55, 60, 65, 70, 75, 80, 85, 90, 100, 110, 120, 130, 140, 150, 160, 170, 180, 190, 200 obstacles or more. The one or more obstacles may be an array of obstacles. The array of obstacles may or may not be oriented or aligned along a single direction. The one or more obstacles may be configured to separate one or more target analytes (e.g., particles) from a fluid flowing through the fluidic channel. The fluid may be any fluids as described above or elsewhere herein. For example, the fluid may comprise biofluids.

Next, the fluid may be directed to flow through the fluidic channel. Upon flow of the fluid through the fluidic channel, at least a portion of the target analytes may be separated from the fluid using the one or more obstacles. The methods may separate the target analytes with a high efficiency while the fluid is directed to flow through the fluidic channel at a given flow rate. For example, the fluid may be directed through the fluidic channel at a flow rate greater than or equal to about 100 milliliters/hour (mL/hr), 120 mL/hr, 140 mL/hr, 160 mL/hr, 180 mL/hr, 200 mL/hr, 220 mL/hr, 240 mL/hr, 260 mL/hr, 280 mL/hr, 300 mL/hr, 320 mL/hr, 340 mL/hr, 360 mL/hr, 380 mL/hr, 400 mL/hr, 420 mL/hr, 440 mL/hr, 460 mL/hr, 480 mL/hr, 500 mL/hr, 550 mL/hr, 600 mL/hr, 650 mL/hr, 700 mL/hr, 750 mL/hr, 800 mL/hr, 850 mL/hr, 900 mL/hr, 950 mL/hr, 1,000 mL/hr, or more. In some cases, the flow rate is between any of the two values described above and elsewhere herein, for example, about 250 mL/hr.

The separation efficiency may be determined as a percentage (e.g., number or mole percent) of original target analytes comprised in the fluid that is separated from the fluid by the obstacles. For example, upon flow of a fluid comprising 10,000 particles through the fluidic channel, if 5,000 particles are separated or isolated from the fluid, then the efficiency is 50%. In another example, if 70 mol % of the target analytes that are originally comprised in a fluid is separated from the fluid as the fluid flows through the fluidic channel, then the efficiency is 70%. As provided herein, the target analytes may be separated from the fluid with an efficiency greater than or equal to about 50%, 55%, 60%, 65%, 70%, 72%, 74%, 76%, 78%, 80%, 82%, 84%, 86%, 88%, 90%, 91%, 92%, 93%, 94%, 95%, 96%, 97%, 98%, 99%, or more. In some cases, the target analytes are separated from the fluid at an efficiency that falls between any of the two values described above or elsewhere herein, for example, about 75%.

Some aspects of the present disclosure provide a method for separating one or more target analytes from a fluid sample having a small volume. The method may comprise directing a fluid having a small volume into a microfluidic device. The microfluidic device may be any microfluidic devices as described above or elsewhere herein. For example, the microfluidic device may comprise a fluidic channel which may comprise one or more obstacles disposed therein. The obstacles may be any obstacles described above or elsewhere herein. In some examples, the obstacles may be an array of obstacles.

The fluid may have a volume that is less than or equal to about 2,000 microliters (μL), 1,500 μL, 1,000 μL, 950 μL, 900 μL, 850 μL, 800 μL, 750 μL, 700 μL, 650 μL, 600 μL, 550 μL, 500 μL, 450 μL, 400 μL, 350 μL, 300 μL, 250 μL, 200 μL, 180 μL, 160 μL, 140 μL, 120 μL, 100 μL, 90 μL, 80 μL, 70 μL, 60 μL, 50 μL, 45 μL, 40 μL, 35 μL, 30 μL, 25 μL, 20 μL, 15 μL, 10 μL, 8 μL, 6 μL, 5 μL, 4 μL, 3 μL, 2 μL, 1 μL, or less. In some cases, the fluid has a volume that is between any of the two values described above and elsewhere herein, for example, from about 1 μL to 500 μL.

The fluid may comprise a small number of target analytes. For example, the fluid may comprise less than or equal to about 50,000, 45,000, 40,000, 35,000, 30,000, 25,000, 20,000, 15,000, 10,000, 9,500, 9,000, 8,500, 8,000, 7,500, 7,000, 6,500, 6,000, 5,500, 5,000, 4,500, 4,000, 3,500, 3,000, 2,500, 2,000, 1,500, 1,000, 900, 800, 700, 600, 500 target analytes, or less. In some cases, the number of target analytes comprised in the fluid may be between any of the two values describe above or elsewhere herein, for example, from about 1,000 to about 20,000.

In some cases, the fluid may comprise target analytes at a low concentration. For example, the fluid may comprise the target analytes at a concentration less than or equal to about 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05% (vol %, wt%, or mol %), or less. In some cases, the target analytes have a concentration between any of two values describe above or elsewhere herein, for example, from about 1% to about 10%.

As provided above or elsewhere herein, the target analytes may be any analytes that are of interest. The target analytes may be particles, such as biological particles as described above or elsewhere herein. In some examples, the target analytes may comprise any cells or components thereof, viruses, bacteria, proteins, carbohydrates, nucleic acid molecules (such as deoxyribonucleic acid (DNA), ribonucleic acid (RNA)), lipid, or combinations thereof. Non-limiting examples of cells may include, tumor cells, red blood cells, white blood cells (such as T cells, B cells, and helper T cells), infected cells, trophoblasts, fibroblasts, stem cells, epithelial cells, infectious organisms (e.g., bacteria, protozoa, and fungi), cancer cells, bone marrow cells, fetal cells, progenitor cells, foam cells, mesenchymal cells, immune system cells, endothelial cells, endometrial cells, connective tissue cells, trophoblasts, bacteria, fungi, or pathogens, or combinations thereof.

In some cases, cells may comprise senescent cells. The senescent cells may comprise senescent tumor cells. Senescent tumor cells may comprise tumor cells that are benign or malignant. Non-limiting examples of tumors may include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastrointestinal system carcinomas, colon carcinoma, pancreatic cancer, breast cancer, genitourinary system carcinomas, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, endocrine system carcinomas, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, or combinations thereof. The tumors may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof. In some cases, the target analytes may comprise senescent T cells, senescent cells of different kinds of white blood cells, senescent microphages, senescent lung, breast, colon, prostate, gastric, hepatic, ovarian, esophageal, or bronchial epithelial or stromal cells, senescent skin epithelial or stromal cells, senescent glial cells, senescent vascular endothelial or stromal cells, or combinations thereof. The obstacles (e.g., an array of obstacles) may separate one or more senescent cells from a fluid upon flow of the fluid through the microfluidic channel. The fluid may comprise one or more non-senescent cells, in addition to the senescent cells. The obstacles may separate the one or more senescent cells from the non-senescent cells while the fluid flows through the fluidic channel. The obstacles may separate the senescent cells at a high efficiency (e.g., at an efficiency greater than about 50%, 55%, 60%, 65%, 70%, 72%, 74%, 76%, 78%, 80%, 82%, 84%, 86%, 88%, 90%, 91%, 92%, 93%, 94%, 95%, 96%, 97%, 98%, 99%, or more).

In some cases, cells can comprise necrotic cells. The necrotic cells may comprise necrotic tumor cells. The necrotic cells may comprise necrotic stem cells. The obstacles (e.g., an array of obstacles) may separate one or more necrotic cells from a fluid upon flow of the fluid through the microfluidic channel. The fluid may comprise one or more non-necrotic cells (e.g., senescent cells, live cells), in addition to the necrotic cells. The obstacles may separate the one or more senescent cells from the non-senescent cells while the fluid flows through the fluidic channel. The obstacles may separate the senescent cells at a high efficiency (e.g., at an efficiency greater than about 50%, 55%, 60%, 65%, 70%, 72%, 74%, 76%, 78%, 80%, 82%, 84%, 86%, 88%, 90%, 91%, 92%, 93%, 94%, 95%, 96%, 97%, 98%, 99%, or more).

The target analytes can be of any size, shape, or geometry. The target analytes may have an average size that is greater than or equal to about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, or more. The target analytes may have an average size that is less than or equal to about 50 μm, 45 μm, 40 μm, 35 μm, 30 μm, 28 μm, 26 μm, 24 μm, 22 μm, 20 μm, 18 μm, 16 μm, 14 μm, 12 μm, 10 μm, 9 μm, 8 μm, 7 μm, 6 μm, 5 μm, 4 μm, 3 μm, 1 μm, 1 μm, or less. In some cases, the target analytes have an average size that falls between any of the two values described above or elsewhere herein, for example, from about 15 μm to 30 μm.

The method may further comprise directing the fluid to flow through the fluidic channel. Upon flow of the fluid through the fluidic channel, at least a portion of the target analytes may be separated or removed from the fluid using the obstacles. The method may separate at least about 60%, 65%, 70%, 75%, 80%, 85%, 90%, 95% of the target analytes, or more. The method may separate from the fluid less than or equal to about 30%, 25%, 20%, 18%, 16%, 14%, 12%, 10%, 8%, 6%, 4%, 2%, 1% of non-target analytes. In some cases, the obstacles may separate the target analytes by causing the target analytes to flow at or towards a direction which is different from a direction of the fluid in the fluidic channel. The direction of the fluid before and after removal of the target analytes may remain unchanged. During or after the separation, the target analytes and the fluid having at least a portion of the target analytes removed therefrom may flow out of the fluidic channel along different directions. For example, the separated target analytes and the fluid having at least a portion of the target analytes removed therefrom may be directed to a first fluid outlet and a second fluid outlet, respectively.

The target analytes separated from the fluid may be captured. The target analytes may be captured using one or more obstacles disposed in a fluidic component (e.g., an additional fluidic channel) of the microfluidic device. The one or more obstacles may or may not be the same as the obstacles disposed in the fluidic channel. The one or more obstacles used to capture the target analytes may be an array of capture obstacles. The one or more obstacles may be randomly or uniformly distributed in the additional fluidic channel. The one or more obstacles may have a V-shaped, or U-shaped pattern. Each of the one or more obstacles may comprise an opening. The opening may have a dimension that is configured to retain the captured target analytes. The opening may have a size that is greater than or equal to a size the target analytes.

The method may separate or isolate target analytes from a fluid at a high sensitivity. The sensitivity may be determined as a ratio of (i) target analytes separated from the fluid to (ii) a total of target analytes and non-target analytes separated from the fluid. For example, if 50% of the analytes separated from the fluid are target analytes, then the sensitivity is 50%. The methods of the present disclosure may separate or isolate target analytes at a sensitivity greater than or equal to about 60%, 65%, 70%, 75%, 80%, 82%, 84%, 86%, 88%, 90%, 91%, 92%, 93%, 94%, 95%, 96%, 97%, 98%, 99%, or more.

The methods of the present disclosure may separate or isolate target analytes from a fluid at a high specificity. The specificity may be determined as a ratio of (i) non-target analytes remained in (or not separated from) the fluid to (ii) a total of target analytes and non-target analytes remained in (or not separated from) the fluid. As an example, if 50% of the analytes remained in the fluid are non-target analytes, then the specificity is 50%. The methods of the present disclosure may separate or isolate target analytes at a specificity greater than or equal to about 60%, 65%, 70%, 75%, 80%, 82%, 84%, 86%, 88%, 90%, 91%, 92%, 93%, 94%, 95%, 96%, 97%, 98%, 99%, or more.

The target analytes may be detected during and/or after separation of the target analytes from the fluid. The detection may be performed in real-time while the separation is taking place. The detection may be performed at multiple time points while the separation is taking place. The detection may be performed subsequent to separation of the target analytes from the fluid. The detection may be performed on the microfluidic device. The detection may be performed after removing the target analytes from the microfluidic device. The detection may comprise detecting a presence or absence of the target analytes. The detection may comprise detecting an amount of the target analytes. The detection may comprise detecting a signal from the target analytes. The signal may be an optical signal. The optical signal may be an optical signal of any wavelength or frequency. The optical signal may comprise visible light, ultraviolet light and/or infrared light. The optical signal may be luminescent signals (e.g., bioluminescence, chemiluminescence, fluorescence). The signal may be an electrical signal. The electrical signals may comprise electrical current, voltage, impedance, resistance, capacitance, and/or conductance. Various techniques may be used for detecting target analytes, e.g., techniques from molecular biology (including recombinant techniques), cell biology (e.g., cell counting using a counting chamber (hemocytometer), plating methods, spectrophotometry, spectrometry (e.g., mass spectrometry), flow cytometry, Coulter counter etc.), immunoassay technology, microscopy (e.g., optical microscopy, fluorescent microscopy), image analysis, analytical chemistry, or combinations thereof.

In some cases, target analytes comprise one or more agents or moieties that may facilitate the detection. For example, the target analytes may comprise agents that may produce signals (e.g., light, or electrical signals). The agents may be associated with or bind to the target analytes. The agents may specifically bind to a particular type of target analytes. In some cases, the agents are antibodies that bind to a cell surface protein. The antibodies may comprise one or more detection agents which may produce signals, e.g., detection agents that may emit, scatter, reflect, deflect, or diffract light signals. In some examples, the target analytes may be treated (e.g., mixed) with one or more reagents. The treatment may occur prior to, during or after the separation is taking place. The one or more reagents may comprise stains. The stains may be any dye (e.g., a fluorescent dye), probe, substrate, or any chemical or biological substance that is suitable for staining a target analyte (e.g., a biological cell) or a portion thereof. The stains may enhance contrast and highlight structures of a stained object or a portion thereof. The stains may have a preference or specificity for a particular type of target analytes (e.g., a particular type of biological cells). In some cases, the stains mark (or stain) a given type of target analytes (or a portion thereof) in a particular color or fluorescence that is at least about 2, 3, 4, 5, 6, 7, 8, 9, 10 times greater in intensity than a staining intensity to another type of target analytes (or a portion thereof) at that same color or fluorescence spectrum.

The target analytes may be detected at a single molecule resolution. As an example, when the target analytes comprise cells such as senescent cells, the cells may be detected at a single cell resolution. The method may further comprise directing at least a portion of the target analytes from the microfluidic device to one or more analysis units for further analyses.

### Computer Systems

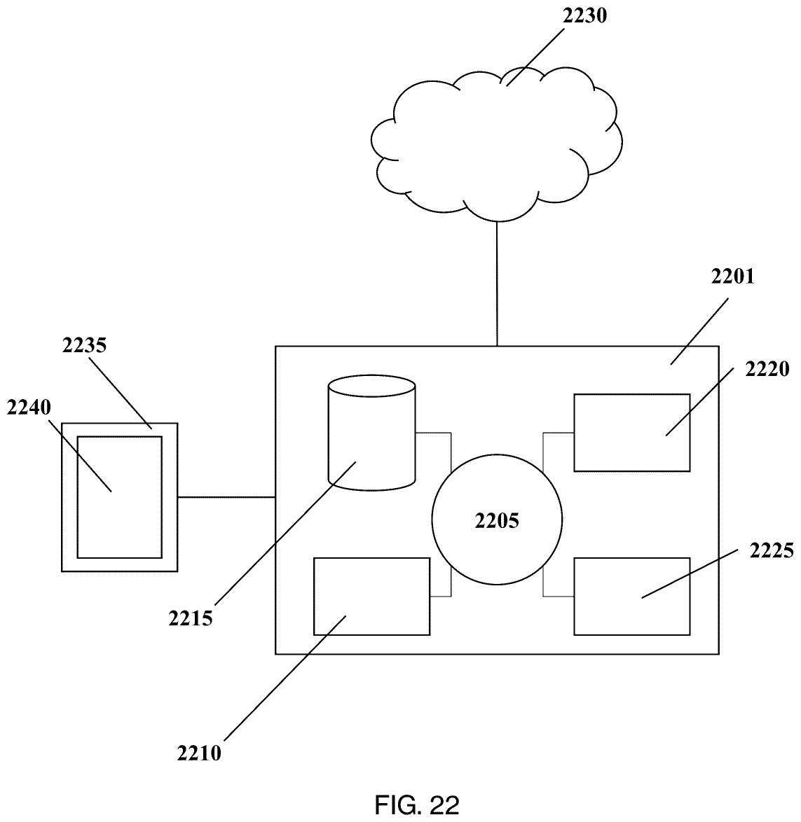

The present disclosure provides computer systems that are programmed to implement methods of the disclosure. FIG. 22 shows a computer system 2201 that is programmed or otherwise configured to perform various methods of the present disclosure. The computer system 2201 can regulate various aspects of methods and systems of the present disclosure, such as, for example, regulating fluid flow in a microfluidic device, adjusting flow rate of a fluid within a microfluidic device, directing a fluid to, from and/or through a microfluidic device. The computer system 2201 can be an electronic device of a user or a computer system that is remotely located with respect to the electronic device. The electronic device can be a mobile electronic device.

The computer system 2201 includes a central processing unit (CPU, also “processor” and “computer processor” herein) 2205, which can be a single core or multi core processor, or a plurality of processors for parallel processing. The computer system 2201 also includes memory or memory location 2210 (e.g., random-access memory, read-only memory, flash memory), electronic storage unit 2215 (e.g., hard disk), communication interface 2220 (e.g., network adapter) for communicating with one or more other systems, and peripheral devices 2225, such as cache, other memory, data storage and/or electronic display adapters. The memory 2210, storage unit 2215, interface 2220 and peripheral devices 2225 are in communication with the CPU 2205 through a communication bus (solid lines), such as a motherboard. The storage unit 2215 can be a data storage unit (or data repository) for storing data. The computer system 2201 can be operatively coupled to a computer network (“network”) 2230 with the aid of the communication interface 2220. The network 2230 can be the Internet, an internet and/or extranet, or an intranet and/or extranet that is in communication with the Internet. The network 2230 in some cases is a telecommunication and/or data network. The network 2230 can include one or more computer servers, which can enable distributed computing, such as cloud computing. The network 2230, in some cases with the aid of the computer system 2201, can implement a peer-to-peer network, which may enable devices coupled to the computer system 2201 to behave as a client or a server.

The CPU 2205 can execute a sequence of machine-readable instructions, which can be embodied in a program or software. The instructions may be stored in a memory location, such as the memory 2210. The instructions can be directed to the CPU 2205, which can subsequently program or otherwise configure the CPU 2205 to implement methods of the present disclosure. Examples of operations performed by the CPU 2205 can include fetch, decode, execute, and writeback.

The CPU 2205 can be part of a circuit, such as an integrated circuit. One or more other components of the system 2201 can be included in the circuit. In some cases, the circuit is an application specific integrated circuit (ASIC).

The storage unit 2215 can store files, such as drivers, libraries, and saved programs. The storage unit 2215 can store user data, e.g., user preferences and user programs. The computer system 2201 in some cases can include one or more additional data storage units that are external to the computer system 2201, such as located on a remote server that is in communication with the computer system 2201 through an intranet or the Internet.

The computer system 2201 can communicate with one or more remote computer systems through the network 2230. For instance, the computer system 2201 can communicate with a remote computer system of a user (e.g., a lab technician, a physician). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 2201 via the network 2230.

Methods as described herein can be implemented by way of machine (e.g., computer processor) executable code stored on an electronic storage location of the computer system 1101, such as, for example, on the memory 2210 or electronic storage unit 2215. The machine executable or machine readable code can be provided in the form of software. During use, the code can be executed by the processor 2205. In some cases, the code can be retrieved from the storage unit 2215 and stored on the memory 2210 for ready access by the processor 2205. In some situations, the electronic storage unit 2215 can be precluded, and machine-executable instructions are stored on memory 2210.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 2201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2201 can include or be in communication with an electronic display 2235 that comprises a user interface (UI) 2240 for providing, for example, parameters and/or information of microfluidic devices, or instructions for handling one or more samples. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2205. The algorithm can, for example, perform methods of the present disclosure.

### EXAMPLES

Chips and manufacturing thereof. The Polydimethylsiloxane (PDMS) microfluidic channel was fabricated with soft lithography. The mask was designed with the AutoCAD software (Autodesk Inc., San Rafael, CA) and produced by Photo Sciences, Inc. (Torrance, CA). The silicon master as a PDMS mold was produced by standard photolithography and deep reactive ion etching (DRIE) techniques. To fabricate the PDMS mold, 2 μm g-line photoresist (FujiFilm, USA) was coated on a 6-inch silicon wafer by spin coating. Followed with UV exposure to transfer the pattern from a mask to the photoresist layer, the silicon wafer was developed to generate a pattern on photoresist. After hard-bake, the wafer was etched by DRIE to produce channels with the desired depth. For the chip, the channel depth was controlled between 30 and 35 μm. Finally, a Teflon layer was deposited on all surfaces of the silicon wafer to ensure a smooth PDMS peeling-off process. The chip was prepared by bonding the PDMS-replica channel onto a glass slide after treated with plasma (PDC-001, Harrick Plasma, USA). For the parallel-processing chip, five layers of identical PDMS channels were stacked up with the inlets and outlets aligned along the vertical direction. Before use, the device was incubated at 100° C. overnight to prevent the fluid leakage and confirmed by the flow-through of 1× PBS buffer. Microtubing with 0.8 mm ID and 1.4 mm OD (Cole-Parmer, USA) was connected to the PDMS channels for fluid delivery. The devices were disposed after each run of biological samples.

Assembly and quality control of a chip platform. An epifluorescence microscope (IX83, Olympus, Japan) connected with a CCD camera (QIClick, QImaging, Canada) was used to observe and record the cell separation process inside the microfluidic channel. The blood sample and 1×PBS buffer with 0.05% BSA were stored in 3 mL and 50 mL syringes (BD Biosciences, USA), respectively. A 0.45 μm syringe filter (Acrodisc, Pall Life Sciences, USA) was connected to the syringe storing the PBS buffer to prevent contaminant from flowing into the microchannel and clogging the filter array (or obstacle array). Two infusion syringe pumps (NE-1600, New Era Pump Systems, USA; and KDS 100, KD Scientific, USA) were used to control the flow rates. When the chip was tested with cells spiked in the undiluted whole blood, the buffer flow rate was usually 3 times of that of the blood sample. This ratio was decreased to 2 when the chip was tested with low concentration of cells or beads solution alone. Due to the sedimentation of cells, the syringe containing blood sample was vertically positioned to ensure the most of the cells flowed into the microtubing. When the chip contains only one outlet with capture arrays for cell trapping, a much longer microtubing was connected to the other outlet without capture arrays to balance the hydrodynamic resistance through both outlets. Before separation, 1xPBS buffer with 0.05% BSA flowed through the microchannel and microtubing for ˜15 min to remove any remaining air bubbles and reduce nonspecific bonding to the channels. For the portable detection of MSCs on the chip, an iPhone 6 smart phone connected with a 60-100× mobile phone microscope lens (Neewer, China) was used to take and visualize the images.

Preparation of samples for validating chips. Fresh human whole blood from healthy donors collected within 24 h was purchased from AllCells Inc. (Alameda, CA). The blood samples were collected with K2EDTA blood collection tubes (BD Biosciences, USA). Polystyrene beads with varied sizes were purchased from Polysciences, Inc. (Warminster, PA) and Bangs Laboratories, Inc (Fishers, IN). Coulter Z2 cell counter (Beckman Coulter, USA), BioRad TC20 cell counter (BioRad, USA), and a hemocytometer (Hausser Scientific, USA) were used to measure the number and concentration of cells and polystyrene beads and for cross validations. Human mesenchymal stem cells (MSCs) were purchased from Lonza (Lonza, Swiss). The log number used in this study was 0000471980 (derived from a 20-year-old male). MSCs were maintained in humidified incubators at 37° C. with 5% CO₂, and cultured with MSCs basal medium (Lonza) supplanted with 5% FBS. MSCs at passage 6 were cultured on 12-well plates with proper densities to avoid over confluency over a 6-day period. The initial number of cells for each condition is shown in Table 1 and Table 2. For hydrogen peroxide (H₂O₂) treatment, 30% H₂O₂ solution (Sigma, USA) were diluted with MSCs basal medium into desired concentrations. Media containing 100 μM and 200 μM H₂O₂ as well as the basal medium control were used to incubate MSCs at 37° C. for 2 h. After that, the MSCs were washed with 1x× PBS solution 3 times and cultured in the fresh media for another 3 days before analysis. For X-ray treatment, MSCs were placed on a rotating table and exposed to 1 Gy, 4 Gy or sham (0 Gy), using a RAD320 320 kVp X-ray machine (Precision X-ray Inc., North Branford, CT), operated at 300 kV, 10 mA (dose rate of 1.3 Gy/min). Cells were cultured for another 3 days and 6 days, respectively, before analysis.

TABLE 1
Initial number of MSCs on a well for hydrogen peroxide treatment

| | 0 μM | 100 μM | 200 μM |
|---|---|---|---|
| 3 | 1 × 10⁴ | 1.5 × 10⁴ | 2 × 10⁴ |
| Days | cells/well | cells/well | cells/well |

TABLE 2
Initial number of MSCs on a well for X-ray irradiation

| | 0 Gy | 1 Gy | 4 Gy |
|---|---|---|---|
| 3 | 1 × 10⁴ | 2 × 10⁴ | 4 × 10⁴ |
| Days | cells/well | cells/well | cells/well |
| 6 | 5 × 10³ | 1 × 10⁴ | 2 × 10⁴ |
| Days | cells/well | cells/well | cells/well |

For mouse bone marrow samples, 10 weeks old, male wild-type mice (stain C57BL/6) were exposed to the total body X-ray irradiation at 0 Gy (sham), 1 Gy, 4 Gy, and 6.5 Gy, with 4 mice at each dose, respectively. The bone marrow samples were collected 10 days after the X-ray treatment and diluted with 1× PBS buffer to a total volume of ˜1.5 mL per mouse. For cell separation study, MSCs were dissociated with trypsin (Lonza, Swiss), fixed, and stored in 1x PBS buffer. Before being processed with a chip, the whole blood sample spiked with MSCs was filtered with a 40 μm cell strainer (Falcon, Corning, USA) to remove contaminants and clotting. To identify senescent MSCs, a Senescence Detection Kit (BioVision, CA) was used to stain senescent cells into blue color. To stain suspended MSCs, cells were incubated with the staining solution on chip or inside a tube at 37° C. overnight before study. For WBC study, 10 mL human whole blood was added to 200 mL 1× RBC lysis buffer (BioLegend, San Diego, CA) and incubated at room temperature for 15 min, followed by a centrifugation at 350×g for 5 min to enrich WBCs. The isolated WBCs were resuspended with 10 mL 1× PBS buffer and used to characterize the chips. The concentration of the input WBCs and recovered WBCs was measured with a Bio-Rad cell counter. To differentiate WBCs from the RBCs background, the nucleus of WBC was stained with Hoechst 33342 (Thermo Fisher Scientific, USA) and observed under a fluorescence microscope (350/461, DAPI). PE-CF594 mouse anti-human CD-45 antibodies (BD Horizon, USA) were used to label WBCs for fluorescence imaging.

Operation of chips. (1) A sample chip for analysis of senescent cells in whole blood. The chip with a 4 μm 3D filter array (or obstacle array) and a cell trapping array was used to isolate MSCs from whole blood, capture MSCs on chip, and conduct single cell analysis in situ after capture. 2 mL of fresh undiluted human whole blood spiked with ˜500 fixed senescent MSCs induced by either H₂O₂- or X-ray was injected into the chip at a flow rate of 3 mL/h. 1×PBS buffer with 0.05% BSA was injected from another inlet at a flow rate of 9 mL/h. After MSCs were captured on the cell trapping array, the flows of cell sample and buffer were stopped, and followed by a gentle injection of staining solution to fill the whole channel and tubing. The inlet tubing was kept during incubation to generate a balance pressure and prevent backflow of the trapped cells. During the separation and staining processes, air bubbles should be avoided inside the channel. After incubation, color images of the captured MSCs were recorded with the microscope for analysis. (2) A sample chip for removal of senescent cells from whole blood. A chip with a 13 μm 3D filter array (or obstacle array) was used to remove senescent MSCs from blood. Before spiked into human whole blood, the fixed senescent MSCs induced by either H₂O₂ or X-ray were stained overnight with the Senescence Detection Kit in a centrifuge tube. The percentage of the senescent MSCs was manually counted under the microscope by dropping 10 μL of the stained MSC sample on a glass slides. Then ˜10,000 stained MSCs were spiked into 3 mL undiluted human whole blood and run through the device at a flow rate of 3 mL/h. For the high-throughput separation device, the flow rate was increased to 300 mL/h. The removed senescent MSCs were collected in a tube from the outlet to measure the cell numbers and the percentage of senescent MSCs. The removed senescent MSCs were collected in a tube from the outlet to measure the cell concentration and the percentage of senescent MSCs. Therefore, the number of input and output senescent cells could be calculated. The removal rate was then determined by the ratio of output senescent MSCs over to input senescent MSCs.

Data analysis for chips. (1) Quantification of senescent cells on cell culture plates. To quantify the senescent MSCs on cell culture plates, the MSCs were fixed and then stained with Senescence Detection Kit and Hoechst 33342 (Thermo Fisher Scientific, USA). For each sample, five regions were randomly picked and recorded as a color image (RGB mode) and a fluorescent image (350/461, DAPI) using CCD camera on microscope with a 10× objective. The number of total MSCs and senescent MSCs were manually counted from the fluorescent images (DAPI) and color images (blue stain), respectively. Therefore, the percentage of senescent MSCs in each sample was determined by the ratio of senescent MSC number to total MSC number. (2) Quantification of senescent cells on chips. After staining the MSCs on chip overnight, the color images of MSCs were recorded with a CCD camera in RGB mode. Microscope lamp intensity was consistent at 5 V. The images were then imported into ImageJ software to isolate their red channels, which were used to identify senescent MSCs. The grayscale of the dark region for each cell was measured with ImageJ, which define the senescent MSCs with a value smaller than 40.

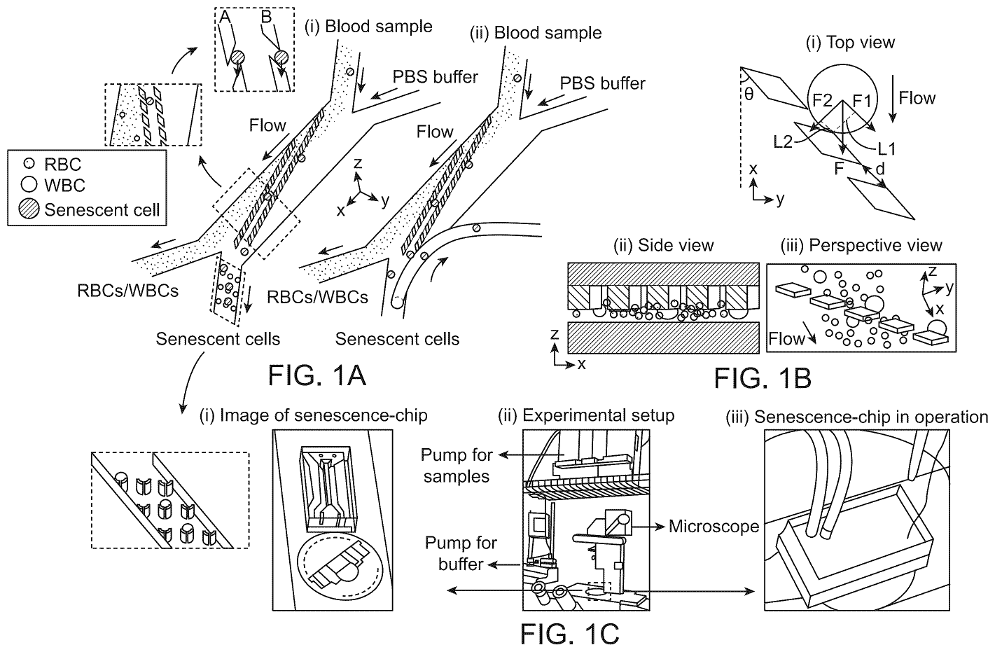

Mechanism of chips. FIGS. 1A-1C illustrate sample chips which monolithically integrate two rows of a tilted 3D filter array of obstacles for size-based cell separation with all necessary inlets and outlets for samples and buffers. Two types of chips were made for different purposes. For analysis of senescent cells in small volumes of whole blood or bone marrow, the chip contains a 3D-filter array (or obstacle array) to isolate MSCs, followed with a cell trap array to capture MSCs after separation for enumeration and single cell analysis of senescent cells (FIG. 1A(i)). For rapid removal of senescent cells from whole blood, the chip may not contain cell traps but a fluid outlet may be connected directly to a tubing to remove senescent cells from whole blood (FIG. 1A(ii)). The other end of the tubing may be connected to a waste or a collection tube for further analyses.

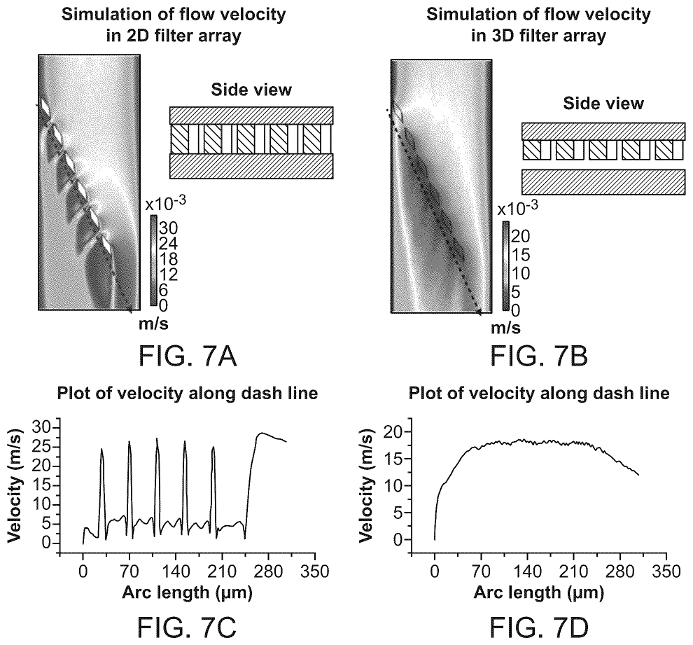

Modeling to optimize the configuration of the chips were shown in FIG. 1B and FIGS. 7A-7D. A 3D filter array (or obstacle array) is fabricated with PDMS micropillars inside the channel for cell separation on the x-y plane as well as in the z direction. On the x-y plane, two key parameters may be taken into consideration, which are the inclination angle (θ) of micropillars relative to the fluid flow, and the inter-pillar spacing (d) as shown in FIG. 1B(i). The pillar shape was also optimized to minimize clogging and maximize cell separation. Compared to the circular and triangle pillars, quadrangle pillars may have more uniformity in terms of flow pressure, deformability, and z-direction opening. Two types of quadrangle pillars were manufactured as shown in the zoom-in of FIG. 1A. When moving down the filter arrays (or obstacle arrays), the particle may be trapped by the sharp edge of the Type-A pillars. For Type-B pillars, the particle may contact a tilted surface on the pillars and may be easier to move on. Therefore, the Type-B pillars may have a better performance in particle separation. For the rigid particles with diameters smaller than the pillar spacing (d), they may directly pass through the filter (or obstacle). When a particle has a diameter larger than the pillar spacing, the hydrodynamic drag force (F) may be divided into two portions, parallel to the filter (F₁) and perpendicular to the filter (F₂). To ensure that the particles may roll down on the filter, the relation

$${F}_{1} \cdot {L}_{1} > {F}_{2} \cdot {L}_{2} \qquad ( 1 )$$

may be established, in which L₁ and L₂ are the arms of forces F₁ and F₂. In Eq. (1), F₁ and F₂ may be expressed as F·cos θ and F·sin θ, while L₁ and L₂ may be expressed as (R²−1/4d²)^{1/2} and 1/2d, in which R is the radius of the particle. Therefore, Eq. (1) could be expressed as

$$d < 2 R / {( {tan}^{2} θ + 1 )}^{1 / 2} \qquad ( 2 )$$

From Eq. 2, a smaller pillar spacing (d) and filter angle (θ) may help particles to roll down on the pillars as shown in FIG. 1A. In some cases, the angle of filter array (θ) may be 5°. As provided above and elsewhere herein, the pillar spacing (d) (also called “filter size” or “obstacle size”) may be varied based on the size of particles to be isolated.

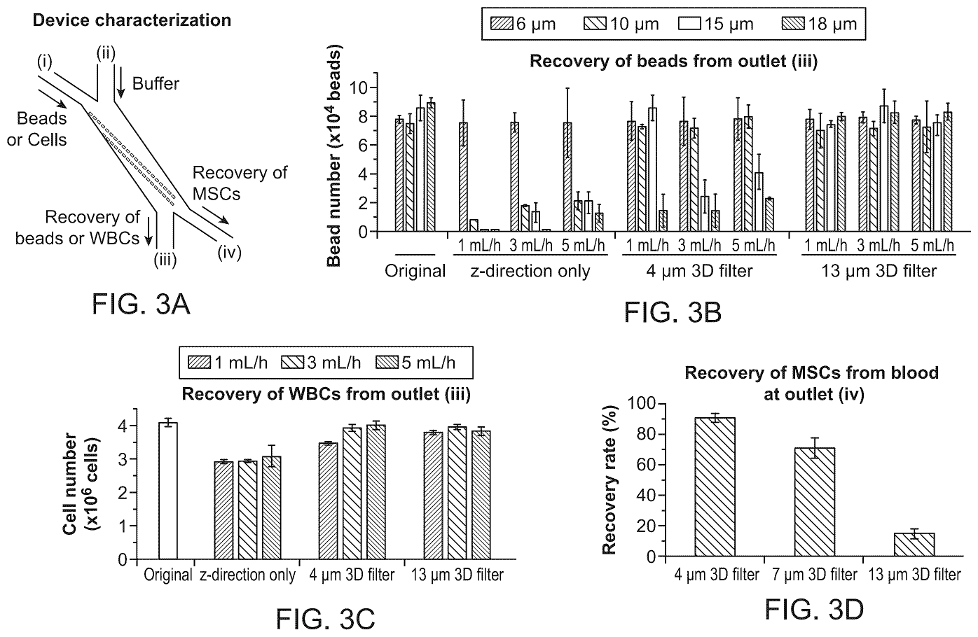

In the z direction, the PDMS pillars may not bond to the glass substrate because of their small top-surface area. Therefore, depending on the operational flow rate in the channel, an opening with varying size may be created between the pillars and the glass substrate, which may work as a shutter and allow for smaller particles (e.g., cells) to pass through (FIG. 1B). For example, during the separation of MSCs from whole blood, RBCs and WBCs can easily pass through the filter from both the z-direction and x-y plane, while the MSCs with a larger size may not cross the filter but instead roll down. As shown in FIGS. 7A-7D, as compared to the 2D filter array (or obstacle array) (FIGS. 7A and 7C), 3D filter array (FIGS. 7B and 7D) may generate much more uniform flow velocity across the channel. Therefore, the 3D filter array may better reduce the system backpressure, reduce clogging of the filter, and improve the throughput.

FIG. 1C shows the experimental setup for the operation of a sample chip. Two syringe pumps are used to deliver the 1× PBS buffer and blood samples into two inlets, respectively (FIG. 1C(ii)). A sheath flow of 1× PBS buffer may ensure the blood sample flow into the left outlet. As the cells in the blood sample flow down to the main channel, smaller cells such as RBCs and WBCs may pass the 3D filter array without changing their flow path. As a result, the smaller cells may exit to the left outlet. However, larger cells such as MSCs may be filtered out by the filters and roll down following the pillars to the right outlet (FIG. 1C(iii)).

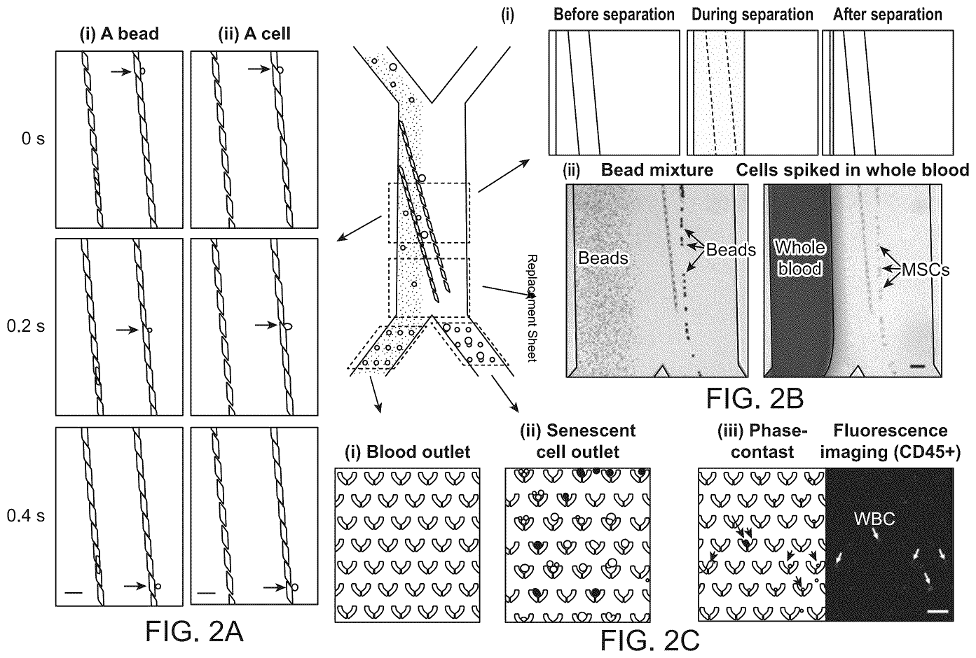

Performance of chips. The performance of sample chips is tested and testing results are shown in FIGS. 2A-2C. On a 4 μm 3D filter array (or obstacle array) (d=4 μm), time-lapse images clearly demonstrated that a 15 μm bead and a MSC rolled down on the filter array (FIG. 2A). In addition, when undiluted whole blood sample passed through the 3D filter array, no clogging was observed (FIG. 2B(i)). The separation ability of the chip with mixtures of polystyrene microbeads and MSCs spiked into undiluted fresh human whole blood is established (FIG. 2B(ii)). When 10 μm and 18 μm beads flowed down the channel, only the 18 μm particles may be filtered out by the filter while the smaller beads crossed the filter, as shown in the stacking image of FIG. 2B(ii) (left). After filtration, the 18 μm particles moving along the filter array were collected downstream from the right outlet. Similarly, the 4 μm 3D filter may isolate MSCs from blood cells, as shown in the stacking image of FIG. 2B(ii) (right). Although basal and senescent human MSCs may be heterogeneous in size, the microfluidic chip may still isolate most of them from blood cells due to their larger average sizes than RBCs and WBCs. After separation, MSCs were captured by the cell trap array located at the device outlet (FIG. 2C). On-chip SA-β-gal staining showed good separation of senescent MSCs (right outlet) and almost no background MSCs in blood cells (left outlet) (FIG. 2C(i)-(ii)). After labeled with anti-CD45 antibodies, background WBCs were able to be identified and excluded by comparing the phase-contrast image to fluorescence image (FIG. 2C(iii)).

Validation of chips. To further validate the separation ability of the chip and its dependence on 3D filter sizes (or obstacle sizes) and flow rates, device characterization using bead mixtures, isolated WBCs, and basal MSCs spiked in whole blood was performed (FIGS. 3A-3D). A mixture of beads and cells was received from the inlet (i) and the beads and cells which passed through the filter (or obstacle) from both the outlet (iii) and (iv) were recovered (FIG. 3A). The numbers of recovered beads and cells were measured. The bead mixture contained roughly an equal mix of 4 different sizes (6 μm, 10 μm, 15 μm, and 18 μm), and the total numbers of beads for each size was around 8.0-9.0×10⁴. As shown in FIG. 3B, most of the beads larger than 10 μm were removed by the z-direction only filter array (dam-like, with no openings in the x and y directions), while only 6 μm beads may pass the filter. As the flow rate increased from 1 mL/h to 5 mL/h, the number of beads larger than 10 μm also slightly increased, but was still lower than 25% of the original concentration. The results indicate that in 3D filter array, the spacing along the z-direction was smaller than 10 μm, but it may increase as the flow rate increased. With the 4 μm 3D filter array, more than 90% of the 6 μm and 10 μm beads may pass through the filter to outlet (iii), while larger particles were removed, suggesting the effective spacing along the z-direction was increased in the presence of openings in the x and y directions. As the pillar spacing increased to 13 μm, all size of beads from 6 μm to 18 μm may be recovered from outlet (iii), independent of the flow rates. Majority of the WBCs have a size between 8-12 μm. To test the ability of the chip to recover WBCs while removing senescent MSCs from whole blood, the RBC-lysed blood sample were used. The original (input) cell number of WBCs was around 4×10⁶. As shown in FIG. 3C, the z-direction only filter array allowed ˜75% of the WBCs to pass, while for the 4 μm and 13 μm 3D filter arrays, almost all of the WBCs may pass through and be recovered from outlet (iii). No WBCs were observed in cell traps at outlet (iv), confirmed by negative immunostaining with CD45. Presumably, the smaller WBCs were filtered through to the outlet (iii) or passed through the gap between the cell traps (˜10 μm) at outlet (iv), while the giant WBCs were pre-filtered by 40 μm cell strainer prior to on-chip separation.

Basal MSCs were spiked in whole blood and the original input number of MSCs was approximately 1×10⁴. The number of recovered MSCs was measured at outlet (iv) and the recovery rate was calculated, which is defined as the ratio between the recovered MSC number to the input MSC number. As shown in FIG. 3D, three types of 3D filter arrays (or obstacle arrays) with different filter sizes (pillar spacing of 4 μm, 7 μm, and 13 μm) were tested at a flow rate of 3 mL/h. The recovery rate of basal MSCs at outlet (iv) dropped from ˜90% to ˜20% as the 3D filter size increased from 4 μm to 13 μm.

Based on the results, 4 μm 3D filter arrays were used for analysis of senescent cells. Such filter size may isolate most of the MSCs from whole blood containing most of RBCs and WBCs, which may allow for quantification of the numbers and percentage of senescent MSCs among total MSCs. For removal of senescent cells from whole blood, it may be desirable to maximize the recovery of basal MSCs from outlet (iii) while only selecting senescent MSCs to outlet (iv), therefore 13 μm filter size may be utilized in this application.

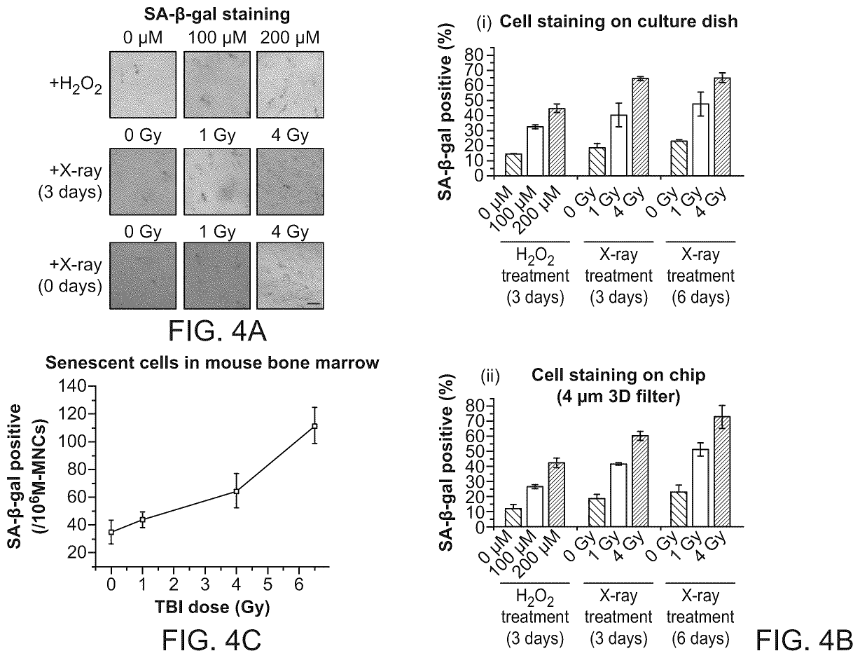

The microfluidic devices of the present disclosure may be used for analysis of senescent cells in human whole blood and mouse bone marrow samples (FIGS. 4A-4C). MSCs have been demonstrated to undergo in vitro cellular senescence by treatment of hydrogen peroxide and irradiation of X-ray. MSCs were treated with different doses of hydrogen peroxide (H₂O₂, 0, 100, 200 μM) and X-ray (0, 1, 4 Gy), and analyzed 3 days and 6 days after the treatments. Both dose-dependent and day-dependent increases of the percentage of SA-β-gal positive (stained blue) MSCs were observed on 12-well cell plates (FIG. 4A and FIG. 4B(i)). The H₂O₂- and X-ray-induced senescent MSCs (˜500 cells) were spiked in undiluted whole blood (˜2 mL) and underwent separation on the chip with a 4 μm 3D filter array at 3 mL/h, captured and stained on the single cell traps at outlet. Dose-and day-dependent increases of the percentage of SA-β-gal positive MSCs on the cell trap were observed, matching those determined by direct cell staining on culture dish (FIG. 4B(ii)). Importantly, the quantitation by the chips was achieved by starting with small numbers of MSCs and in the presence of undiluted human whole blood.

In some examples, the microfluidic devices (such as a microfluidic chip) may be used for separating or isolating senescent cells from mouse bone marrow. Four groups of mice were exposed to different doses of X-ray (0, 1, 4, 6.5 Gy). 10 days after TBI, the bone marrow was obtained and diluted into 1.5 mL with 1× PBS. About 1×10⁶ bone marrow mononuclear cells (BM-MNCs) from each sample was aliquoted and diluted into 2 mL for cell separation directly on the chips. As shown in FIG. 4C, the number of senescent cells isolated by the chips increased from the average 34 to average 112 as IR dose increased. Thus, the chips may be able to isolate and enumerate senescent cells from small volumes of various biofluids.

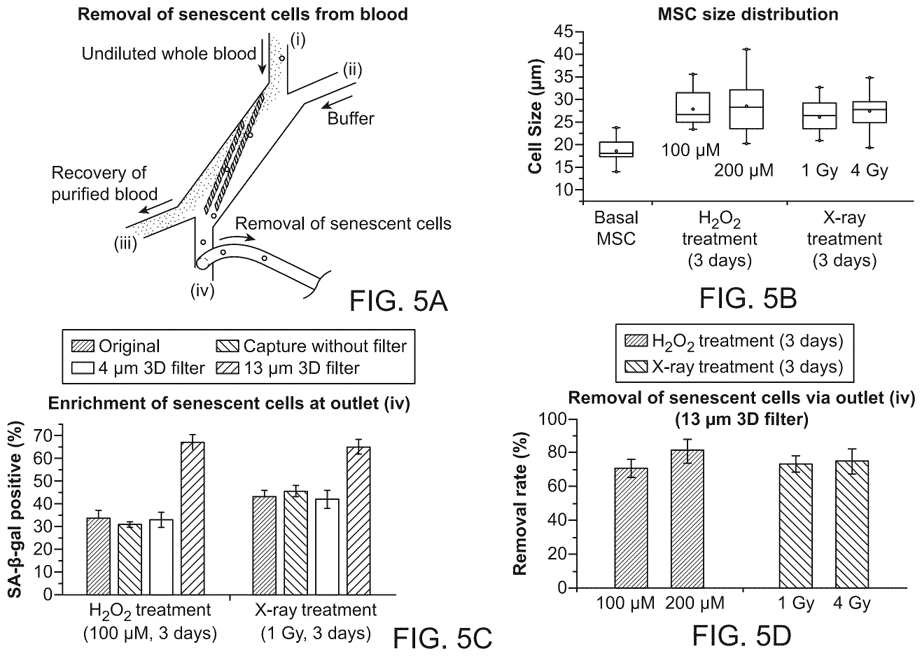

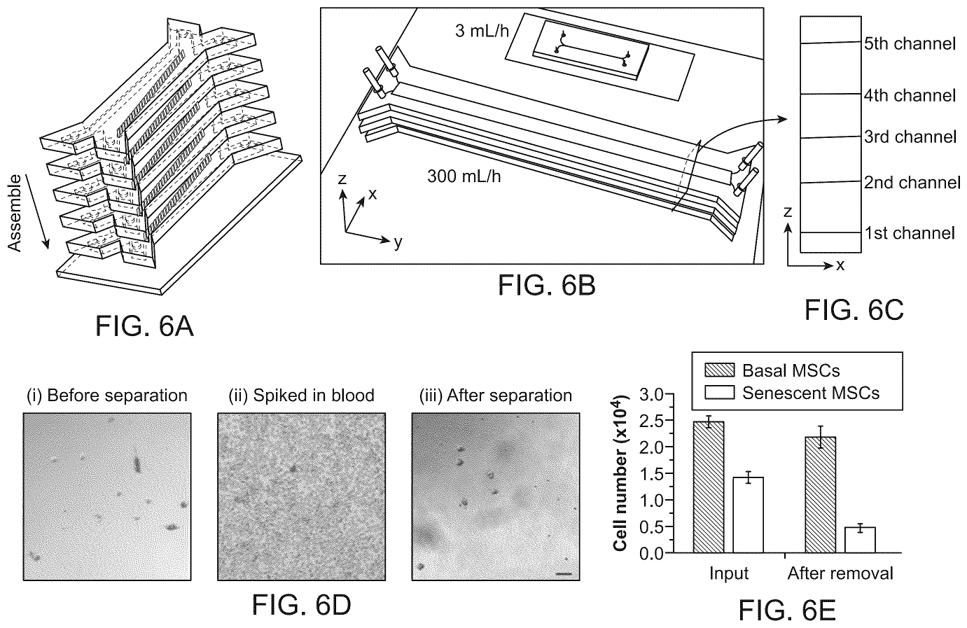

The microfluidic devices of the present disclosure may be used for removal of senescent cells from whole blood samples for potential therapeutic targeting of cellular senescence (FIGS. 5A-5D and FIGS. 6A-6E). The overall strategy was to maximize recovery of the major blood components including plasma, RBCs, WBCs, and healthy cells (in this case basal MSCs) from the outlet (iii), while removing most of the senescent MSCs through the outlet (iv) (FIG. 5A). To choose the filter size of 3D filter array for this application, it was determined that the average cell size of basal MSCs to be 18 μm and that of senescent MSCs to be above 25 μm (FIG. 5B). The enrichment efficiency by separating senescent MSCs from basal MSCs and captured them at outlet (iv) using 4 μm and 13 μm 3D filter arrays were shown in FIG. 5C. A control experiment was carried out by directly flowing the MSCs through the cell trap array to capture cells without a 3D filter. A significantly higher percentage (˜65%) of SA-β-gal positive MSCs were found at the outlet (iv) for 13 μm 3D filter array than the others. The result was consistent with those shown in FIG. 3D, where more than 80% of the basal MSCs were able to pass through the 13 μm 3D filter and recovered from outlet (iii). Therefore, the chip with a 13 μm 3D filter array was used for selective removal of senescent MSCs from basal MSCs and blood components. At a flow rate of 3 mL/h on this single-unit small-size chip, over 70% removal of pre-stained and SA-β-gal positive MSCs was achieved for both hydrogen peroxide and X-ray-induced senescent MSCs (FIG. 5D).

The microfluidic devices of the present disclosure may also be used to process samples with a higher throughput. As shown in FIGS. 6A-6C, the chip dimension may be scaled up by 10 folds. For example, the channel width may be increased from ˜10³ μm to ˜10⁴ μm. To prevent the deformation and collapse of the wide PDMS channel under high flow pressure, the chip was fabricated with posts uniformly distributed inside the channel. Five devices were stacked along the vertical direction for parallel processing to further improve the throughput. The multiplexed chips shared the same inlets and outlets to keep the operation simple. Given the dominant hydrodynamic resistance by the filter array over inlets and outlets, uniform flow rates were applied on each of five chips. With this multiplexed system, the throughput was increased by two orders of magnitude from 3 mL/h to 300 mL/h. To characterize the performance of the ultrahigh-throughput chip, 4×10⁴ pre-stained MSCs containing both senescent and non-senescent cells were spiked into 30 mL undiluted human whole blood for separation. As shown in FIGS. 6D and 6E, after separation, more than 70% of the senescent MSCs were removed while less than 15% of the basal MSCs were removed, demonstrating the similar performance from the high-throughput multi-unit chip as the single-unit small-size chip.

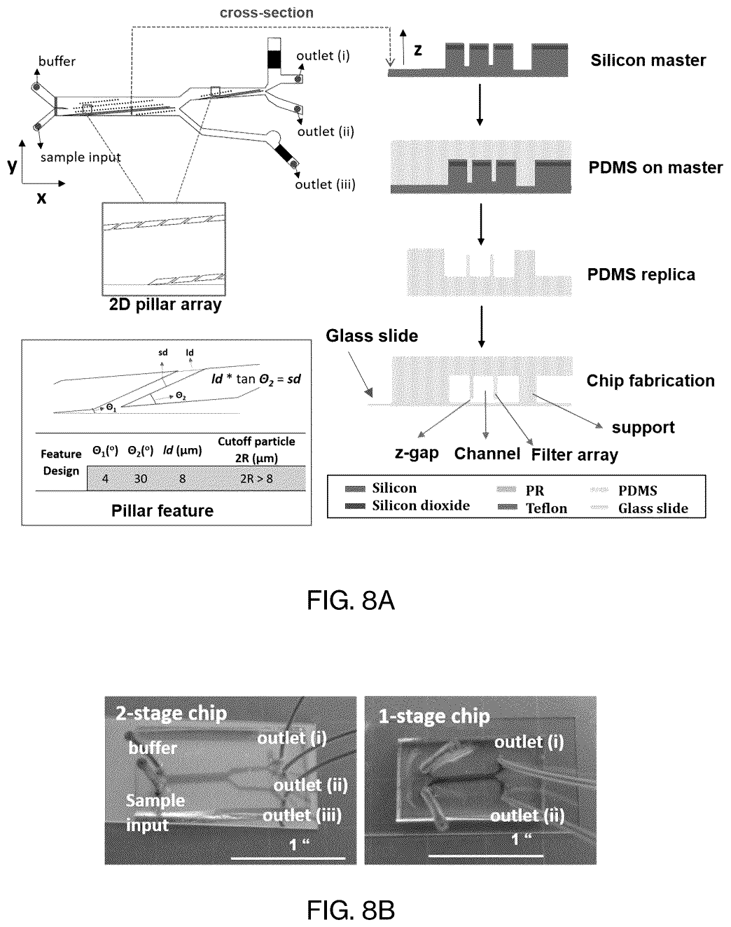

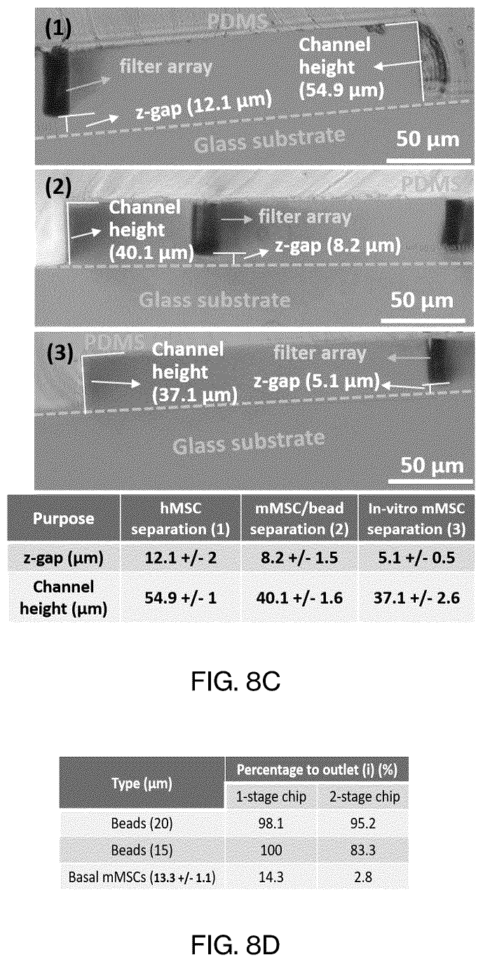

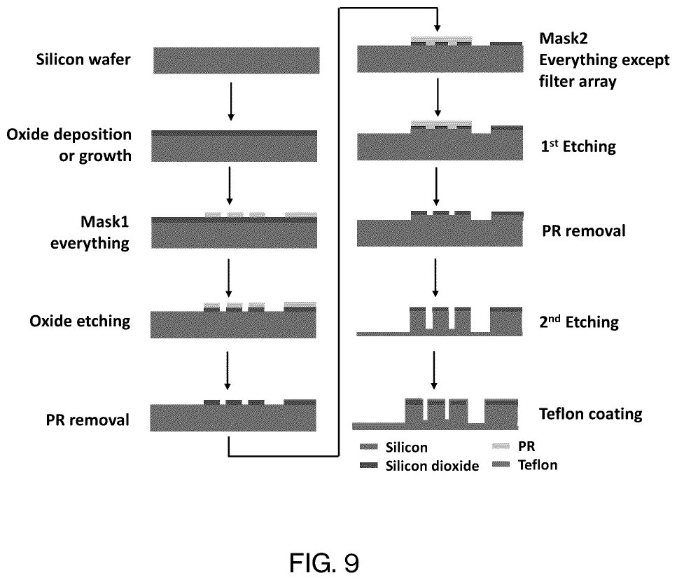

Device design and fabrication. 1-stage (e.g., comprising one array of obstacles with a certain vertical spacing) or 2-stage (e.g., comprising two array of obstacles each with a certain vertical spacing) devices of the present disclosure (FIGS. 8A-8D) were fabricated, each comprising a slanted filter array with an inclination angle relative to the fluidic flow within the chip, the height of the chip is 37, 40, or 55 μm depending on the cell types. The chip comprised two inlets and two outlets for the 1-stage device, two inlets and three outlets for the 2-stage device. An array of circular posts with a diameter of 80 μm and a gap of 80 μm between posts may be designed before the filter array to prevent large debris from entering the functioning module of the chip. All silicon master was fabricated by standard photolithography including lithography, oxide deposition, Teflon deposition, and dry etching information. The process scheme to fabricate the silicon master is shown in FIG. 9. The fabrication features two lithography processes to create an etching depth difference between the filter array features and the channel structures. Polydimethylsiloxane (PDMS) soft lithography techniques were then applied to fabricate the working chips. Polystyrene beads with varied sizes were purchased from Polysciences, Inc. (Warminster, PA) and Bangs Laboratories, Inc (Fishers, IN) and were used to validate the separation performance of device.

### Silicon Mold Fabrication, Device Fabrication, and Device Validation

De The PDMS (polydimethylsiloxane) device was fabricated with soft lithography. The mask was designed with AutoCAD (Autodesk Inc., San Rafael, CA) and produced by Photo Sciences, Inc. (Torrance, CA). A simulation of the flow velocity, shear stress, and particle tracking inside the channel was conducted with COMSOL Multiphysics modeling software (Palo Alto, CA) for microfluidics design. The silicon master as a PDMS mold was produced by standard photolithography and deep reactive ion etching (DRIE) techniques. To fabricate the PDMS mold, a 2 μm g-line photoresist (FujiFilm, USA) was coated on a 6-inch silicon wafer by spin coating. Silicon molds were exposed to UV to transfer the pattern from a mask to the photoresist layer, the silicon wafer was developed to generate a pattern on the photoresist. After hard-bake, the wafer was etched by DRIE to produce trenches and channels with the desired depth. For the device, the channel depth was controlled from 37 to 51 μm, and the z-gap of the filter array was controlled from 5 to 12 μm. Finally, a Teflon layer was deposited on all surfaces of the silicon wafer to ensure a smooth PDMS peeling-off process. The details of the fabrication processes are shown in FIG. 9. The height of the z-gap was achieved by the two etching processes, where the trenches were etched only after lithography using the 2^{nd} mask, while the channels were etched after both lithographies using 1^{st} and 2^{nd} masks. After the consolidation of PDMS on the silicon master, the PDMS replica was peeled off from the silicon master. The PDMS replica and a glass slide were treated by a plasma etcher (PDC-001, Harrick Plasma, USA) at 1000 mtorr for 90 seconds before bonding the PDMS replica and glass slide together. The bound device was heated in an oven (10-140, Quincy Lab, Inc., USA) at 60° C. for 1 hour to enhance the bonding strength. After chip bonding, the device was treated with the same plasma etcher to activate the inner surface at the same conditions for 120 sec. The interior surface of the device was incubated with DPBS containing 1 wt. % Pluronic F-68 polyol (61-161-RM, or Corning™ Poloxamer 188, Corning, USA) for at least 1 day at 4° C. to passivate the surface to reduce nonspecific binding. For validation of device size separation performance, 15 μm beads dispersed in 1 wt % F-68 in DPBS were injected into the device with z-gap ˜8.2 μm. As expected, 15 μm beads cannot flow through the z-gap of the filter array, instead, beads roll over the filter array on the x-y plane.

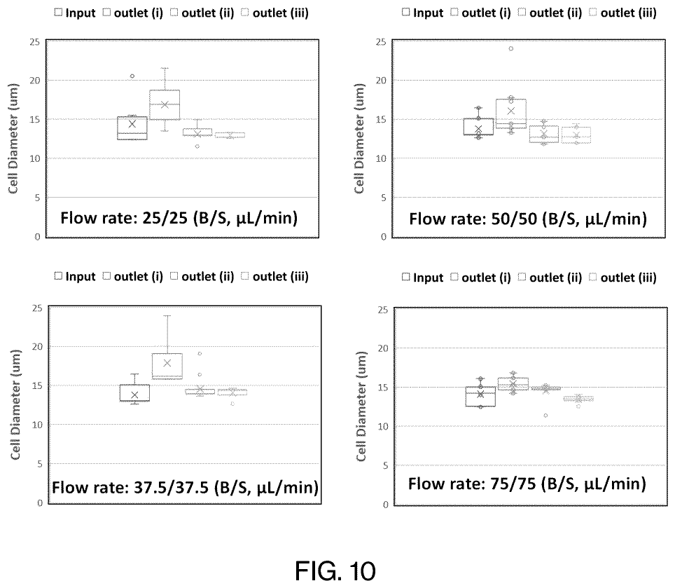

Optimization of operational flow rates. Several flow rate combinations for buffer and sample inlets (25/25, 37.5/37.5, 50/50, 75/75 L/min for buffer/sample (B/S) inlets) were investigated for the optimized flow rates to run cells in the device using mouse MSCs. The separation efficiency in terms of cell number is similar for the studied four flow rate combinations, however, the cell size distribution acquired from each outlet under different flow rates is very different as shown in FIG. 10. The average cell size in solution acquired from the outlet (i) was larger than that from the outlet (ii) and (iii) except when the device was operated at 75/75 (B/S) μL/min flow rates. At 75/75 (B/S) μL/min flow rate, an increase in cell debris was observed, likely due to cell disintegration from shear stress applied to cells. For the other three flow rate combinations, the cell size distribution acquired from the outlet (ii) or (iii) became wider as the flow rate increased. For optimal cell separation resolution, a narrower cell size distribution was selected to minimize the overlay between cell populations. The flow rate of 25/25 (B/S) μL/min was chosen as the operating flow rate when running the cell samples.

Experimental setup. An epifluorescence microscope (IX83, Olympus, Japan), connected with a CCD camera (QIClick, QImaging, Canada), and controlled by Olympus cellSens Dimension™ software, was used to observe and record the motion of the beads or cell separation process and to take images of beads or cells inside the device. A three-channel peristaltic pump (EW-78001-70, Cole-Parmer, USA) was used to control the fluidic system. The flow rates pumping to two inlets were 25 μL/min. Several other flow rates were also investigated, and the cell size distribution under different flow rates is shown in FIG. 10. Dulbecco's phosphate-buffered saline (DPBS, 21-031-CV, Corning, USA) was used as diluent and buffer inlet when analyzing beads in the device, while for cell or mouse-derived bone marrow samples, cell culture media was used in the buffer inlet. All tubes connected to inlets or outlets were incubated with cell medium for 10 minutes before sample loading to reduce non-specific binding. The number of cells or polystyrene beads was visualized under the microscope and counted by a BioRad TC20 cell counter (BioRad, USA).

Cell culture and staining. Human mesenchymal stem cells (MSCs) were derived from a 20-year-old male and purchased from Lonza (Lonza, 0000471980). Mouse mesenchymal stem cells (MSCs) were purchased from Cyagen (Strain C57BL/6 Mouse Mesenchymal Stem Cells, MUBMX-01001, Cyagen, USA). Cells were cultured in a humidified incubator (Symphony 5.3A CO₂ incubator, VWR, USA) at 37° C. with 5% CO₂. MSCs basal medium (PT-3238, Lonza) with MSCGM™ SingleQuots supplement kit (PT-4105, Lonza) was used for hMSC culture, and MesenCult™ Expansion kit (5513, STEMCELL Technologies, Canada) was used for mMSC culture. Both media were supplemented with 1% penicillin-streptomycin (15140122, ThermoFisher Scientific, USA) to prevent cell contamination during culture. Basal MSCs were stained with either Hoechst (Hoechst 33342, H3570, ThermoFisher Scientific, USA) for 10 minutes or cell tracker (C2102, CellTracker™ green BODIPY™ dye, ThermoFisher Scientific, USA) for 15 minutes. In all cases, adhesive cells were detached using Trypsin/EDTA (CC3232, Lonza) at 37° C. for 10 minutes. The size of trypsinized cells was measured by the analytical tool provided in the Olympus cellSens Dimension software.

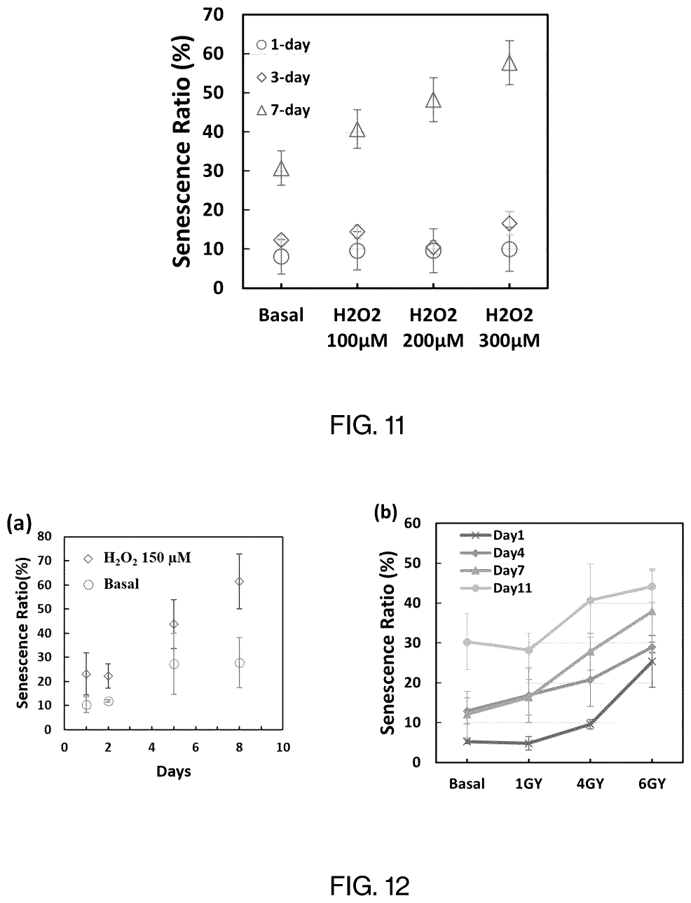

Senescent cell model and staining. hMSCs or mMSCs senescence was induced chemically using hydrogen peroxide or using X-ray radiation. The medium containing hydrogen peroxide (H₂O₂) was prepared by diluting 30% H₂O₂ solution (H1009, Sigma, USA) with MSCs culture medium to desired concentrations. For the H₂O₂ treatment experiments, the MSCs were cultured in the medium containing H₂O₂ at 37° C. for 3 hours. After that, the MSCs were washed with 1×DPBS 2 times and cultured in the fresh media for a variable number of days before analysis. For X-ray radiation treatment, MSCs were placed on a rotating table and exposed to 1 gray (Gy), 4 Gy, or 6 Gy radiation. After treatment, cells were cultured for a variable number of days before analysis. The senescent cells were identified by the senescence detection kit (K320, BioVision, USA), which targets senescence-associated β-galactosidase (SA-β-gal) activity, following the manufacturer's provided protocol. The senescence progression in response to H₂O₂ concentration or X-ray radiation dosage over time is shown in FIG. 11 (hMSCs) and S4 (mMSCs). For the experiments using the mixture of basal and H₂O₂-treated hMSCs, the H₂O₂-treated hMSCs were stained with cell tracker (C34551, CellTracker™ orange CMRA Dye™ dye, ThermoFisher Scientific, USA), while basal hMSCs were stained with Hoechst for 10 minutes to differentiate two cell populations.

Cellular senescence induction of human MSCs. Hydrogen peroxide (H₂O₂) was used to induce cellular senescence of human MSCs. We tested three H₂O₂ concentrations: 100, 200, and 300 μM. Concentrated H₂O₂ (100×) was prepared and was added to hMSC culture media using a 1 to 100 dilution, after which, hMSCs were cultured in H₂O₂ contained medium in a 37° C. incubator for three hours. After H₂O₂ treatment, the H₂O₂ contained medium was withdrawn and the culture wells were washed with DPBS two times. Fresh culture medium was then added to cells for 1, 4, and 7 days post-culture. hMSCs were detached using Trypsin/EDTA, then labeled with a senescence detection kit in a 37° C. incubator overnight following the manufacturer's recommended protocol (K320, BioVision, USA). The senescence kit targets senescence-associated β-galactosidase (SA-β-gal) activity within cells. The SA-β gal⁺ cells and total cells were counted to estimate the senescence cell ratio. The senescence ratios of hMSCs under various conditions are shown in FIG. 11. For basal and H₂O₂ treated hMSCs, the senescence ratio significantly increased at 7 days after treatment in an H₂O₂ dose-dependent manner. hMSCs were treated with 300 μM H₂O₂, and cells were analyzed 7 days after treatment to obtain a ˜50% senescence ratio. For the experiment to mix basal hMSCs with H₂O₂ treated hMSCs, the basal hMSCs followed the same culture protocol, but without H₂O₂ treatment.

Cellular senescence induction of mouse MSCs. Cellular senescence of mouse MSCs was induced by hydrogen peroxide (150 μM, H₂O₂) or X-ray irradiation (1, 4, 6 gray (Gy)). A lower H₂O₂ concentration was used to induce senescence in mMSCs since they are more vulnerable to H₂O₂ induced senescence compared to hMSCs. For X-ray induced senescence, mMSCs were exposed to 0, 1, 4, and 6 Gy X-ray irradiation using a Precision X-ray Inc X-RAD320 X-ray machine operated at 300 kV, 10 mA (dose rate of 1.3 Gy/min). After radiation exposure, the culture medium was replaced with a fresh culture medium and cells were cultured for an additional 1, 4, 7,or 11 days before analysis. At each timepoint, mMSCs were labeled with a senescence detection kit in a 37° C. incubator overnight. The senescence cell ratio was calculated by dividing the SA-β gal⁺ cells over the total number of cells counted. The senescence cell ratios of mMSCs after treatment with H₂O₂ or X-ray irradiation are shown in FIG. 12. In H₂O₂ treated mMSCs, the senescence ratio was higher compared to those without H₂O₂ treatment at each timepoint including 24 hours after treatment (panel (a) of FIG. 12) In both treated and untreated cells, the senescence ratio increased over time. In X-ray irradiation treated mMSCs the senescence ratios increased with the X-ray dosage and time as shown in panel (b) of FIG. 12. The induction of senescence by 6 Gy X-rays was already significant 24 hours after exposure. Basal mMSCs and mMSCs treated with H₂O₂ or 6 Gy at 24 hours after treatment were collected for conducting the separation experiment shown in FIG. 4.

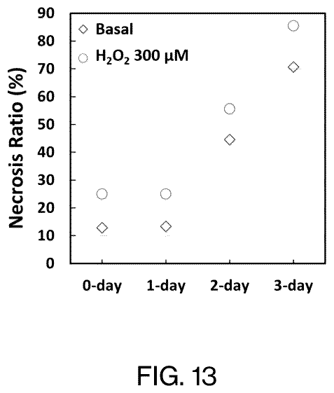

Necrotic cell model and staining. To induce cell necrosis, the detached cells after culture were stored in a 4° C. refrigerator (GDM-23-SCI-TSL01, VWR, USA) for a variable number of days. The ratio of necrotic cells increased with the storage time. The apoptotic/necrotic cells were identified by the apoptosis kit (K201, BioVision, USA), using annexin V-FITC and SYTOX™ green dye, by following the manufacturer's provided protocol. The annexin V-FITC targets phospholipid phosphatidylserine on the surface membrane due to apoptosis, while SYTOX™ green dye targeting nucleic acid in the necrotic cells showed a higher level of green fluorescence. The fraction of necrotic cells as a function of the storage time is shown in FIG. 13.

Necrosis induction of human MSCs. To acquire a human MSC population with a ˜50% necrosis ratio, after hMSC treated with or without H₂O₂ and post-cultured for 7 days, collected hMSCs were stored at 4° C. for 1, 2, or 3 days to induce necrosis. hMSCs were stained using an apoptosis kit (K201, BioVision, USA) to determine the necrotic ratio at different storage times. To isolate necrotic cells from a healthy cell population, necrotic hMSCs we counted based on bright green fluorescence. In general, the necrosis ratio increased with storage time. Two days after being stored at 4° C., the necrosis ratio reached ˜50%, and basal and H₂O₂ treated hMSC were mixed for conducting separation experiment as shown in FIGS. 9 and 10.

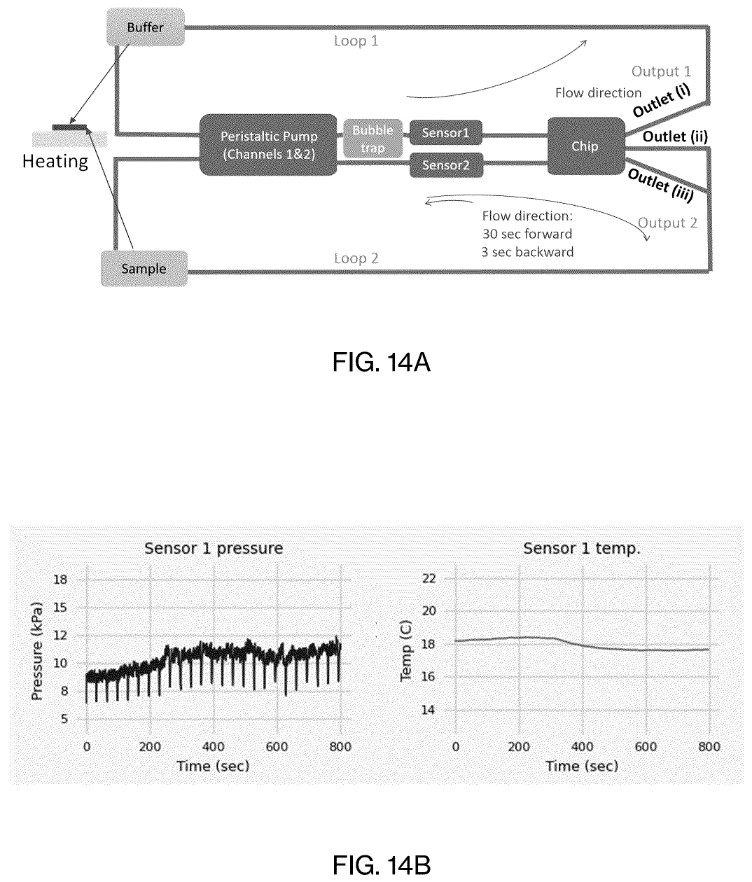

Cycling fluidic system. To investigate the feasibility of in vivo cell separation, a close loop fluidic system as described herein was constructed, where buffer inlet and the outlet (i) connect to the tube containing the buffer as Loop 1, while sample input, outlet (ii), and outlet (iii) connect to the tube containing the sample as Loop 2. The illustration of the close loop fluidic system is shown in FIG. 14A. Both sample and buffer were kept at 37° C. by a heating instrument to mimic mouse or human body temperature. Both Loops were equipped with a sensor (Sensor1 and Sensor2, LabSmith, Livermore, CA) before the inlets to the device to measure pressure (kPa) and temperature (° C.) in real-time. A bubble trap was added before Sensor1 to prevent bubbles from entering into the device. To further reduce the chance of cell clogging during operation, a proprietary Python script was written to control the flow direction applied by the peristaltic pump (Ismatec Reglo, Cole-Parmer, Vernon Hills, IL). Sensors were electronically connected to 4AM01 uProcess (LabSmith, Livermore, CA) analog sensor manifold (ARM cortex Raspberry Pi-3b (Raspberry Pi, Cambridge, U.K)) for signal detection. LabSmith sensors were scripted using python library-LabSmith-uProcess 1.5.7. The pressure (kPa) and temperature (C) reading of Sensor1 were plotted as shown in FIG. 14B.

Mouse model and staining. Male C57BL/6 mice at ten weeks of age were exposed to 6.5 Gy X-ray (n=4) or sham control (n=4), using a Precision X-ray Inc X-RAD320 320 kVp X-ray machine (Precision X-ray Inc., North Branford, CT), operated at 300 kV, 10 mA (dose rate of 1.3 Gy/min). Mice were returned to their cage and left undisturbed for 6 days, then euthanized before collection of their bone marrow. Bone marrow samples were collected from the hind-leg femurs by flushing the contents of the marrow with approximately 5 mL of 1×PBS supplemented with 0.5% FBS and 8 μM EDTA. The bone marrow cell suspension was filtered through a 70 μm nylon filter and further diluted with 1× DPBS buffer to a total volume of 10 mL. For samples that could not be processed on the same day of collection, Tirofiban (30 μg/mL; SML0246-10 MG, Sigma, USA) was added to the bone marrow sample before storage at 4° C. to alleviate cold-induced platelet aggregation. The MSCs and Hematopoietic Stem Cells (HMCs) were identified by Hoechst, Stem Cells Antigen-1 antibody (anti-sca-1, 130-116-490), Platelet-derived Growth Factor Receptor-a antibody (anti-PDGFRa or anti-CD140a, 130-102-502), and c-Kit antibody (anti-c-Kit, or anti-CD117, 130-122-948). Anti-sca-1, anti-CD140a, and anti-CD117 antibodies were purchased from Miltenyi Biotech, USA. The procedure of cell staining and the gallery of cell images are summarized as described above and elsewhere herein. All bone marrow samples were filtered with Pre-Separation Filters (130-095-823, Miltenyi Biotech) to remove large debris before loading to the device. For all experiments with the mouse bone marrow, the samples were treated with RBC lysis buffer (420301, BioLegend, USA) at room temperature for 30 minutes to reduce the number of red blood cells before staining or cell counting.

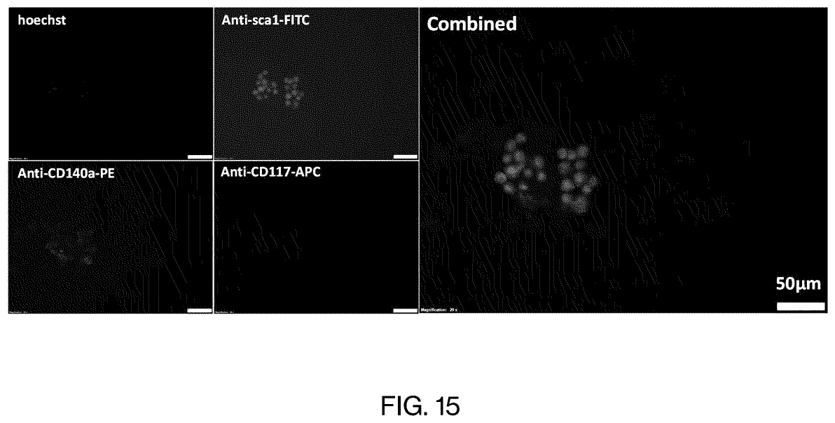

Staining of mouse MSC cells. Mouse MSCs (GIBCO® Mouse (C57BL/6) Mesenchymal Stem cells) were stained by Hoechst, anti-scal-FITC, anti-CD140a-PE, and anti-CD-117-APC following the manufacturer's protocol and imaged in four separated channels, i.e., blue, green, orange, and far-red channels. The concentration of Hoechst, anti-scal-FITC, anti-CD140a-PE, and anti-CD-117-APC was 2, 3, 5, 3 μL in 100 μL DPBS containing 4.5% BSA (A3059, Sigma, USA). All staining was performed on a membrane (WHA110412, Nuclepore™ Track-Etched Membranes, Millipore, USA) at 4° C. for 20 minutes. The membrane was pre-incubated with 4.5% BSA for 10 minutes. After staining, the membrane was mounted on a glass slide using ProLong™ Gold antifade mountant (P36941, ThermoFisher Scientific, USA) then covered by a cover glass before taking images on the microscope. The fluorescence images of 4 separate channels and the combined image are shown in FIG. 15. The mMSCs expressed scal and CD140a antigens but not CD117 antigens. Per the company's manual, the C57BL/6 strain mMSC is 70% positive to scal and is negative to CD117 (<5%). mMSCs from C57BL/6 mice may contain both scal CD140a and scal CD140a populations. Some sca1⁺ mMSCs showed higher expression of CD140a while others show less expression in CD140a. The exposure time for Hoechst, FITC, PE, and APC was 10-30, 200-300, 200-300, and 200-300 ms, respectively.

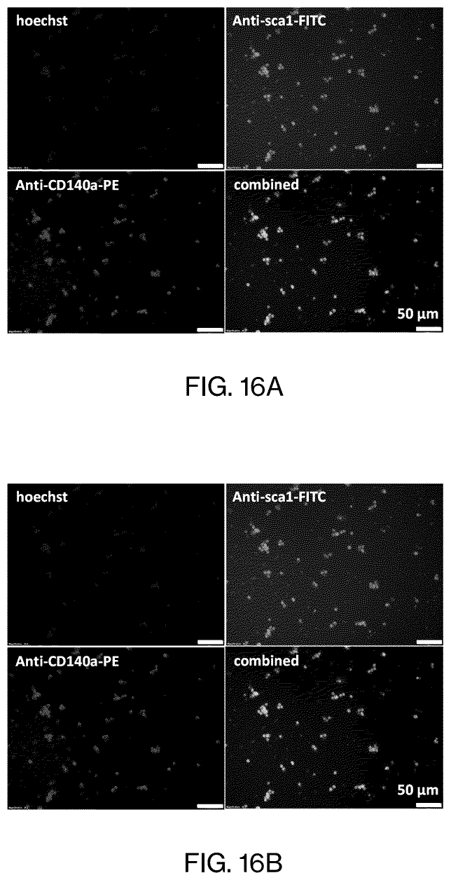

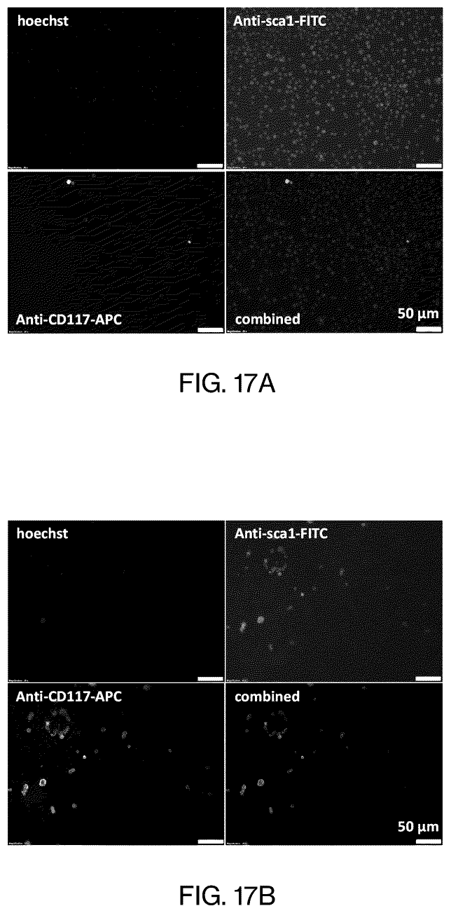

Staining of cells from mouse bone marrow samples and mouse MSC cell lines. For mouse bone marrow cell staining, samples were pre-filtered to remove large debris, after which cells were collected by centrifugation. The cell samples were resuspended and fixed using 4% formaldehyde at 4° C. for 20 minutes. The staining procedure of bone marrow samples is similar to the staining of the mMSC cells as described elsewhere herein. The images of Hoechst, anti-scal, anti-CD140a channels, and combined channel images of sham irradiated mouse MSCs and 6.5 Gy irradiated mouse MSCs are shown in FIGS. 16A and 16B. The images of Hoechst, anti-scal, anti-CD117 channels, and combined channel images of sham mice HSCs and 6.5 Gy irradiated mouse HSCs are shown in FIGS. 17A and 17B.

Fabrication and Characterization of the 2-stage Device. An illustration of a 2-stage device and system in accordance with the present disclosure is shown in the top left in FIG. 8A, where the enlarged illustration of filter features on the x-y plane is highlighted by a blue box. The main features include θ₁, θ₂, and Id which are 4°, 30°, and 8 μm, respectively, and the sd, shortest distance, is ˜4 μm. The θ₁ is the inclination angle of the pillar array relative to the main fluidic flow. The θ₂ is the smaller angle in the single unit of pillar array. The ld is the longest inter-pillar spacing, while sd is the shortest inter-pillar spacing. The diameter of the particle or cell that is larger than 8 μm (cutoff particle 2R, where R is the radius) is expected to roll along with the filter feature and will not be captured in the space between two filter features. The average cell size of human mesenchymal stem cells (hMSCs) and mouse mesenchymal stem cells (mMSCs) is larger than 8 μm, so they are least likely to be captured at the gaps between the filter features. The fabrication process of the device is summarized in the right panel of FIG. 8A. The images of a 2-stage device (left) and 1-stage device (right) in accordance with the present disclosure are shown in FIG. 8B. In the 2-stage device, the larger particles/cells may roll over the filter array at the first stage then flow to the top branch at the second stage for further separation.

FIG. 8C shows the cross-section of the device. The cross-sections of the device of various channel heights and z-gaps shown in FIG. 8C include: (1) for hMSCs separation, (2) for mMSC or beads separation, and (3) for in-vitro bone marrow-derived mice samples separation. The z-gap and the channel height are marked, and their dimensions are listed in the table. By adjusting the z-gap, the device can separate cells of different sizes.

The separation efficiency for beads of 20 μm and 15 μm, and basal mMSCs, using 2-stage and 1-stage device is summarized in FIG. 8D. Both the 2-stage and 1-stage devices could sort 15 μm or 20 μm beads at an efficiency of >80% to the outlet (i). However, compared to a 1-stage device (14.3%), much fewer basal mMSCs were collected at the outlet (i) when using a 2-stage device (2.8%). The flow controlled by a peristaltic pump generated a sinusoidal flow stream within the chip because of the pressure pulsation in the fluidic system. The sinusoidal flow stream caused the leaking of some small basal mMSCs to the outlet (i). By using a 2-stage device, most small basal mMSCs leaked in the 1^{st} stage flowed through the top branch to the outlet (ii), not the outlet (i). Only larger senescent cells were expected to remove from the cell solution and collected at the outlet (i). A >80% recovery rates of beads and mMSCs was observed, and only a few beads/cells were found clogged in the filter array area when the loading concentration of bead/cell is >10⁴/mL.

Separation of hMSCs by the 2-stage Device. The 2-stage device described above was used to separate hMSCs based on their cell fates. Senescence of hMSCs can be induced by H₂O₂. The percentage of senescent cells increased with the post-culture time for both untreated and H₂O₂ treated hMSCs. The percentage of senescent hMSCs after treatment with 300 μM H₂O₂ increased from 9.9%, 16.5%, to 57.7% for 1, 3, and 7 days post-treatment (FIG. 11). On the other hand, the senescence percentage of basal hMSCs increased from 8%, 12.3%, to 30.7% after 1, 3, and 7 days of culture without H₂O₂ treatment (FIG. 11). The percentage of cellular senescence was significantly different between untreated and H₂O₂ treated cells 7 days post-treatment (p=0.0038). On the other hand, the necrotic cell percentage of H₂O₂ (300 μM) treated hMSCs increased from 25% to 56% while basal hMSCs cells from 12 to 44% when stored at a 4° C. refrigerator for 2 days (FIG. 13).

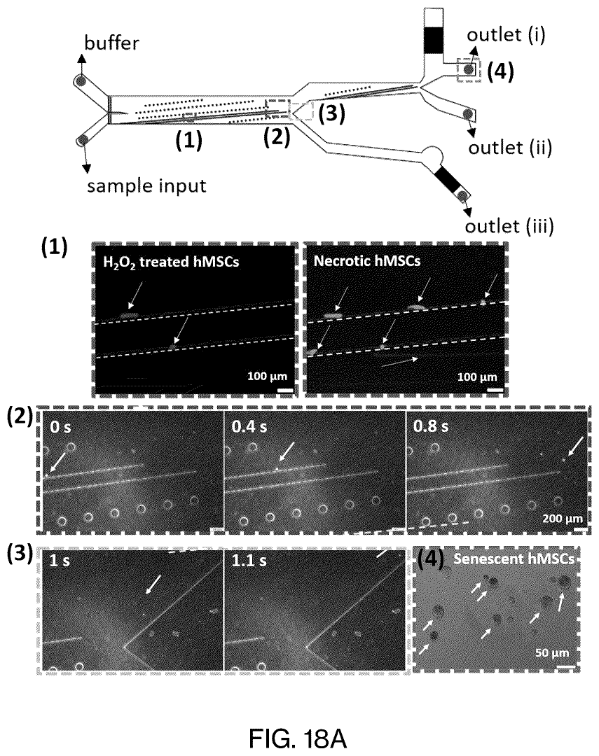

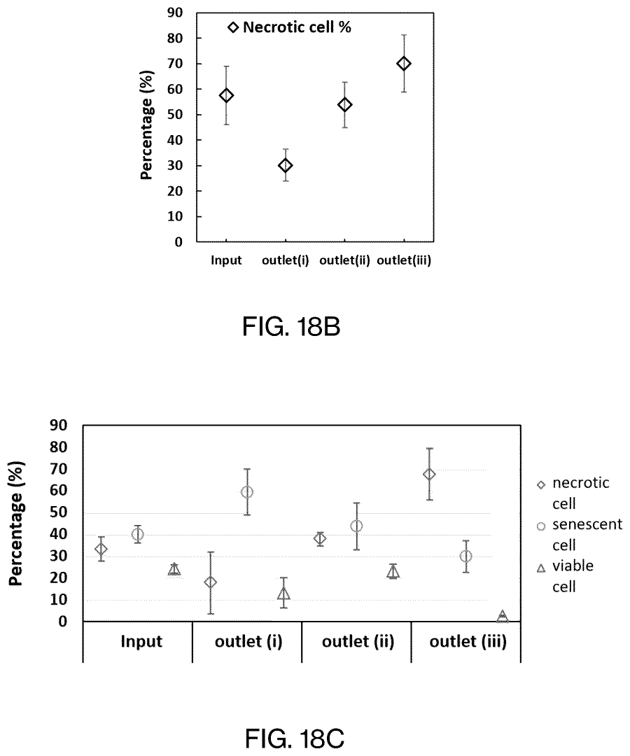

The stored H₂O₂ treated hMSCs were prepared at a cell density of ˜10⁴/mL as the sample input. The device with a z-gap of 12.1+/−2 μm and a channel depth of 54.9+/−1 μm was used for hMSC separation. The represented images of hMSCs running on the 2-stage device in the highlighted area (1) were shown in FIG. 18A. Most H₂O₂ treated hMSCs were rolling on the filter array (dot line in FIG. 18A), while necrotic hMSCs were able to pass through the filter array. It was observed at the area (3) in the top panel of FIG. 18A that many more green cells flowed to the lower branch than the upper branch. The timelapse images of the flow trajectory in 1.1 secs for an hMSC cell in the area (2) to (3) are shown in the bottom panel of FIG. 18A. The cell flowed on the filter array then flowed to the upper branch of the device. Around 50-70% hMSCs acquired from the outlet (i) were senescent cells, as confirmed by the SA-β-gal⁺ staining after collection from the outlet (panel (4) of FIG. 18A).

H₂O₂ treated hMSCs containing about 60% necrotic cells were applied to the 2-stage device. The percentage of necrotic hMSCs acquired from the outlet (i), outlet (ii), and outlet (iii) was 30.2+/−6.3%, 53.8+/−8.9%, and 70.1+/−11.2%, respectively (FIG. 18B). The necrotic cell percentage in the solution collected at the outlet (i) was the lowest while highest at the outlet (iii), and cell percentage collected at the outlet (ii) was close to that in the sample input.

The separation efficiency of device for separating senescent and necrotic hMSCs was evaluated. The basal hMSCs were mixed with H₂O₂ treated hMSCs to generate a mixture of ˜40% senescent and ˜35% necrotic hMSCs as the sample input. Applying this sample to the 2-stage device, the necrotic, senescent, and viable hMSC percentage at the outlet (i) was 18.5%, 60%, and 12.5%, at the outlet (ii) was 38.5%, 44%, and 23%, and at the outlet (iii) was 68.5%, 30%, and 4.5% (FIG. 18C). The reagent in the senescence detection kit does not label the necrotic cells because the beta-galactosidase enzymatic activity, which catalyzes the hydrolysis of β-galactosides into monosaccharides, is lost in the necrotic cells. Senescent cells were observed to be the dominant population at the outlet (i), while necrotic cells were the dominant population at the outlet (iii). The percentage of the viable cell population was lower in both outlet (i) and (iii), compared to the sample input. Senescent hMSCs tended to flow to the outlet (i), while necrotic hMSCs tended to flow to the outlet (iii). The percentages of the three cell populations collected at the outlet (ii) were similar to that of the sample input. By adjusting the z-gap of the device, only smaller necrotic cells were allowed to flow through the pillar array to the outlet (ii) or (iii) with minimum deformation.

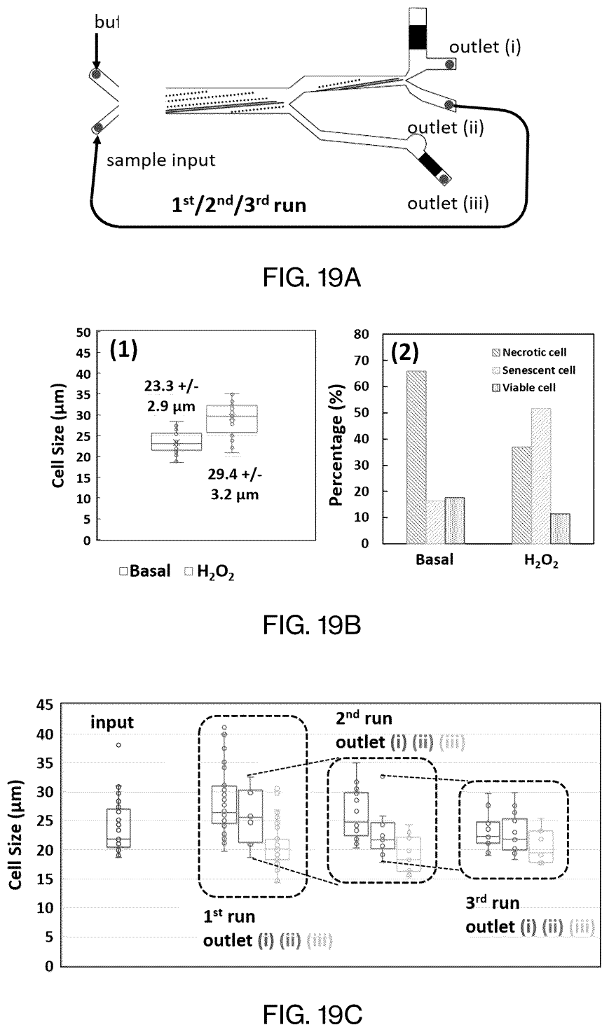

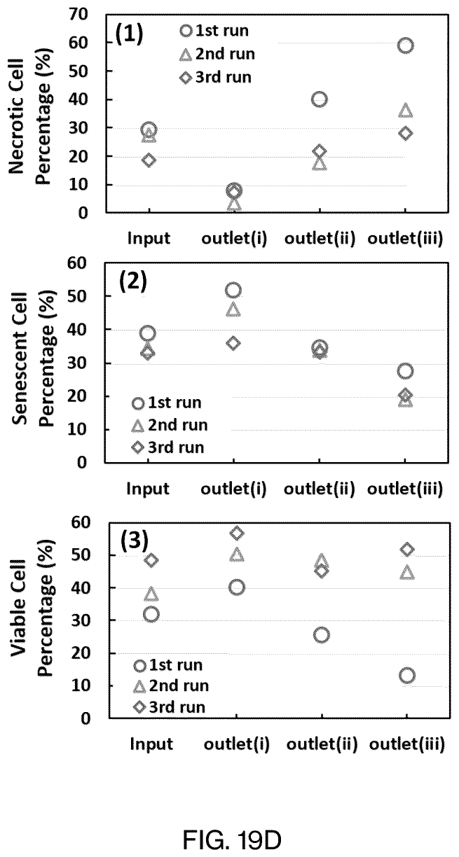

Since a 2-stage device could separate hMSC based on their fates, with enrichment of senescent cells at the outlet (i), necrotic cells at the outlet (iii), and viable cells at the outlet (ii), its potential for high-throughput, high-efficiency viable cell enrichment, through a sequential separation procedure and system was further investigated (FIG. 19A). The cells collected at the outlet (ii) were reintroduced to sample inlet, and the procedure was repeated two more times. At each run, the cell size, the percentages of necrotic, senescent, and viable cells were measured. The basal and H₂O₂ (300 μM) treated hMSCs were mixed as the sample input, where the size of the basal and H₂O₂ treated hMSCs was 23.3+/−2.9 μm and 29.4+/−3.2 μm, respectively (left panel of FIG. 19B). Both basal and H₂O₂ treated hMSCs were cultured for 7 days and stored at a 4° C. refrigerator for another 2 days. The percentage of necrotic, senescent, and viable cells of basal was 65.9%, 16.5%, and 17.6%, respectively, while they were 37%, 51.7%, and 11.3%, respectively for H₂O₂ treated hMSCs (right panel of FIG. 19B).

The basal and H₂O₂ treated hMSCs were mixed to have the sample input with ˜30% necrotic cells, ˜40% senescent cells, and ˜30% viable cells. The cell size distribution in the sample input, from the outlet (i)-(iii) after 1^{st} run of separation, from the outlet (i)-(iii) after 2^{nd} run of separation, and outlet (i)-(iii) after 3^{rd} run of separation is shown in FIG. 19C. The average size of the cells in the sample input was 23.8+/−4.3 μm. The average size of cells from the outlet (i), (ii), and (iii) after 1^{st} run of separation was 27.8+/−5.1 μm, 25.5+/−4.3 μm, and 20.5+/−3.7 μm, respectively. The size of cells collected at the outlet (iii) was ˜5 μm and ˜7.3 μm smaller than those collected at the outlet (ii) and outlet (i), respectively. The difference between cell size from the outlet (iii) compared to the outlet (i) or between outlet (iii) and outlet (ii) was statistically significant with a p-value<0.001 and 0.006, respectively. After the 2^{nd} run of separation by device, the average size of cells from the outlet (i), (ii), and (iii) was 26+/−3.9 μm, 22.6+/−4 μm, and 18.9+/−2.9 μm, respectively. The size of cells collected at the outlet (iii) was ˜3.7 μm and ˜7.1 μm smaller than those collected at the outlet (ii) and outlet (i), respectively. The difference between cell size from the outlet (iii) and outlet (i) and between outlet (iii) and outlet (ii) was statistically significant with a p-value<0.001 and 0.024, respectively. After the 3^{rd} run of separation, the size of cells from the outlet (i), (ii), and (iii) was 23.1+/−3 μm, 22.7+/−3.3 μm, and 20.4+/−2.6 μm, respectively. The size of cells collected at the outlet (iii) was ˜2.3 μm and ˜2.7 μm smaller than those collected at the outlet (ii) and outlet (i), respectively. The difference between cell size from the outlet (iii) and outlet (i) and between outlet (iii) and outlet (ii) was statistically significant with a p-value of 0.035 and 0.07, respectively. However, the size difference between outlet (ii) and outlet (iii) was not statistically different (p>0.05). In summary, the total cell size distribution became smaller after each run of separation on a device (FIG. 19C) as larger (senescent) and smaller (necrotic) cells were gradually removed at each run with the enrichment of viable cells.

The enrichment of each cell fate at the three outlets for the three cycles was analyzed (FIG. 19D). For all three runs, the cells collected from the outlet (i) had the lowest necrotic cell percentage while cells from the outlet (iii) had the highest percentage. After three cycles, the percentage of necrotic cells in the sample input decreased from 29% to 19%, and from 59% to 28% in the outlet (iii), suggesting that necrotic cells were removed gradually at each run. On the other hand, the cells collected from the outlet (i) had the highest senescent percentage while outlet (iii) had the lowest. After three cycles, the percentage of senescent cells in the sample input decreased from 39% to 33%, and from 52% to 36% in the outlet (i), suggesting that senescent cells were removed at each run. On the other hand, the percentage of viable cells in the sample input increased from 32% after the first cycle to 48% after the third cycle as shown in FIG. 19D. The percentage of viable cells increased in each outlet after the 2^{nd} and 3^{rd} runs. Overall, through three sequential cycles of separation by the device, an enrichment in viable cells (from 32% to 48%) was observed, and the size distribution range became more uniform. Therefore, the 2-stage device could increase the enrichment of viable cells by increasing the number of separation cycles.

Separation of mMSCs by the 2-stage Device. For separating the mMSCs, the z-gap of the 2-stage device was adjusted to 8.2+/−1.5 μm with the channel depth of 40.1+/−1.6 μm as listed in FIG. 1(c). The average size (cultured for 1 day) of mMSC was 13.3+/−1.1 μm. We used H₂O₂ or X-ray radiation to induce cellular senescence of mMSCs as shown in Figure S4. The mMSCs were less resistant to the H₂O₂ compared to hMSCs, and a lower concentration of 150 μM was on mMSCs for three hours. On the other hand, a dosage of 6 Gy was used for X-ray radiation. After culturing for 1 day, the size of mMSCs treated with H₂O₂ or X-ray radiation was 16.8+/−2.5 μm or 17.1+/−2.7 μm, with an increase of 3.5 or 3.8 μm compared to basal mMSCs, respectively.

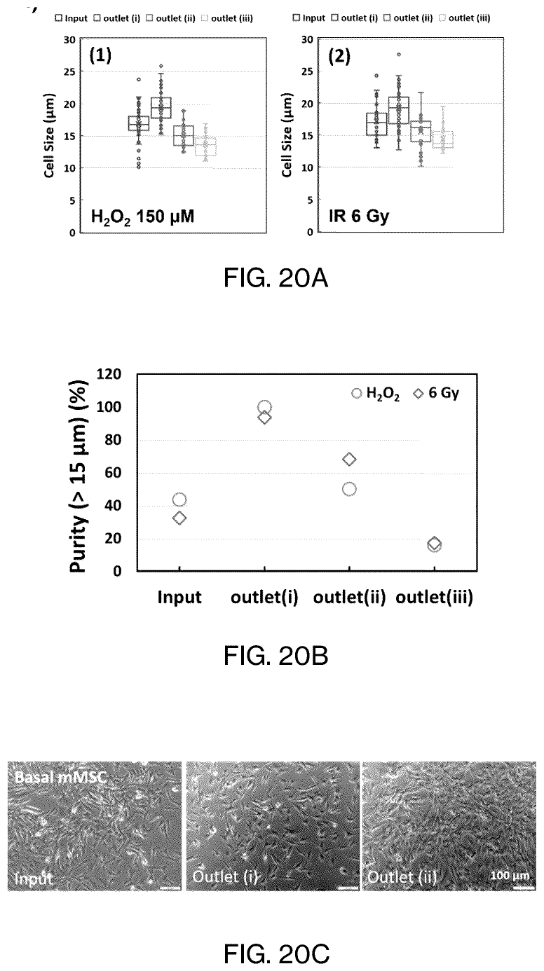

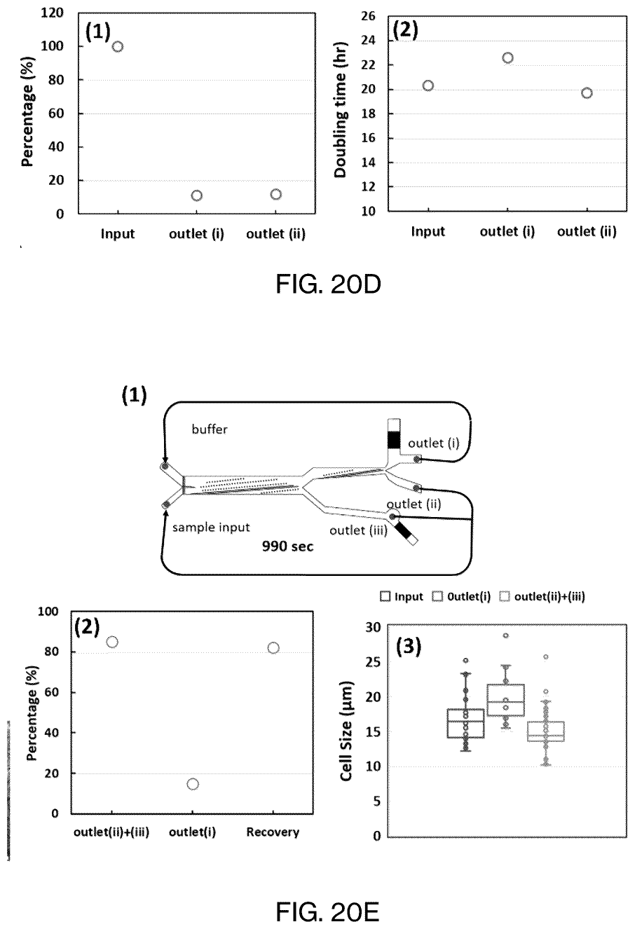

The H₂O₂ treated mMSCs were separated by the device (FIGS. 20A-13E). The average size of mMSCs collected at the outlet (i), (ii), and (iii) was 19.3+/−2.5 μm, 15.1+/−1.9 μm, and 13.6+/−1.7 μm, respectively. The average size of mMSCs collected at the outlet (iii) was ˜1.5 μm and ˜5.7 μm smaller than those collected at the outlet (ii) and outlet (i), respectively. The difference in cell size between outlet (iii) and outlet (i) was statistically different (p-value<0.001). In the case of mMSCs treated with 6 Gy radiation, the size of cells from the outlet (i), (ii), and (iii) was 19.2+/−3.2 μm, 15.5+/−2.6 μm, and 14.4+/−1.9 μm, respectively. The size of cells collected at the outlet (iii) was ˜1.1 μm and ˜4.8 μm smaller than those collected at the outlet (ii) and outlet (i) (p-value<0.001). Considering that the average cell size in the input solution was similar for these two cell inputs, the separation performance based on the cell size was very consistent.

The purity of larger mMSC (>15 μm) at each outlet was analyzed as shown in FIG. 20B. The threshold of 15 μm was used to estimate the capture rate and purity since the size change of senescent cells was dynamic. The purity rate was defined as (target cells collected in outlet/total cells collected in outlet)×100%. The purity of cells >15 μm was ˜5%, 43.5%, 32.5% for the basal, H₂O₂ treated, and X-ray radiated mMSCs, respectively. The population of >15 μm cells significantly increased when mMSCs were treated with H₂O₂ or X-ray radiation. For H₂O₂ treated mMSCs, the purity of mMSCs >15 μm at outlet (i), outlet (ii), and outlet (iii) was 100%, 50%, and 16%, respectively. For X-ray radiated mMSCs, the purity of mMSC >15 μm at outlet (i), outlet (ii), and outlet (iii) was 93.8%, 68.2%, and 17.1%, respectively. For both experiments, the purity of mMSCs >15 μm collected in the outlet (i) >90% suggested the ability of device to enrich >15 μm mMSCs. The capture rate of mMSCs >15 μm through the device. The capture rate was defined as: (target cells collected in the outlet/target cell collected in inlet)×100%. Combining outlet (i) and (ii), the capture rate of mMSCs >15 μm at outlet (i) and (ii) for H₂O₂ treated and X-ray radiated mMSCs was 80% and 60%, respectively.

Basal mMSCs were also separated through the 2-stage device under sterile conditions, and the growth rate of cells collected from the sample input, outlet (i), and outlet (ii), respectively, was studies. The cell images after culture for 7 days are shown in FIG. 20B. The cell viability of cells collected from all outlets (over cell input in the inlet) was >95%. The cells collected at outlets (i) and (ii) were healthy and could grow and proliferate. The percentages of cells in the sample input, outlet (i), and outlet (ii) are shown in FIG. 20D. As expected, most of the basal mMSCs flow to the outlet (iii). The doubling time of mMSCs collected from input, outlet (i), and outlet (iii) was found to be 20, 22.6, and 19.7 hours, respectively. The mMSCs collected at the outlet (i) grew more slowly compared to the sample input and those collected at the outlet (ii). The slower proliferation of cells suggested a higher population of senescent cells collected at the outlet (i). This result shows that the 2-stage device can separate senescent cells from the viable cell population for basal mMSCs.

Next, an automatic cyclic fluidic system including sensors, valves, and bubble trappers to circulate fluidics in two closed loops, was discussed. One loop connected the buffer inlet with the outlet (i), and another loop connected the sample input with outlets (ii) and (iii), as shown in FIG. 20E. The pump and sensors were controlled using a proprietary python script. Using the closed-loop system for separation of the mixture of basal and H₂O₂ treated mMSCs on device, the average percentages of cells in the outlet (i) or outlet (ii)+(iii) were 15.1+/−7.6% or 84.9+/−7.6%, respectively (FIG. 20E, bottom left). The recovery rate after a 990-second operation was 82.2%. The average size of mMSCs in the sample input or collected at the outlet (i), (ii)+(iii) after the 990-second operation was 16.7+/−3.2 μm, 19.9+/−1.9 μm, and 15.1+/−2.9 μm, respectively (FIG. 20E, bottom right). The average size of mMSCs collected at the outlet (ii)+(iii) was ˜4.8 μm smaller than that at the outlet (i). Therefore, larger mMSCs in the buffer-outlet (i) loop were separated through the automatic cyclic fluidic system.

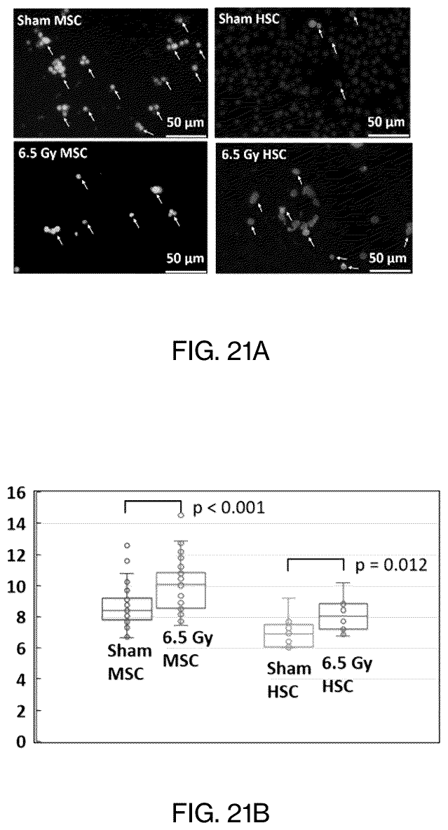

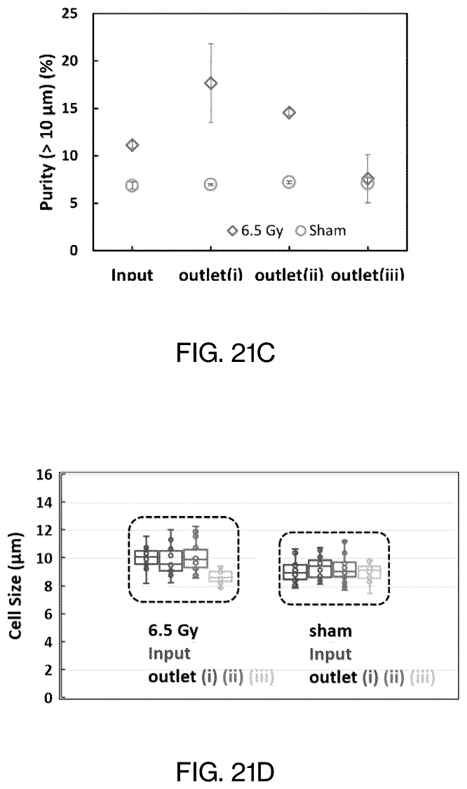

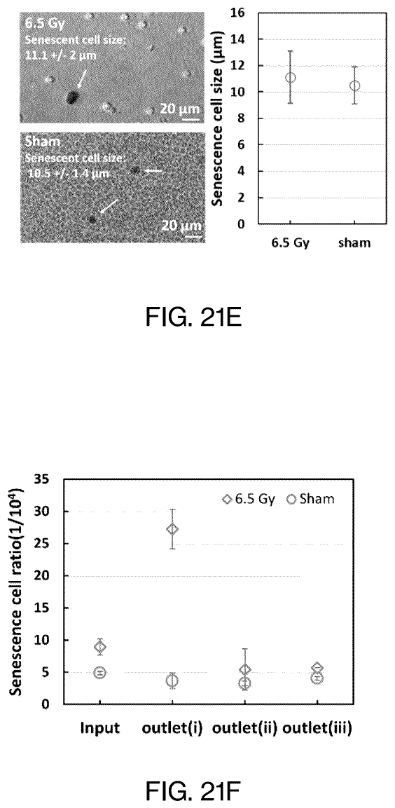

Separation of Mouse Bone Marrow Samples using the 2-stage Device. To investigate the size distribution of MSCs and Hematopoietic stem cells (HSC) in mouse bone marrow, bone marrow cells were collected and stained by Hoechst, anti-sca-1, anti-CD140a, and anti-CD117. The Hoechst stains the nucleus of MSCs and HSCs. The anti-scal conjugated with Vio® Bright FITC, anti-CD140a conjugated with PE, and anti-CD117 conjugated with APC were visualized in the green, red, and white color channel, respectively (FIG. 21A). Sca1⁺CD140a⁺ double-positive cells are considered MSC while sca1⁺CD117⁺ double-positive cells are considered HSC. The mMSC cell line is anti-sca1⁺CD140a⁺ double-positive and CD117⁻ negative.

The size distribution of bone marrow-derived MSC and HSC found in sham or 6.5 Gy irradiated mice are shown in FIG. 21B. Bone marrow-derived MSC was larger than HSC independent of X-ray exposure. The average size of bone marrow-derived MSCs in 6.5 Gy irradiated mice (10+/−1.6 μm) was larger than that in sham irradiated mice (8.7+/−1.4 μm) (p-value<0.001). The average size of bone marrow-derived HSCs in 6.5 Gy irradiated mice (8+/−1 μm) was also larger than that in sham control mice (7+/−1 μm) (p=0.012).

Compared to the size of mMSC cell lines in FIGS. 20A-20E, which was 13.3+/−1.1 μm, the size of MSCs from mice samples was 3-4.5 μm smaller on average, consistent with the fact that self-renewing cells may be smaller in diameter. To separate the mouse bone marrow-derived cells, the z-gap of the 2-stage device was further reduced to 5.1+/−0.5 μm and the channel depth of the chip was 37.1+/−2.6 μm. To prevent coagulation within the chip, 1.5 mg/mL K₂-EDTA was added to mMSC culture media. Bone marrow samples, which contained K₂-EDTA already, from either sham or 6.5 Gy irradiated mice, were allowed to flow through the 2-stage device. The purity of cells >10 μm at sample input, outlet (i), outlet (ii), and outlet (iii) for sham irradiated mice (red circle) or X-ray irradiated mice (blue diamond) is shown in FIG. 21C. The purity of cells >10 μm in the sham and 6.5 Gy irradiated mouse bone marrow samples was 6.9+/−0.4% and 11.2+/−0.4%. Around 4.3% more >10 μm cells were observed in samples from 6.5 Gy radiated mice. Excluding the red blood cells in the sample, the percentage of cells >10 μm in mice bone marrow samples from the sham and 6.5 Gy irradiated mice was 21.2+/−0.5% and 36.6+/−2% (only count cells >6 μm), respectively, showing a significant increase of >10 μm cells in the irradiated mice bone marrow. The purity of cells >10 μm at outlet (i), outlet (ii), and outlet (iii) for X-ray radiated mice was 17.7+/−4.2%, 14.6+/−0.4%, and 7.6+/−2.5%, respectively. The difference between outlet (i) and outlet (iii), and between outlet (ii) and outlet (iii) was statistically significant (p-value<0.001). On the other hand, the purity of cells >10 μm at outlet (i), outlet (ii), and outlet (iii) for sham irradiated mice was 7+/−0.1%, 7.2+/−0.1%, and 7.1+/−0.6%, respectively. There was no significant difference in purity of cells >10 μm between the three outlets, and all percentages were close to that in the sample input. There was a significant increase in the purity of cells >10 μm at the outlet (i) or outlet (ii) between 6.5 Gy irradiated mouse bone marrow samples and sham irradiated control samples.

As shown in FIG. 21D, the average cell size at the input, outlet (i), outlet (ii), and outlet (iii) for 6.5 Gy irradiated mice was 10.1, 9.9, 10.1, and 8.9 μm, while the average cell size at input, outlet (i), outlet (ii), and outlet (iii) for sham irradiated mice was 9.2, 9.4, 9.2, and 9.1 μm. For irradiated mouse-derived bone marrow samples, the average size of cells collected at the outlet (iii) was smaller than those at the outlet (i) or (ii). On the other hand, there was no significant difference in the average size of cells collected at each outlet in bone marrow samples from sham irradiated mice.

SA-β-gal⁺ cells in the 6.5 Gy or sham irradiated mouse bone marrow samples at each outlet were identified. Representative SA-β-gal⁺ images are shown in FIG. 21E. The average bone marrow-derived senescence cell size in sham or 6.5 Gy irradiated mice was 10.5+/−1.4 μm or 11.1+/−2 μm, respectively (p=0.12). After exposure to 6.5 Gy X-rays, mice were returned to their cage and left undisturbed for 6 days before euthanasia. The number of senescent cells in the sample input and solution collected from the outlet (i), outlet (ii), and outlet (iii) was then counted. The senescent cell concentration in 6.5 Gy irradiated mice in the input, outlet (i), outlet (ii), and outlet (iii), was 8.6, 26.2, 5.2, and 5.4 per 1×10⁴ cells (population of cell size >10 μm), respectively (FIG. 21F). The capture rate of senescent cells at outlet (i), outlet (ii), and outlet (iii) was 58.6%, 22.4%, and 8.6%, respectively. Significantly more senescent cells were observed in the outlet (i) compared to the other two outlets. On the other hand, the senescent cell concentration from sham irradiated mouse bone marrow samples in the input, outlet (i), outlet (ii), and outlet (iii) were 4.2, 3.6, 3.2, and 3.8 per 1×10⁴ cells, respectively (FIG. 21F). There was no significant difference in senescent cell ratio among sample input and all three outlets. A higher senescence concentration from the bone marrow sample of 6.5 Gy irradiated mice compared to that from sham irradiated mice was observed. Because of the size increase in senescent cells in the 6.5 Gy irradiated mouse bone marrow samples, the senescent cell concentration in the outlet (i) was also significantly increased. The total cells (excluding red blood cells) in the 6.5 Gy irradiated mouse samples only 8.6% of that in the sham mice sample, indicating the severe reduction in cell numbers after X-ray radiation exposure.

As discussed above or elsewhere herein, the present disclosure provides a 2-stage device that incorporates 3D filter arrays with a tunable z-gap for the enrichment of the viable human MSCs by removal of senescent and necrotic cell populations. The 2-stage device could also isolate senescent mouse MSCs. The mouse MSC population after senescent cell removal grew faster on average than those before removal. Moreover, ˜58.6% of senescent cells found in bone marrow samples from mice treated with X-ray radiation could be collected at the outlet that is for senescent cell removal. By adjusting the z-gap, the device could separate different cell lines with different average cell sizes. The 3D filter array in the device may be optimized to prevent the clogging for live-cell separation and maintain the healthy states of cells for subsequent cell culture. This platform may provide the following advantages. It works directly with bone marrow samples with very minimum clogging and coagulation. The separation can operate in a continuous flow with a flow rate of 1.5 mL/hour. The 2-stage design can minimize the cell leaking to side channels and allow the usage of the peristaltic pump to make senescence/necrosis in vivo dialysis feasible.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-133. (canceled)

134. A microfluidic device, comprising:
a fluidic channel; and
an array of obstacles disposed in said fluidic channel comprising a first row of obstacles, wherein said first row of obstacles is oriented at an incline angle (θ) greater than 0° relative to a direction of a fluid flow in said fluidic channel,
wherein:
(i) a first obstacle of said first row of obstacles has a first parallelogram cross-section;
(ii) a second obstacle of said first row of obstacles is adjacent to said first obstacle, and has a second parallelogram cross-section; and
(iii) a first side of said first parallelogram substantially parallels a second side of said second parallelogram cross-section, said first side and said second side are facing each other.

135. The microfluidic device of claim 134, wherein said first obstacle has a height less than a height of said fluidic channel.

136. The microfluidic device of claim 135, wherein said first obstacle is immobilized on a top surface of said fluidic channel.

137. The microfluidic device of claim 135, wherein said first obstacle is immobilized on a bottom surface of said fluidic channel.

138. The microfluidic device of claim 134, wherein said first obstacle has a height equal to a height of said fluidic channel.

139. The microfluidic device of claim 134, wherein said incline angle (θ) is from 1° to 85°.

140. The microfluidic device of claim 139, wherein said incline angle (θ) is from 5° to 30°.

141. The microfluidic device of claim 134, wherein a distance between said array of obstacles and a side wall of said fluidic channel increases along said direction of said fluid flow.

142. The microfluidic device of claim 134, wherein an average spacing size between adjacent obstacles of said array is from 100 nanometers to 100 micrometers (μm).

143. The microfluidic device of claim 142, wherein said average spacing size is from 1 μm to 100 μm.

144. The microfluidic device of claim 134, wherein said array of obstacles further comprise a second row of obstacles.

145. The microfluidic device of claim 134, further comprising one or more fluid inlets in fluidic communication with said fluidic channel.

146. The microfluidic device of claim 134, further comprising a first fluid outlet and a second fluid outlet in fluidic communication with said fluidic channel.

147. The microfluidic device of claim 146, further comprising a capture array in fluidic communication with said second fluid outlet.

148. The microfluidic device of claim 146, further comprising a tubing in fluidic communication with said second fluid outlet.

149. A method comprising:
(a) directing a fluid comprising two or more target analytes into a microfluidic device, said microfluidic device comprising:
a fluidic channel; and
an array of obstacles disposed in said fluidic channel comprising a first row of obstacles,
wherein said first row of obstacles is oriented at an incline angle (θ) greater than 0° relative to a direction of a fluid flow in said fluidic channel,
wherein:
(i) a first obstacle of said first row of obstacles has a first parallelogram cross-section;
(ii) a second obstacle of said first row of obstacles is adjacent to said first obstacle, and has a second parallelogram cross-section; and
(iii) a first side of said first parallelogram substantially parallels a second side of said second parallelogram cross-section, said first side and said second side are facing each other
(b) directing said fluid to flow through said fluidic channel; and
(c) separating a target analyte from rest of said two or more target analytes using said array of obstacles upon flow of said fluid through said fluidic channel.

150. The method of claim 149, wherein said separating in (c) comprises: using said array of obstacles to direct at least a subset of said two or more target analytes to flow at a direction different from said direction of said fluid flow.

151. The method of claim 149, wherein said separating in (c) comprises: separating said target analyte from the rest of said two or more target analytes based at least partially on a size of said target analyte.

152. The method of claim 151, where said size of said target analyte is larger than an average spacing size between adjacent obstacles of said array.

153. The method of claim 152, wherein said average spacing size is from 100 nanometers to 100 micrometers (μm).
